(19) United States
(12) Patent Application Publication
(10) Pub. No.: US 2011/0306626 A1
(43) Pub. Date: Dec. 15, 2011

(54) IMIDAZOLINONE DERIVATIVES AS CGRP RECEPTOR ANTAGONISTS

(76) Inventors: Harold G. Selnick, Ambler, PA (US); Michael R. Wood, Brentwood, TN (US); Melody McWherter, Boyertown, PA (US); Ivory D. Hills, Harleysville, PA (US); Craig A. Stump, Pottstown, PA (US)

(21) Appl. No.: 13/139,610

(22) PCT Filed: Dec. 10, 2009
(86) PCT No.: PCT/US09/67412

§ 371 Date: Aug. 31, 2011

Related U.S. Application Data
(60) Provisional application No. 61/138,161, filed on Dec. 17, 2008.

(51) Int. Cl. A61K 31/437; C07D 491/107; C07D 403/12; C07D 471/20; A61K 31/4184; A61P 9/12; A61K 31/444; A61P 25/06; A61P 25/00; A61P 29/00; A61P 25/08; C07D 471/10; A61K 31/4439

(52) U.S. Cl. 514/278; 546/15; 548/301.1; 514/387

(57) ABSTRACT

The present invention is directed to imidazolinone derivatives which are antagonists of CGRP receptors and useful in the treatment or prevention of diseases in which CGRP is involved, such as migraine. The invention is also directed to pharmaceutical compositions comprising these compounds and the use of these compounds and compositions in the prevention or treatment of such diseases in which CGRP is involved.

### BACKGROUND OF THE INVENTION

CGRP (Calcitonin Gene-Related Peptide) is a naturally occurring 37-amino acid peptide that is generated by tissue-specific alternate processing of calcitonin messenger RNA and is widely distributed in the central and peripheral nervous system. CGRP is localized predominantly in sensory afferent and central neurons and mediates several biological actions, including vasodilation. CGRP is expressed in alpha- and beta-forms that vary by one and three amino acids in the rat and human, respectively. CGRP-alpha and CGRP-beta display similar biological properties. When released from the cell, CGRP initiates its biological responses by binding to specific cell surface receptors that are predominantly coupled to the activation of adenylyl cyclase. CGRP receptors have been identified and pharmacologically evaluated in several tissues and cells, including those of brain, cardiovascular, endothelial, and smooth muscle origin.

Based on pharmacological properties, these receptors are divided into at least two subtypes, denoted CGRP₁ and CGRP₂. Human α-CGRP-(8-37), a fragment of CGRP that lacks seven N-terminal amino acid residues, is a selective antagonist of CGRP₁, whereas the linear analogue of CGRP, diacetoamido methyl cysteine CGRP ([Cys(ACM)2,7]CGRP), is a selective agonist of CGRP₂. CGRP is a potent neuromodulator that has been implicated in the pathology of cerebrovascular disorders such as migraine and cluster headache. In clinical studies, elevated levels of CGRP in the jugular vein were found to occur during migraine attacks (Goadsby et al., Ann. Neurol., 1990, 28, 183-187), salivary levels of CGRP are elevated in migraine subjects between attacks (Bellamy et al., Headache, 2006, 46, 24-33), and CGRP itself has been shown to trigger migrainous headache (Lassen et al., Cephalalgia, 2002, 22, 54-61). In clinical trials, the CGRP antagonist BIBN4096BS has been shown to be effective in treating acute attacks of migraine (Olesen et al., New Engl. J. Med., 2004, 350, 1104-1110) and was able to prevent headache induced by CGRP infusion in a control group (Petersen et al., Clin. Pharmacol. Ther., 2005, 77, 202-213).

CGRP-mediated activation of the trigeminovascular system may play a key role in migraine pathogenesis. Additionally, CGRP activates receptors on the smooth muscle of intracranial vessels, leading to increased vasodilation, which is thought to contribute to headache pain during migraine attacks (Lance, Headache Pathogenesis: Monoamines, Neuropeptides, Purines and Nitric Oxide, Lippincott-Raven Publishers, 1997, 3-9). The middle meningeal artery, the principle artery in the dura mater, is innervated by sensory fibers from the trigeminal ganglion which contain several neuropeptides, including CGRP. Trigeminal ganglion stimulation in the cat resulted in increased levels of CGRP, and in humans, activation of the trigeminal system caused facial flushing and increased levels of CGRP in the external jugular vein (Goadsby et al., Ann. Neurol., 1988, 23, 193-196). Electrical stimulation of the dura mater in rats increased the diameter of the middle meningeal artery, an effect that was blocked by prior administration of CGRP(8-37), a peptide CGRP antagonist (Williamson et al., Cephalalgia, 1997, 17, 525-531). Trigeminal ganglion stimulation increased facial blood flow in the rat, which was inhibited by CGRP(8-37) (Escott et al., Brain Res. 1995, 669, 93-99). Electrical stimulation of the trigeminal ganglion in marmoset produced an increase in facial blood flow that could be blocked by the non-peptide CGRP antagonist BIBN4096BS (Doods et al., Br. J. Pharmacol., 2000, 129, 420-423). Thus the vascular effects of CGRP may be attenuated, prevented or reversed by a CGRP antagonist.

CGRP-mediated vasodilation of rat middle meningeal artery was shown to sensitize neurons of the trigeminal nucleus caudalis (Williamson et al., The CGRP Family: Calcitonin Gene-Related Peptide (CGRP), Amylin, and Adrenomedullin, Landes Bioscience, 2000, 245-247). Similarly, distention of dural blood vessels during migraine headache may sensitize trigeminal neurons. Some of the associated symptoms of migraine, including extra-cranial pain and facial allodynia, may be the result of sensitized trigeminal neurons (Burstein et al., Ann. Neural. 2000, 47, 614-624). A CGRP antagonist may be beneficial in attenuating, preventing or reversing the effects of neuronal sensitization.

The ability of the compounds of the present invention to act as CGRP antagonists makes them useful pharmacological agents for disorders that involve CGRP in humans and animals, but particularly in humans. Such disorders include migraine and cluster headache (Doods, Curr Opin Inves Drugs, 2001, 2 (9), 1261-1268; Edvinsson et al., Cephalalgia, 1994, 14, 320-327); chronic tension type headache (Ashina et al., Neurology, 2000, 14, 1335-1340); pain (Yu et al., Eur. J. Pharm., 1998, 347, 275-282); chronic pain (Hulsebosch et al., Pain, 2000, 86, 163-175); neurogenic inflammation and inflammatory pain (Holzer, Neurosci., 1988, 24, 739-768; Delay-Goyet et al., Acta Physiol. Scanda. 1992, 146, 537-538; Salmon et al., Nature Neurosci., 2001, 4(4), 357-358); eye pain (May et al. Cephalalgia, 2002, 22, 195-196), tooth pain (Awawdeh et al., Int. Endocrin. J., 2002, 35, 30-36), non-insulin dependent diabetes mellitus (Molina et al., Diabetes, 1990, 39, 260-265); vascular disorders; inflammation (Zhang et al., Pain, 2001, 89, 265), arthritis, bronchial hyperreactivity, asthma, (Foster et al., Ann. N Y Acad. Sci., 1992, 657, 397-404; Schini et al., Am. J. Physiol., 1994, 267, H2483-H2490; Zheng et al., J. Virol., 1993, 67, 5786-5791); shock, sepsis (Beer et al., Crit. Care Med., 2002, 30 (8), 1794-1798); opiate withdrawal syndrome (Salmon et al., Nature Neurosci., 2001, 4(4), 357-358); morphine tolerance (Menard et al., J. Neurosci., 1996, 16 (7), 2342-2351); hot flashes in men and women (Chen et al., Lancet, 1993, 342, 49; Spetz et al., J. Urology, 2001, 166, 1720-1723); allergic dermatitis (Wallengren, Contact Dermatitis, 2000, 43 (3), 137-143); psoriasis; encephalitis, brain trauma, ischaemia, stroke, epilepsy, and neurodegenerative diseases (Rohrenbeck et al., Neurobiol. of Disease 1999, 6, 15-34); skin diseases (Geppetti and Holzer, Eds., Neurogenic Inflammation, 1996, CRC Press, Boca Raton, Fla.), neurogenic cutaneous redness, skin rosaceousness and erythema; tinnitus (Herzog et al., J. Membrane Biology, 2002, 189(3), 225); inflammatory bowel disease, irritable bowel syndrome, (Hoffman et al. Scandinavian Journal of Gastroenterology, 2002, 37(4) 414-422) and cystitis. Of particular importance is the acute or prophylactic treatment of headache, including migraine and cluster headache.

The present invention relates to compounds that are useful as ligands for CGRP receptors, in particular antagonists for CGRP receptors, their use in therapy, pharmaceutical compositions comprising them and methods of therapy using them.

### SUMMARY OF THE INVENTION

The present invention is directed to imidazolinone derivatives which are antagonists of CGRP receptors and useful in the treatment or prevention of diseases in which CGRP is involved, such as migraine. The invention is also directed to pharmaceutical compositions comprising these compounds and the use of these compounds and compositions in the prevention or treatment of such diseases in which CGRP is involved.

### DETAILED DESCRIPTION OF THE INVENTION

The present invention encompasses a genus of compounds of the formula I:

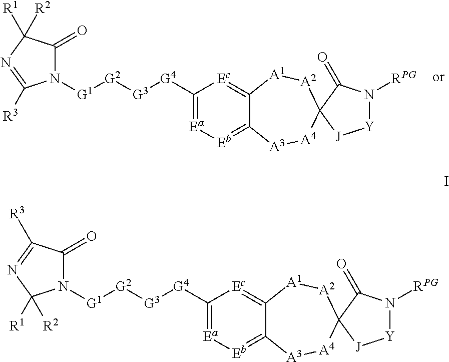

wherein:
A¹ and A³ are independently selected from:
(1) —O—,
(2) —S(O)ᵥ—,
(3) —Si(OR^{a})(C₁₋₄alkyl)-, where alkyl is unsubstituted or substituted with 1-5 halo,
(4) —Si(C₁₋₄alkyl)₂-, where each alkyl is independently unsubstituted or substituted with 1-5 halo,
(5) —CR^{e}R^{f}—,
(6) —N(R⁴)—,
(7) —(C═O)—, and
(8) a bond;
   A² and A4 are independently selected from:
(1) —O—,
(2) —S(O)ᵥ—,
(3) —Si(OR^{a})(C₁₋₄alkyl)-, where alkyl is unsubstituted or substituted with 1-5 halo,
(4) —Si(C₁₋₄alkyl)₂-, where each alkyl is independently unsubstituted or substituted with 1-5 halo,
(5) —CR^{e}R^{f}—,
(6) —N(R⁴)—, and
(7) —(C═O)—;
   E^{a} is selected from:
(1) —C(R^{5a})═,
(2) —N═, and
(3) —(N⁺—O⁻)═;
   E^{b} is selected from:
(1) —C(R^{5b})═,
(2) —N═, and
(3) —(N⁺—O⁻)═;
   E^{c} is selected from:
(1) —C(R^{5c})═,
(2) —N═, and
(3) —(N⁺—O⁻)═;
   G¹ is selected from:
(1) a bond,
(2) —CR^{e}R^{f}—,
(3) —CR^{e}R^{f}—CH₂—,
(4) —CH₂—CR^{e}R^{f}—, and
(5) —(C═O)—;
   G² is selected from:
(1) a bond,
(2) —CR^{e}R^{f}—,
(3) —CR^{e}R^{f}—CH₂—,
(4) —CH₂—CR^{e}R^{f}—,
(5) —(C═O)—,
(6) —N(R⁴)—,
(7) —O—,
(8) —S(O)ᵥ—,
(9) —SiR^{g}R^{h}—,
(10) —C(Rⁱ)═C(R^{j})—, and
(11) —C≡C—;
   G³ is selected from:
(1) a bond,
(2) —CR^{e}R^{f}—,
(3) —N(R⁴)—,
(4) —O—,
(5) —S(O)ᵥ—,
(6) —SiR^{g}R^{h}—,
(7) —C(Rⁱ)═C(R^{j})—,
(8) —C≡C—, and
(9) —(C═O)—;
   G⁴ is selected from:
(1) —CR^{e}R^{f}—,
(2) —N(R⁴)—,
(3) —O—,
(4) —S(O)ᵥ—,
(5) —SiR^{g}R^{h}—,
(6) —C(Rⁱ)═C(R^{j})—, and
(7) —C═C—;
   R¹ and R² are each independently selected from:
(1) hydrogen,
(2) —C₁₋₆alkyl, which is unsubstituted or substituted with 1-5 substituents each independently selected from:
   (a) halo,
   (b) —OR^{a},
   (c) —C₃₋₆cycloalkyl,
   (d) phenyl or heterocycle, wherein said heterocycle is selected from: pyridyl, pyrimidinyl, pyrazinyl, pyridazinyl, piperidinyl, piperazinyl, pyrrolidinyl, thienyl, morpholinyl, thiazolyl, indolyl, indazolyl, benzimidazolyl, and oxazolyl, which phenyl or heterocycle is unsubstituted or substituted with 1-5 substituents each independently selected from:
      (i) halo,
      (ii) —C₁₋₆alkyl, which is unsubstituted or substituted with 1-5 halo,
      (iii) —OR^{a},
      (iv) —NR^{b}R^{c},
      (v) —CN, and
      (vi) oxo,
   (e) —CO₂R^{a},
   (f) —C(═O)NR^{b}R^{c},
   (g) —S(O)ᵥR^{d},
   (h) —CN,
   (i) —NR^{b}R^{c},
   (j) —N(R^{b})C(═O)R^{a},
   (k) —N(R^{b})SO₂R^{d},
   (l) —CF₃,
   (m) —O—CO₂R^{d},
   (n) —O—(C═O)—NR^{b}R^{c},
   (O)—NR^{b}—(C═O)—NR^{b}R^{c}, and
   (p) —C(═O)R^{a},
(3) —C₃₋₈cycloalkyl, which is unsubstituted or substituted with 1-5 substituents each independently selected from:
   (a) halo,
   (b) —CN,
   (c) —C₁₋₄alkyl, which is unsubstituted or substituted with 1-3 halo, and
   (d) —OR^{a},
(4) phenyl or heterocycle, wherein said heterocycle is selected from: pyridyl, pyrimidinyl, pyrazinyl, pyridazinyl, piperidinyl, piperazinyl, pyrrolidinyl, thienyl, morpholinyl, thiazolyl and oxazolyl, which phenyl or heterocycle is unsubstituted or substituted with 1-5 substituents each independently selected from:
   (a) halo,
   (b) —OR^{a},
   (c) —C₃₋₆cycloalkyl,
   (d) phenyl, which is unsubstituted or substituted with 1-5 substituents each independently selected from:
      (i) halo,
      (ii) —C₁₋₆alkyl, which is unsubstituted or substituted with 1-6 halo, and
      (iii) —OR^{a},
   (e) —CO₂R^{a},
   (f) —C(═O)NR^{b}R^{c},
   (g) —S(O)ᵥR^{d},
   (h) —CN,
   (i) —NR^{b}R^{c},
   (j) —N(R^{b})C(═O)R^{a},
   (k) —N(R^{b})SO₂R^{d},
   (l) —O—CO₂R^{d},
   (m) —O—(C═O)—NR^{b}R^{c},
   (n) —NR^{b}—(C═O)—NR^{b}R^{c},
   (O)—C(═O)R^{a},
   (p) —C₁₋₆alkyl, which is unsubstituted or substituted with 1-6 halo, and
   (q) oxo,
(5) halo,
(6) —OR^{a},
(7) —CN,
(8) —CO₂R^{a},
(9) —N(R^{b})C(═O)R^{a},
(10) —NR^{b}R^{c},
(11) —C(═O)NR^{b}R^{c}, and
(12) ═O(C═O)R^{a};
or R¹ and R² and the carbon atom or atoms to which they are attached join to form a ring selected from cyclopropyl, cyclobutyl, cyclopentyl, cyclohexyl, cycloheptyl, cyclooctyl, cyclononyl, cyclobutenyl, cyclopentenyl, cyclohexenyl, cycloheptenyl, cyclooctenyl, dioxolanyl, dioxanyl, aziridinyl, azetidinyl, pyrrolidinyl, piperidinyl, piperazinyl, morpholinyl, tetrahydrofuranyl, tetrahydropyranyl, tetrahydrothiapyranyl, oxetanyl, thietanyl and tetrahydrothienyl, and benzofused analogs of any of the aforementioned rings, wherein any sulfur atoms present are optionally oxidized to the sulfone or sulfoxide, and said ring is unsubstituted or substituted with 1-6 substituents each independently selected from:
(a) —C₁₋₆alkyl, which is unsubstituted or substituted with 1-3 substituents each independently selected from:
   (i) halo,
   (ii) —OR^{a},
   (iii) —C₃₋₆cycloalkyl,
   (iv) —CO₂R^{a},
   (v) —NR^{b}R^{c},
   (vi) —S(O)ᵥR^{d},
   (vii) —C(═O)NR^{b}R^{c}, and
   (viii) phenyl,
(b) —C₃₋₆cycloalkyl, wherein the C₃₋₆cycloalkyl group is optionally fused to the ring, and which C₃₋₆cycloalkyl group is unsubstituted or substituted with 1-3 substituents each independently selected from:
   (i) halo,
   (ii) —OR^{a},
   (iii) —C₃₋₆cycloalkyl
   (iv) —CO₂R^{a},
   (v) —NR^{b}R^{c},
   (vi) —S(O)ᵥR^{d},
   (vii) —C(═O)NR^{b}R^{c}, and
   (viii) phenyl,
(c) phenyl or heterocycle, wherein heterocycle is selected from: pyridyl, pyrimidinyl, pyrazinyl, pyridazinyl, piperidinyl, piperazinyl, pyrrolidinyl, thienyl, morpholinyl, imidazolyl, furanyl, tetrahydrofuranyl, thiazolyl and oxazolyl, wherein the phenyl or heterocycle is optionally fused to the ring, and which phenyl or heterocycle is unsubstituted or substituted with 1-5 substituents each independently selected from:
   (i) halo,
   (ii) —C₁₋₆alkyl, which is unsubstituted or substituted with 1-5 halo,
   (iii) —OR^{a},
   (iv) —CO₂R^{a},
   (v) —O(C═O)R^{a},
   (vi) —CN,
   (vii) —NR^{b}R^{c},
   (viii) oxo,
   (ix) —C(═O)NR^{b}R^{c},
   (x) —N(R^{b})C(═O)R^{a},
   (xi) —N(R^{b})CO₂R^{a},
   (xii) —O(C═O)NR^{b}R^{c}, and
   (xiii) —S(O)ᵥR^{d},
(d) —OR^{a},
(e) —CO₂R^{a},
(O—C(═O)NR^{b}R^{c},
(g) —S(O)ᵥR^{d},
(h) —CN,
(i) halo,
(j) —NR^{b}R^{c},
(k) —N(R^{b})C(═O)R^{a},
(l) —N(R^{b})SO₂R^{d},
(m) —O—CO₂R^{d},
(n) —O—(C═O)—NR^{b}R^{c},
(O) —NR^{b}—(C═O)—NR^{b}R^{c},
(p) —C(═O)R^{a}, and
(q) oxo;
   R³ is selected from the group consisting of:
(1) —C₁₋₁₀alkyl, which is unsubstituted or substituted with 1-5 substituents each independently selected from:
   (a) halo,
   (b) —OR^{a},
   (c) —C₃₋₆cycloalkyl,
   (d) phenyl or heterocycle, wherein said heterocycle is selected from: pyridyl, pyrimidinyl, pyrazinyl, pyridazinyl, piperidinyl, piperazinyl, pyrrolidinyl, thienyl, morpholinyl, thiazolyl, indolyl, indazolyl, benzimidazolyl, and oxazolyl, which phenyl or heterocycle is unsubstituted or substituted with 1-5 substituents each independently selected from:
      (i) halo,
      (ii) —C₁₋₆alkyl, which is unsubstituted or substituted with 1-5 halo,
      (iii) —OR^{a},
      (iv) —NR^{b}R^{c},
      (v) —CN, and
      (vi) oxo,
   (e) —CO₂R^{a},
   (f) —C(═O)NR^{b}R^{c},
   (g) —S(O)ᵥR^{d},
   (h) —CN,
   (i) —NR^{b}R^{c},
   (j) —N(R^{b})C(═O)R^{a},
   (k) —N(R^{b})SO₂R^{d},
   (l) —CF₃,
   (m) —O—CO₂R^{d},
   (n) —O—(C═O)—NR^{b}R^{c},
   (p) —NR^{b}—(C═O)—NR^{b}R^{c}, and
   (q) —C(═O)R^{a},
(2) —C₃₋₁₀cycloalkyl or benzofused —C₃₋₁₀cycloalkyl, which is unsubstituted or substituted with 1-5 substituents each independently selected from:
   (a) halo,
   (b) —CN,
   (c) —C₁₋₄alkyl, which is unsubstituted or substituted with 1-3 halo, and
   (d) —OR^{a}, and
(3) phenyl, naphthyl, tetrahydronaphthyl, indanyl, biphenyl, phenanthryl, anthryl, azepinyl, azepanyl, azetidinyl, benzimidazolyl, benzisoxazolyl, benzopyranyl, benzofurazanyl, benzopyranyl, benzothiopyranyl, benzofuryl, 1,3-benzodioxolyl, benzothiazolyl, benzothienyl, benzoxazolyl, benzopyrazolyl, benzotriazolyl, chromanyl, cinnolinyl, dibenzofuranyl, dihydrobenzofuryl, dihydrobenzothienyl, dihydrobenzothiopyranyl, dihydrobenzothiopyranyl sulfone, furyl, furanyl, imidazolidinyl, imidazolinyl, imidazolyl, indolinyl, indolyl, isochromanyl, isoindolinyl, isoquinolinyl, isothiazolidinyl, isothiazolyl, morpholinyl, naphthyridinyl, oxazolyl, oxadiazolyl, 2-oxoazepinyl, 4-oxonaphthyridinyl, 2-oxopiperazinyl, 2-oxopiperidinyl, 2-oxopyrrolidinyl, 2-oxopyridyl, 2-oxoquinolinyl, piperidyl, piperazinyl, pyrazinyl, pyrazolidinyl, pyrazolyl, pyridazinyl, pyridinyl, pyridyl, pyrimidinyl, pyrimidyl, pyrrolidinyl, pyrrolyl, quinazolinyl, quinolinyl, quinoxalinyl, tetrahydrofuranyl, tetrahydrofuryl, tetrahydroimidazopyridinyl, tetrahydroisoquinolinyl, tetrahydroquinolinyl, tetrazolyl, thiamorpholinyl, thiamorpholinyl sulfoxide, thiamorpholinyl sulfone, thiazolyl, thiazolinyl, thienofuryl, thienothienyl, thienyl, triazolyl, isoxazolyl, tetrahydrothienyl, tetrahydropyranyl, oxetanyl, tetrahydrothiapyranyl, and thietanyl, each of which is unsubstituted or substituted with 1-5 substituents each independently selected from R⁶;
   R⁴ is independently selected from:
(1) hydrogen,
(2) —C(═O)R^{a},
(3) —CO₂R^{a},
(4) —S(═O)R^{d},
(5) —SO₂R^{d},
(6) —C(═O)NR^{b}R^{c},
(7) —C₁₋₆alkyl, which is unsubstituted or substituted with 1-5 substituents each independently selected from:
   (a) halo,
   (b) —OR^{a},
   (c) —C₃₋₆cycloalkyl,
   (d) phenyl or heterocycle, wherein said heterocycle is selected from: pyridyl, pyrimidinyl, pyrazinyl, pyridazinyl, piperidinyl, piperazinyl, pyrrolidinyl, thienyl, morpholinyl, thiazolyl and oxazolyl, which phenyl or heterocycle is unsubstituted or substituted with 1-5 substituents each independently selected from:
      (i) halo,
      (ii) which is unsubstituted or substituted with 1-5 halo,
      (iii) —OR^{a},
      (iv) —NR^{b}R^{c},
      (v) —C(═O)R^{a},
      (vi) —CO₂R^{a}, and
      (vii) oxo,
   (e) —CO₂R^{a},
   (f) —C(═O)NR^{b}R^{c},
   (g) —S(O)ᵥR^{d},
   (h) —CN,
   (i) —NR^{b}R^{c},
   (j) —N(R^{b})C(═O)R^{a},
   (k) —N(R^{b})SO₂R^{d},
   (l) —CF₃,
   (m) —O—CO₂R^{d},
   (n) —O—(C═O)—NR^{b}R^{c},
   (O)—NR^{b}—(C═O)—NR^{b}R^{c}, and
   (p) —C(═O)R^{a},
(8) —C₃₋₆cycloalkyl, which is unsubstituted or substituted with 1-6 substituents each independently selected from:
   (a) halo,
   (b) —CN,
   (c) —OR^{a}, and
   (d) C₁₋₆alkyl, which is unsubstituted or substituted with 1-6 halo; R^{5a}, R^{5b} and R^{5c} are each independently selected from:
(1) hydrogen,
(2) —C₁₋₆alkyl, which is unsubstituted or substituted with 1-6 halo,
(3) halo,
(4) —OR^{a}, and
(5) —CN;
   R⁶ is selected from:
(1) —C₁₋₆alkyl, which is unsubstituted or substituted with 1-5 substituents each independently selected from:
   (a) halo,
   (b) —OR^{a},
   (c) —C₃₋₆cycloalkyl,
   (d) phenyl or heterocycle, wherein said heterocycle is selected from: pyridyl, pyrimidinyl, pyrazinyl, pyridazinyl, piperidinyl, piperazinyl, pyrrolidinyl, thienyl, morpholinyl, thiazolyl and oxazolyl, which phenyl or heterocycle is unsubstituted or substituted with 1-5 substituents each independently selected from:
      (i) halo,
      (ii) —C₁₋₆alkyl, which is unsubstituted or substituted with 1-5 halo, and
      (iii) —OR^{a},
   (e) —CO₂R^{a},
   (f) —C(═O)NR^{b}R^{c},
   (g) —S(O)ᵥR^{d},
   (h) —CN,
   (i) —NR^{b}R^{c},
   (j) —N(R^{b})C(═O)R^{a},
   (k) —N(R^{b})SO₂R^{d},
   (l) —CF₃,
   (m) —O—CO₂R^{d},
   (n) —O—(C═O)—NR^{b}R^{c},
   (o) —NR^{b}—(C═O)—NR^{b}R^{c}, and
   (p) —C(═O)R^{a},
(2) —C₃₋₆cycloalkyl, which is unsubstituted or substituted with 1-5 substituents each independently selected from:
   (a) halo,
   (b) —CN,
   (c) —C₁₋₆alkyl, which is unsubstituted or substituted with 1-5 halo,
   (d) —OR^{a}, and
   (e) phenyl, which is unsubstituted or substituted with 1-5 substituents where the substituents are each independently selected from:
      (i) —OR^{a},
      (ii) halo,
      (iii) —CN, and
      (iv) —C₁₋₆alkyl, which is unsubstituted or substituted with 1-5 halo,
(3) phenyl or heterocycle, wherein said heterocycle is selected from: pyridyl, pyrimidinyl, pyrazinyl, pyridazinyl, piperidinyl, piperazinyl, pyrrolidinyl, thienyl, morpholinyl, thiazolyl and oxazolyl, which phenyl or heterocycle is unsubstituted or substituted with 1-5 substituents each independently selected from:
   (a) halo,
   (b) —OR^{a},
   (c) —C₃₋₆cycloalkyl,
   (d) phenyl, which is unsubstituted or substituted with 1-5 substituents each independently selected from:
      (i) halo,
      (ii) —C₁₋₆alkyl, which is unsubstituted or substituted with 1-6 halo, and
      (iii) —OR^{a},
   (e) —CO₂R^{a},
   (f) —C(═O)NR^{b}R^{c},
   (g) —S(O)ᵥR^{d},
   (h) —CN,
   (i) —NR^{b}R^{c},
   (j) —N(R^{b})C(═O)R^{a},
   (k)—N(R^{b})SO₂R^{d},
   (l) —O—CO₂R^{d},
   (m) —O—(C═O)—NR^{b}R^{c},
   (n) —NR^{b}—(C═O)—NR^{b}R^{c},
   (o)—C(═O)R^{a}, and
   (p) —C₁₋₆alkyl, which is unsubstituted or substituted with 1-6 halo,
(4) halo,
(5) oxo,
(6) —OR^{a},
(7) —CN,
(8) —CO₂R^{a},
(9) —C(═O)R^{a},
(10) —NR^{b}R^{c},
(11) —S(O)ᵥR^{d},
(12) —C(═O)NR^{b}R^{c},
(13) —O—CO₂R^{d},
(14) —N(R^{b})CO₂R^{d},
(15) —O—(C═O)—NR^{b}R^{c},
(16) —NR^{b}—(C═O)—NR^{b}R^{c},
(17) —SO₂NR^{b}R^{c},
(18) —N(R^{b})SO₂R^{d},
and two R⁶ groups on the same or adjacent atoms together with the atom(s) to which they are attached may join to form a ring selected from cyclopropyl, cyclobutyl, cyclopentyl, cyclohexyl, cycloheptyl, aziridinyl, azetidinyl, pyrrolidinyl, piperidinyl, piperazinyl, morpholinyl, thietanyl and tetrahydrothienyl, wherein the sulfur is optionally oxidized to the sulfone or sulfoxide, which ring is unsubstituted or substituted with 1-5 substituents each independently selected from:
   (a) —C₁₋₆alkyl, which is unsubstituted or substituted with 1-3 substituents each independently selected from:
      (i) halo,
      (ii) —OR^{a},
      (iii) —C₃₋₆cycloalkyl,
      (iv) —CO₂R^{a},
      (v) —NR^{b}R^{c},
      (vi) —S(O)ᵥR^{d},
      (vii) —C(═O)NR^{b}R^{c}, and
      (viii) phenyl,
   (b) phenyl or heterocycle, wherein said heterocycle is selected from: pyridyl, pyrimidinyl, pyrazinyl, pyridazinyl, piperidinyl, piperazinyl, pyrrolidinyl, thienyl, morpholinyl, thiazolyl and oxazolyl, which phenyl or heterocycle is unsubstituted or substituted with 1-5 substituents each independently selected from:
      (i) halo,
      (ii) —C₁₋₆alkyl, which is unsubstituted or substituted with 1-5 halo, and
      (iii) —OR^{a},
   (c) —OR^{a},
   (d) halo,
   (e) —CO₂R^{a},
   (f) —C(═O)NR^{b}R^{c},
   (g) —S(O)ᵥR^{d},
   (h) —CN,
   (i) —NR^{b}R^{c},
   (j) —N(R^{b})C(═O)R^{a},
   (k)—N(R^{b})SO₂R^{d},
   (l) —O—CO₂R^{d},
   (m)—O—(C═O)—NR^{b}R^{c},
   (n) —NR^{b}—(C═O)—NR^{b}R^{c}, and
   (o)—C(═O)R^{a};
      R^{PG} is selected from:
(1) hydrogen,
(2) —C₁₋₆alkyl which is unsubstituted or substituted with 1-5 halo,
(3) —CH₂OR^{a},
(4) —CH₂—O—CH₂CH₂Si(CH₃)₃,
(5) —CH₂OP(═O)(OR^{c})₂,
(6) —(CH₂)ₖ-phenyl, which is unsubstituted or substituted with 1-3 substituents each independently selected from:
   (a) halo,
   (b) —OR^{a},
   (c) —CN, and
   (d) —C₁₋₆alkyl, which is unsubstituted or substituted with 1-6 halo;
      J is selected from:
(1) ═C(R^{7a})—,
(2) —CR⁸R⁹—,
(3) —C(═O)—, and
(4) —N(R^{b})—;
   Y is selected from:
(1) ═C(R^{7b})—,
(2) —CR⁸R⁹—,
(3) —C(═O)—,
(4) and
(5) —N(R^{b})—;
   R^{7a} and R^{7b} are each independently selected from:
(1) hydrogen,
(2) —C₁₋₄alkyl, which is unsubstituted or substituted with 1-5 substituents each independently selected from:
   (a) halo,
   (b) —OR^{a},
   (c) —C₃₋₆cycloalkyl,
   (d) phenyl or heterocycle, wherein said heterocycle is selected from: imidazolyl, oxazolyl, pyridyl, pyrimidinyl, pyrazinyl, pyridazinyl, piperidinyl, piperazinyl, pyrrolidinyl, thiazolyl, thienyl, triazolyl, isoxazolyl and morpholinyl, which phenyl or heterocycle is unsubstituted or substituted with 1-3 substituents each independently selected from:
      (i) halo,
      (ii) —OR^{a},
      (iii) —CN, and
      (iv) C₁₋₆alkyl, which is unsubstituted or substituted with 1-6 halo,
(3) phenyl or heterocycle, wherein heterocycle is selected from: imidazolyl, oxazolyl, pyridyl, pyrimidinyl, pyrazinyl, pyridazinyl, tetrahydrofuryl, piperidinyl, piperazinyl, pyrrolidinyl, azetidinyl, thiazolyl, thienyl, triazolyl, isoxazolyl and morpholinyl, which phenyl or heterocycle is unsubstituted or substituted with 1-3 substituents each independently selected from:
   (a) halo,
   (b) —OR^{a},
   (c) —C₃₋₆cycloalkyl,
   (d) —C₁₋₄alkyl which is unsubstituted or substituted with 1-6 halo, and
   (e) phenyl, which is unsubstituted or substituted with 1-5 substituents each independently selected from:
      (i) halo,
      (ii) —C₁₋₆alkyl, which is unsubstituted or substituted with 1-6 halo, and
      (iii) —OR^{a},
(4) halo,
(5) —OR^{a},
(6) —CN,
(7) —CO₂R^{a},
(8) —NR^{b}R^{c}, and
(9) —C(═O)NR^{b}R^{c};
or R^{7a} and R^{7b} and the atom(s) to which they are attached join to form a ring selected from cyclopentenyl, cyclohexenyl, phenyl, pyridyl, pyrimidinyl, pyrazinyl, pyridazinyl, furanyl, dihydropyranyl, dihydropyranyl, thiazolyl, isothiazolyl, oxazolyl, isoxazolyl, imidazolyl, triazolyl, thienyl, dihydrothienyl and dihydrothiopyranyl, which ring is unsubstituted or substituted with 1-5 substituents each independently selected from:
   (a) —C₁₋₆alkyl, which is unsubstituted or substituted with 1-3 substituents each independently selected from:
      (i) halo,
      (ii) —OR^{a},
      (iii) —C₃₋₆cycloalkyl,
      (iv) phenyl or heterocycle, wherein heterocycle is selected from pyridyl, pyrimidinyl, pyrazinyl, pyridazinyl, piperidinyl, piperazinyl, pyrrolidinyl, thienyl and morpholinyl, which phenyl or heterocycle is unsubstituted or substituted with 1-5 substituents each independently selected from:
         (I) —OR^{a},
         (II) halo,
         (III) —CN, and
         (IV) —C₁₋₆alkyl which is unsubstituted or substituted with 1-6 halo,
      (v) —CO₂R^{a},
      (vi) —NR^{b}R^{c},
      (vii) —S(O)ᵥR^{d},
      (viii) —C(═O)NR^{b}R^{c},
      (ix) —N(R^{b})CO₂R^{a}, and
      (x) —N(R^{b})SO₂R^{d},
   (b) phenyl or heterocycle, wherein said heterocycle is selected from pyridyl, pyrimidinyl, pyrazinyl, pyridazinyl, piperidinyl, azetidinyl, piperazinyl, pyrrolidinyl, thienyl and morpholinyl, which phenyl or heterocycle is unsubstituted or substituted with 1-5 substituents each independently selected from:
      (i) halo,
      (ii) —OR^{a},
      (iii) —CN, and
      (iv) —C₁₋₆alkyl which is unsubstituted or substituted with 1-6 halo,
   (c) halo,
   (d) —S(O)ᵥR^{d},
   (e) —OR^{a},
   (f) —CN,
   (g) —C(═O)R^{a},
   (h) —NR^{b}R^{c},
   (i) —C(═O)NR^{b}R^{c},
   (j) —CO₂R^{a},
   (k) —(NR^{b})CO₂R^{a},
   (l) —O—(C═O)—NR^{b}R^{c},
   (m) —(NR^{b})—(C═O)—NR^{b}R^{c},
   (n) oxido,
   (O) oxo, and
   (p) —(NR^{b})SO₂R^{d};
      R⁸ and R⁹ are each independently selected from:
(1) hydrogen,
(2) halo,
(3) —OR^{a},
(4) —C₁₋₆alkyl, which is unsubstituted or substituted with 1-4 substituents each independently selected from:
   (a) halo,
   (b) —OR^{a},
   (c) —CN,
   (d) phenyl or heterocycle, wherein said heterocycle is selected from pyridyl, pyrimidinyl, thienyl, pyridazinyl, piperidinyl, azetidinyl, piperazinyl, pyrrolidinyl, morpholinyl, tetrahydrofuranyl, tetrahydropyranyl and pyrazinyl, which phenyl or heterocycle is unsubstituted or substituted with 1-5 substituents each independently selected from:
      (i) —OR^{a},
      (ii) halo,
      (iii) —CN,
      (iv) —C₁₋₆alkyl which is unsubstituted or substituted with 1-6 halo,
(5) phenyl or heterocycle wherein heterocycle is selected from pyridyl, pyrimidinyl, thienyl, pyridazinyl, piperidinyl, azetidinyl, piperazinyl, pyrrolidinyl, morpholinyl, tetrahydrofuranyl, tetrahydropyranyl and pyrazinyl, which phenyl or heterocycle is unsubstituted or substituted with 1-5 substituents each independently selected from:
(a) halo,
(b) —CN,
(c)—OR^{a},
(d) nitro,
(e) —C₁₋₆alkyl which is unsubstituted or substituted with 1-6 halo; and R⁸ and R⁹ and the atom to which they are attached may join to form a 4-, 5-, or 6-membered ring optionally containing a heteroatom selected from N, O, and S, wherein the sulfur is optionally oxidized to the sulfone or sulfoxide, which ring is unsubstituted or substituted with 1-4 substituents each independently selected from:
(a) halo,
(b) —OR^{a},
(c) —C₁₋₆alkyl, which is unsubstituted or substituted with 1-6 halo, and
(d) phenyl;
   R^{a} is independently selected from:
(1) hydrogen,
(2) C₁₋₆alkyl, which is unsubstituted or substituted with 1-7 substituents each independently selected from:
   (a) halo,
   (b) —O—C₁₋₆alkyl, which is unsubstituted or substituted with 1-6 halo,
   (c) hydroxyl,
   (d) —CN, and
   (e) phenyl or heterocycle wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:
      (i) halo,
      (ii) —O—C₁₋₆alkyl, which is unsubstituted or substituted with 1-6 halo,
      (iii) —CN,
      (iv) nitro,
      (v) hydroxyl, and
      (vi) —C₁₋₆alkyl, which is unsubstituted or substituted with 1-6 halo,
(3) phenyl or heterocycle wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:
   (a) halo,
   (b) —CN,
   (c) —O—C₁₋₆alkyl, which is unsubstituted or substituted with 1-6 halo,
   (d) nitro,
   (e) hydroxyl, and
   (f) —C₁₋₆alkyl, which is unsubstituted or substituted with 1-6 halo, and
(4) —C₃₋₆cycloalkyl, which is unsubstituted or substituted with 1-6 halo;
   R^{b} and R^{c} are independently selected from:
(1) hydrogen,
(2) C₁₋₆alkyl, which is unsubstituted or substituted with 1-7 substituents each independently selected from:
   (a) halo,
   (b) —OR^{a},
   (c) —CN,
   (d) —CO₂R^{a},
   (e) phenyl or heterocycle, wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:
      (i) halo,
      (ii) —OR^{a},
      (iii) —C₁₋₆alkyl, which is unsubstituted or substituted with 1-6 halo, and
      (iv) nitro,
(3) phenyl or heterocycle, wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:
   (a) halo,
   (b) —OR^{a},
   (c) —C₁₋₆alkyl, which is unsubstituted or substituted with 1-6 halo,
   (d) —C₃₋₆cycloalkyl, which is unsubstituted or substituted with 1-6 halo,
   (e) —CN, and
   (f) —CO₂R^{a},
(4) —C₃₋₆cycloalkyl, which is unsubstituted or substituted with 1-6 halo; and R^{b} and R^{c} and the nitrogen to which they are attached may join to form a 4-, 5-, or 6-membered ring optionally containing an additional heteroatom selected from N, O, and S, wherein the sulfur is optionally oxidized to the sulfone or sulfoxide, which ring is unsubstituted or substituted with 1-4 substituents each independently selected from:
(a) halo,
(b) —OR^{a}, and
(c) —C₁₋₆alkyl, which is unsubstituted or substituted with 1-6 halo, and
(d) phenyl;
   R^{d} is independently selected from:
(1) C₁₋₆alkyl, which is unsubstituted or substituted with 1-4 substituents each independently selected from:
   (a) halo,
   (b) —OR^{a},
   (c)—CO₂R^{a},
   (d) —CN, and
   (e) phenyl or heterocycle, wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:
      (i) halo,
      (ii) —OR^{a},
      (iii) —C₁₋₆alkyl, which is unsubstituted or substituted with 1-6 halo, and
      (iv) nitro,
(2) phenyl or heterocycle, wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:
   (a) halo,
   (b) —OR^{a},
   (c) —C₁₋₆alkyl, which is unsubstituted or substituted with 1-6 halo,
   (d) —C₃₋₆cycloalkyl, which is unsubstituted or substituted with 1-6 halo
   (e) —CN, and
   (f) —CO₂R^{a}, and
(3) —C₃₋₆cycloalkyl, which is unsubstituted or substituted with 1-6 halo;
   R^{e} and R^{f} are independently selected from:
(1) hydrogen,
(2) —C₁₋₄alkyl, which is unsubstituted or substituted with 1-6 halo,
(3) —OR^{a},
(4) —CN,
(5) halo,
(6) phenyl, and
(7) benzyl;
and R^{e} and R^{f} and the carbon atom or atoms to which they are attached may join to form a 3-, 4-, 5-, or 6-membered ring optionally containing a heteroatom selected from N, O, and S, wherein the sulfur is optionally oxidized to the sulfone or sulfoxide, which ring is unsubstituted or substituted with 1-4 substituents each independently selected from:
   (a) halo,
   (b) —OR^{a},
   (c) —C₁₋₆alkyl, which is unsubstituted or substituted with 1-6 halo, and
   (d) phenyl;
      R^{g} and R^{h} are independently selected from:
(1) —C₁₋₄alkyl, which is unsubstituted or substituted with 1-6 halo,
(2) —OR^{a},
(3) —C₃₋₆cycloalkyl, which is unsubstituted or substituted with 1-6 halo,
(4) phenyl, and
(5) benzyl;
and R^{g} and R^{h} and the silicon atom to which they are attached may join to form a 3-, 4-, 5-, or 6-membered ring optionally containing a heteroatom selected from N, O, and S, wherein the sulfur is optionally oxidized to the sulfone or sulfoxide, which ring is unsubstituted or substituted with 1-4 substituents each independently selected from:
   (a) halo,
   (b) —OR^{a},
   (c) —C₁₋₄alkyl, which is unsubstituted or substituted with 1-3 halo, and
   (d) phenyl;
      Rⁱ and R^{j} are independently selected from:
(1) hydrogen,
(2) —C₁₋₄alkyl, which is unsubstituted or substituted with 1-6 halo,
(3) —OR^{a},
(4) halo,
(5) phenyl, and
(6) benzyl;
   v is 0, 1, or 2;
   k is 0, 1, or 2;
   and pharmaceutically acceptable salts thereof and individual enantiomers and diastereomers thereof.

Within the genus, the invention encompasses a first sub-genus of compounds of formula Ia:

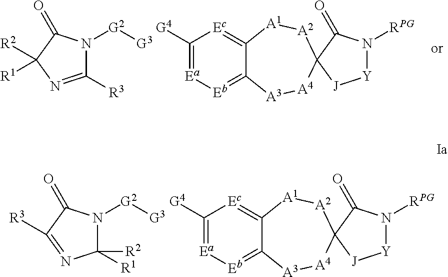

and pharmaceutically acceptable salts thereof.

Also within the genus, the invention encompasses a second sub-genus of compounds of formula Ib:

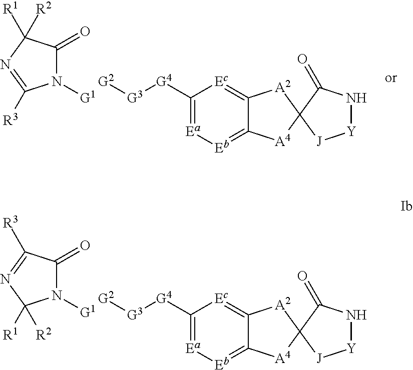

and pharmaceutically acceptable salts thereof.

Also within the genus, the invention encompasses a third sub-genus of compounds of formula Ic:

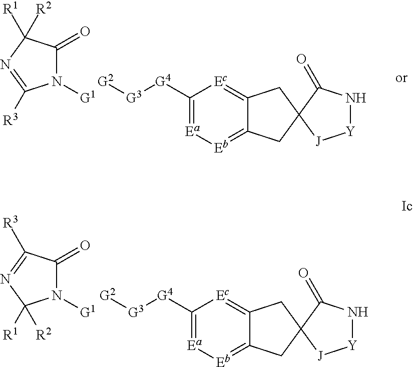

and pharmaceutically acceptable salts thereof.

Also within the genus, the invention encompasses a fourth sub-genus of compounds of formula Id:

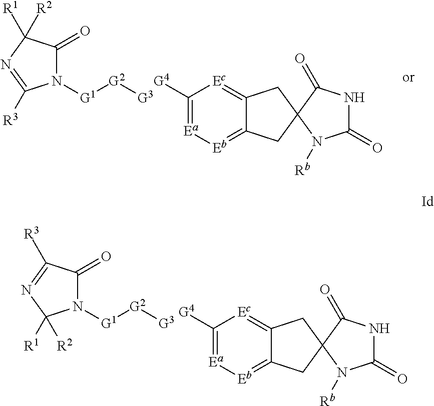

and pharmaceutically acceptable salts thereof.

Also within the genus, the invention encompasses a fifth sub-genus of compounds of formula Ie:

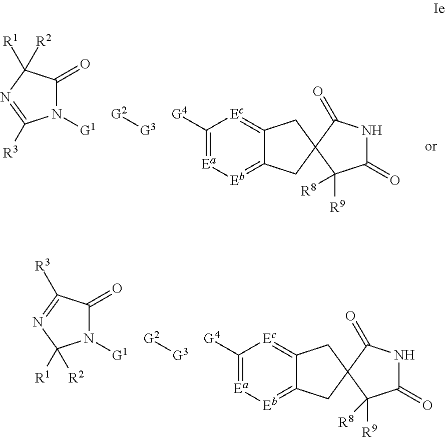

and pharmaceutically acceptable salts thereof.

Also within the genus, the invention encompasses a sixth sub-genus of compounds of formula If:

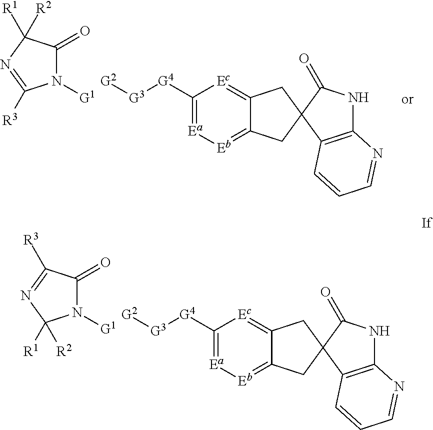

and pharmaceutically acceptable salts thereof.

Also within the genus, the invention encompasses a seventh sub-genus of compounds of formula I in which R^{5a}, R^{5b} and R^{5c} are independently selected from hydrogen, halo, and —C₁₋₆alkyl, which is unsubstituted or substituted with 1-5 fluoro.

Also within the genus, the invention encompasses an eighth sub-genus of compounds of formula I in which R¹ and R² are independently selected from:
(1) hydrogen,
(2) —C₁₋₆alkyl, which is unsubstituted or substituted with 1-5 substituents where the substitutents are each independently selected from: halo, phenyl, and —OR^{a},
(3) —C₃₋₆cycloalkyl, which is unsubstituted or substituted with 1-5 fluoro,
(4) phenyl or heterocycle, which is unsubstituted or substituted with 1-5 halo,
(5) halo,
(6) —OR^{a},
(7) —NR^{b}R^{c}, and
(8) —O(C═O)R^{a}.

Also within the genus, the invention encompasses a ninth sub-genus of compounds of formula I in which R¹ and R² and the carbon atom or atoms to which they are attached join to form a ring selected from cyclopropyl, cyclobutyl, cyclopentyl, cyclohexyl, cycloheptyl, cyclooctyl, cyclopentenyl, cyclohexenyl, cycloheptenyl, dioxolanyl, dioxanyl, tetrahydrofuranyl, tetrahydropyranyl, pyrrolidinyl, and piperidinyl, which ring is unsubstituted or substituted with 1-6 substituents each independently selected from:
(1) —C₁₋₆alkyl, which is unsubstituted or substituted with 1-3 substituents where the substitutents are each independently selected from: halo, —OR^{a}, and phenyl,
(2) —C₃₋₆cycloalkyl, wherein the C₃₋₆cycloalkyl group is optionally fused to the ring, and which C₃₋₆cycloalkyl group is unsubstituted or substituted with 1-3 substituents each independently selected from: halo, —OR^{a}, and phenyl,
(3) phenyl or heterocycle, wherein heterocycle is selected from: pyridyl, pyrimidinyl, pyrazinyl, pyridazinyl, piperidinyl, piperazinyl, pyrrolidinyl, thienyl, thiazolyl and oxazolyl, wherein the phenyl or heterocycle is optionally fused to the ring, and which phenyl or heterocycle is unsubstituted or substituted with 1-3 substituents each independently selected from: halo, —OR^{a}, and —C₁₋₄alkyl, which is unsubstituted or substituted with 1-5 fluoro,
(4) halo,
(5) oxo,
(6) —CO₂R^{a}, and
(7) —C(═O)R^{a}.

Also within the genus, the invention encompasses a tenth sub-genus of compounds of formula I in which R⁴ is selected from: hydrogen, —C(═O)R^{a}, —CO₂Rₐ, —SO₂R_{d}, and which is unsubstituted or substituted with 1-5 fluoro.

Also within the genus, the invention encompasses an eleventh sub-genus of compounds of formula I in which J is ═C(R^{7a})—, —CR⁸R⁹— or —N(R^{b})—.

Also within the genus, the invention encompasses a twelfth sub-genus of compounds of formula I in which Y is ═C(R^{7b})—, —CR⁸R⁹— or —C(═O)—.

Also within the genus, the invention encompasses a thirteenth sub-genus of compounds of formula I in which R^{7a} and R^{7b} are independently selected from:
(1) hydrogen,
(2) —C₁₋₄alkyl, which is unsubstituted or substituted with 1-3 substituents each independently selected from: halo, —OR^{a}, —C₃₋₆cycloalkyl, and phenyl,
(3) phenyl or heterocycle, wherein heterocycle is selected from: pyridyl, pyrimidinyl, pyrazinyl, thiazolyl, thienyl, triazolyl, isoxazolyl and morpholinyl, which phenyl or heterocycle is unsubstituted or substituted with 1-3 substituents each independently selected from: —C₁₋₄alkyl which is unsubstituted or substituted with 1-3 halo, —OR^{a}, and halo,
(4) halo,
(5) OR^{a}, and
(6) —NR^{b}R^{c}.

Also within the genus, the invention encompasses a fourteenth sub-genus of compounds of formula I in which R^{7a} and R^{7b} and the atom(s) to which they are attached join to form a ring selected from cyclohexenyl, phenyl, pyridyl, pyrimidinyl, pyrazinyl, pyridazinyl, furanyl, oxazolyl, isoxazolyl, imidazolyl, and thienyl, which ring is unsubstituted or substituted with 1-3 substituents each independently selected from:
(1) —C₁₋₆alkyl, which is unsubstituted or substituted with 1-3 substituents each independently selected from: halo, OR^{a}, —CO₂R^{a}, —NR^{b}R^{c}, and CONR^{b}R^{c},
(2) phenyl or heterocycle, wherein heterocycle is selected from pyridyl, pyrimidinyl, pyrazinyl, pyridazinyl, piperidinyl, azetidinyl, piperazinyl, pyrrolidinyl, thienyl and morpholinyl, which phenyl or heterocycle is unsubstituted or substituted with 1-3 substituents each independently selected from: halo, OR^{a} and —C₁₋₄alkyl, which is unsubstituted or substituted with 1-3 fluoro,
(3) halo,
(4) OR^{a},
(5) —CN,
(6) —NR^{b}R^{c},
(7) CONR^{b}R^{c}, and
(8) oxo.

Also within the genus, the invention encompasses a fifteenth sub-genus of compounds of formula Ig:

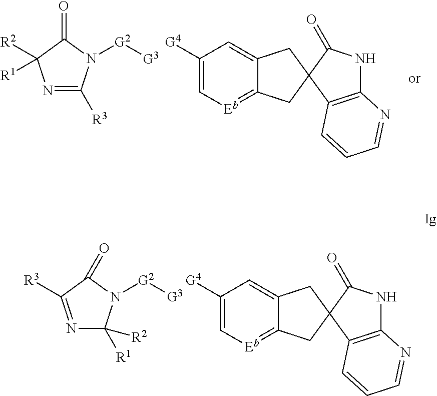

wherein:
R¹ and R² are independently selected from:
(1) —C₁₋₆alkyl, which is unsubstituted or substituted with 1-5 substituents each independently selected from:
   (a) halo,
   (b) —OR^{a},
   (c) —C₃₋₆cycloalkyl,
   (d) phenyl or heterocycle, wherein said heterocycle is selected from: pyridyl, pyrimidinyl, pyrazinyl, pyridazinyl, piperidinyl, piperazinyl, pyrrolidinyl, thienyl, morpholinyl, thiazolyl, indolyl, indazolyl, benzimidazolyl, and oxazolyl, which phenyl or heterocycle is unsubstituted or substituted with 1-5 substituents each independently selected from:
      (i) halo,
      (ii) —C₁₋₆alkyl, which is unsubstituted or substituted with 1-5 halo,
      (iii) —OR^{a},
      (iv) NR^{b}R^{c},
      (v) —CN, and
      (vi) oxo;
   (e) —CO₂R^{a},
   (f) —C(═O)NR^{b}R^{c},
   (g) —S(O)ᵥR^{d},
   (h) —CN,
   (i) —NR^{b}R^{c},
   (j) —N(R^{b})C(═O)R^{a},
   (k) —N(R^{b})SO₂R^{d},
   (l) —CF₃,
   (m)—O—CO₂R^{d},
   (n) —O—(C═O)—NR^{b}R^{c},
   (O)—NR^{b}—(C═O)—NR^{b}R^{c}, and
   (p) —C(═O)R^{a}, and
(2) phenyl or heterocycle, wherein said heterocycle is selected from: pyridyl, pyrimidinyl, pyrazinyl, pyridazinyl, piperidinyl, piperazinyl, pyrrolidinyl, thienyl, morpholinyl, thiazolyl and oxazolyl, which phenyl or heterocycle is unsubstituted or substituted with 1-5 halo;
   or R¹ and R² and the carbon atom or atoms to which they are attached join to form a ring selected from cyclopropyl, cyclobutyl, cyclopentyl, cyclohexyl, cycloheptyl, cyclooctyl, cyclononyl, cyclobutenyl, cyclopentenyl, cyclohexenyl, cycloheptenyl, cyclooctenyl, dioxolanyl, dioxanyl, aziridinyl, azetidinyl, pyrrolidinyl, piperidinyl, piperazinyl, morpholinyl, tetrahydrofuranyl, tetrahydropyranyl, tetrahydrothiapyranyl, oxetanyl, thietanyl and tetrahydrothienyl, wherein the sulfur is optionally oxidized to the sulfone or sulfoxide, which ring is unsubstituted or substituted with 1-6 substituents each independently selected from:
(a) —C₁₋₆alkyl, which is unsubstituted or substituted with 1-3 substituents each independently selected from:
   (i) halo,
   (ii) —OR^{a},
   (iii) —C₃₋₆cycloalkyl,
   (iv) —CO₂R^{a},
   (v) —NR^{b}R^{c},
   (vi) —S(O)ᵥR^{d},
   (vii) —C(—-0)NR^{b}R^{c}, and
   (viii) phenyl,
(b) —C₃₋₆cycloalkyl, wherein the C₃₋₆cycloalkyl group is optionally fused to the ring, and which C₃₋₆cycloalkyl group is unsubstituted or substituted with 1-3 substituents each independently selected from:
   (i) halo,
   (ii) —OR^{a},
   (iii) —C₃₋₆cycloalkyl,
   (iv) —CO₂R^{a},
   (v) —NR^{b}R^{c},
   (vi) —S(O)ᵥR^{d},
   (vii) —C(═O)NR^{b}R^{c}, and
   (viii) phenyl,
(c) phenyl or heterocycle, wherein heterocycle is selected from: pyridyl, pyrimidinyl, pyrazinyl, pyridazinyl, piperidinyl, piperazinyl, pyrrolidinyl, thienyl, morpholinyl, imidazolyl, furanyl, tetrahydrofuranyl, thiazolyl and oxazolyl, wherein the phenyl or heterocycle is optionally fused to the ring, and which phenyl or heterocycle is unsubstituted or substituted with 1-5 substituents each independently selected from:
   (i) halo,
   (ii) —C₁₋₆alkyl, which is unsubstituted or substituted with 1-5 halo,
   (iii) —OR^{a},
   (iv) —CO₂R^{a},
   (v) —O(C═O)R^{a},
   (vi) —CN,
   (vii) —NR^{b}R^{c},
   viii) oxo,
   (ix) —C(═O)NR^{b}R^{c},
   (x) —N(R^{b})C(═O)R^{a},
   (xi) —N(R^{b})CO₂R^{a},
   (xii) —O(C═O)NR^{b}R^{c}, and
   (xiii) —S(O)ᵥR^{d},
(d) —OR^{a},
(e) —CO₂R^{a},
(f) —C(═O)NR^{b}R^{c},
(g) —S(O)ᵥR^{d},
(h) —CN,
(i) halo,
(j) —NR^{b}R^{c},
(k) —N(R^{b})C(═O)R^{a},
(l) —N(R^{b})SO₂R^{d},
(m)—O—CO₂R^{d},
(n) —O—(C═O)—NR^{b}R^{c},
(O)—NR^{b}—(C═O)—NR^{b}R^{c},
(q) —C(═O)R^{a}, and
(r) oxo;
   R³ is selected from:
(1) —C₃₋₁₀cycloalkyl or benzofused —C₃₋₁₀cycloalkyl, which is unsubstituted or substituted with 1-5 halo, and
(2) phenyl, naphthyl, tetrahydronaphthyl, indanyl, biphenyl, phenanthryl, anthryl, azepinyl, azepanyl, azetidinyl, benzimidazolyl, benzisoxazolyl, benzofuranyl, benzofurazanyl, benzopyranyl, benzothiopyranyl, benzofuryl, 1,3-benzodioxolyl, benzothiazolyl, benzothienyl, benzoxazolyl, benzopyrazolyl, benzotriazolyl, chromanyl, cinnolinyl, dibenzofuranyl, dihydrobenzofuryl, dihydrobenzothienyl, dihydrobenzothiopyranyl, dihydrobenzothiopyranyl sulfone, furyl, furanyl, imidazolidinyl, imidazolinyl, imidazolyl, indolinyl, indolyl, isochromanyl, isoindolinyl, isoquinolinyl, isothiazolidinyl, isothiazolyl, morpholinyl, naphthyridinyl, oxazolyl, oxadiazolyl, 2-oxoazepinyl, 4-oxonaphthyridinyl, 2-oxopiperazinyl, 2-oxopiperidinyl, 2-oxopyrrolidinyl, 2-oxopyridyl, 2-oxoquinolinyl, piperidyl, piperazinyl, pyrazinyl, pyrazolidinyl, pyrazolyl, pyridazinyl, pyridinyl, pyridyl, pyrimidinyl, pyrimidyl, pyrrolidinyl, pyrrolyl, quinazolinyl, quinolinyl, quinoxalinyl, tetrahydrofuranyl, tetrahydrofuryl, tetrahydroimidazopyridinyl, tetrahydroisoquinolinyl, tetrahydroquinolinyl, tetrazolyl, thiamorpholinyl, thiamorpholinyl sulfoxide, thiamorpholinyl sulfone, thiazolyl, thiazolinyl, thienofuryl, thienothienyl, thienyl, triazolyl, isoxazolyl, tetrahydrothienyl, tetrahydropyranyl, oxetanyl, tetrahydrothiapyranyl, and thietanyl, each of which is unsubstituted or substituted with 1-5 substituents each independently selected from R⁶;
   -G²-G³-G⁴- is selected from the group consisting of:

### —CH₂—C(O)—NH—

### —CH₂—CH₂—CH₂—,

### —CH₂—CH═CH—,

### —CH₂—CH≡C— and

### —CH₂—CH₃—O—;

and pharmaceutically acceptable salts thereof.

Within the fifteenth sub-genus, the invention encompasses a first class of compounds in which G²-G³-G⁴ is —CH₂—C(O)—NH— and pharmaceutically acceptable salts thereof.

Also within the genus, the invention encompasses a sixteenth sub-genus of compounds of formula Ih:

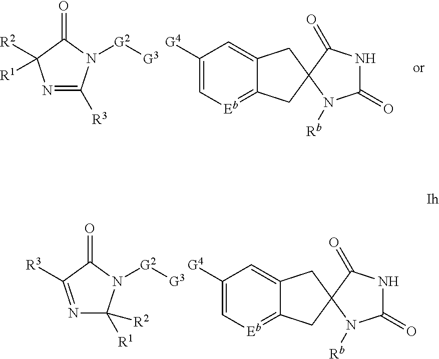

wherein:
R¹ and R² are independently selected from:
(1) —C₁₋₆alkyl, which is unsubstituted or substituted with 1-5 substituents each independently selected from:
   (a) halo,
   (b) —OR^{a},
   (c) —C₃₋₆cycloalkyl,
   (d) phenyl or heterocycle, wherein said heterocycle is selected from: pyridyl, pyrimidinyl, pyrazinyl, pyridazinyl, piperidinyl, piperazinyl, pyrrolidinyl, thienyl, morpholinyl, thiazolyl, indolyl, indazolyl, benzimidazolyl, and oxazolyl, which phenyl or heterocycle is unsubstituted or substituted with 1-5 substituents each independently selected from:
      (i) halo,
      (ii) —C₁₋₆alkyl, which is unsubstituted or substituted with 1-5 halo,
      (iii) —OR^{a},
      (iv) —NR^{b}R^{c},
      (v) —CN, and
      (vi) oxo;
   (e) —CO₂R^{a},
   (f) —C(═O)NR^{b}R^{c},
   —S(O)ᵥR^{d},
   (h) —CN,
   (i) —NR^{b}R^{c},
   (j) —N(R^{b})C(═O)R^{a},
   (k)—N(R^{b})SO₂R^{d},
   (l) —CF₃,
   (m) —O—CO₂R^{d},
   (n) —O—(C═O)—NR^{b}R^{c},
   (O)—NR^{b}—(C═O)—NR^{b}R^{c}, and
   (p) —C(═O)R^{a}, and
(2) phenyl or heterocycle, wherein said heterocycle is selected from: pyridyl, pyrimidinyl, pyrazinyl, pyridazinyl, piperidinyl, piperazinyl, pyrrolidinyl, thienyl, morpholinyl, thiazolyl and oxazolyl, which phenyl or heterocycle is unsubstituted or substituted with 1-5 halo;
   or R¹ and R² and the carbon atom or atoms to which they are attached join to form a ring selected from cyclopropyl, cyclobutyl, cyclopentyl, cyclohexyl, cycloheptyl, cyclooctyl, cyclononyl, cyclobutenyl, cyclopentenyl, cyclohexenyl, cycloheptenyl, cyclooctenyl, dioxolanyl, dioxanyl, aziridinyl, azetidinyl, pyrrolidinyl, piperidinyl, piperazinyl, morpholinyl, tetrahydrofuranyl, tetrahydropyranyl, tetrahydrothiapyranyl, oxetanyl, thietanyl and tetrahydrothienyl, wherein the sulfur is optionally oxidized to the sulfone or sulfoxide, which ring is unsubstituted or substituted with 1-6 substituents each independently selected from:
(a) —C₁₋₆alkyl, which is unsubstituted or substituted with 1-3 substituents each independently selected from:
   (i) halo,
   (ii) —OR^{a},
   (iii) —C₃₋₆cycloalkyl,
   (iv) —CO₂R^{a},
   (v) —NR^{b}R^{c},
   (vi) —S(O)ᵥR^{d},
   (vii) —C(═O)NR^{b}R^{c}, and
   (viii) phenyl,
(b) —C₃₋₆cycloalkyl, wherein the C₃₋₆cycloalkyl group is optionally fused to the ring, and which C₃₋₆cycloalkyl group is unsubstituted or substituted with 1-3 substituents each independently selected from:
   (i) halo,
   (ii) —OR^{a},
   (iii) —C₃₋₆cycloalkyl,
   (iv) —CO₂R^{a},
   (v) —NR^{b}R^{c},
   (vi) —S(O)ᵥR^{d},
   (vii) —C(═O)NR^{b}R^{c}, and
   (viii) phenyl,
(c) phenyl or heterocycle, wherein heterocycle is selected from: pyridyl, pyrimidinyl, pyrazinyl, pyridazinyl, piperidinyl, piperazinyl, pyrrolidinyl, thienyl, morpholinyl, imidazolyl, furanyl, tetrahydrofuranyl, thiazolyl and oxazolyl, wherein the phenyl or heterocycle is optionally fused to the ring, and which phenyl or heterocycle is unsubstituted or substituted with 1-5 substituents each independently selected from:
   (i) halo,
   (ii) —C₁₋₆alkyl, which is unsubstituted or substituted with 1-5 halo,
   (iii) —OR^{a},
   (iv) —CO₂R^{a},
   (v) —O(C═O)R^{a},
   (vi) —CN,
   (vii) —NR^{b}R^{c},
   (viii) oxo,
   (ix) —C(═O)NR^{b}R^{c},
   (x) —N(R^{b})C(═O)R^{a},
   (xi) —N(R^{b})CO₂R^{a},
   (xii) —O(C═O)NR^{b}R^{c}, and
   (xiii) —S(O)ᵥR^{d},
(d) —OR^{a},
(e) —CO₂R^{a},
(f) —C(═O)NR^{b}R^{c},
(g) —S(O)ᵥR^{d},
(h) —CN,
(i) halo,
(j) —NR^{b}R^{c},
(k) —N(R^{b})C(═O)R^{a},
(l) —N(R^{b})SO₂R^{d},
(m)—O—CO₂R^{d},
(n) —O—(C═O)—NR^{b}R^{c},
(O)—NR^{b}—(C═O)—NR^{b}R^{c},
(q) —C(═O)R^{a}, and
(r) oxo;
   R³ is selected from:
(1) —C₃₋₁₀cycloalkyl or benzofused —C₃₋₁₀cycloalkyl, which is unsubstituted or substituted with 1-5 halo, and
(2) phenyl, naphthyl, tetrahydronaphthyl, indanyl, biphenyl, phenanthryl, anthryl, azepinyl, azepanyl, azetidinyl, benzimidazolyl, benzisoxazolyl, benzofuranyl, benzofurazanyl, benzopyranyl, benzothiopyranyl, benzofuryl, 1,3-benzodioxolyl, benzothiazolyl, benzothienyl, benzoxazolyl, benzopyrazolyl, benzotriazolyl, chromanyl, cinnolinyl, dibenzofuranyl, dihydrobenzofuryl, dihydrobenzothienyl, dihydrobenzothiopyranyl, dihydrobenzothiopyranyl sulfone, furyl, furanyl, imidazolidinyl, imidazolinyl, imidazolyl, indolinyl, indolyl, isochromanyl, isoindolinyl, isoquinolinyl, isothiazolidinyl, isothiazolyl, morpholinyl, naphthyridinyl, oxazolyl, oxadiazolyl, 2-oxoazepinyl, 4-oxonaphthyridinyl, 2-oxopiperazinyl, 2-oxopiperidinyl, 2-oxopyrrolidinyl, 2-oxopyridyl, 2-oxoquinolinyl, piperidyl, piperazinyl, pyrazinyl, pyrazolidinyl, pyrazolyl, pyridazinyl, pyridinyl, pyridyl, pyrimidinyl, pyrimidyl, pyrrolidinyl, pyrrolyl, quinazolinyl, quinolinyl, quinoxalinyl, tetrahydrofuranyl, tetrahydrofuryl, tetrahydroimidazopyridinyl, tetrahydroisoquinolinyl, tetrahydroquinolinyl, tetrazolyl, thiamorpholinyl, thiamorpholinyl sulfoxide, thiamorpholinyl sulfone, thiazolyl, thiazolinyl, thienofuryl, thienothienyl, thienyl, triazolyl, isoxazolyl, tetrahydrothienyl, tetrahydropyranyl, oxetanyl, tetrahydrothiapyranyl, and thietanyl, each of which is unsubstituted or substituted with 1-5 substituents each independently selected from R⁶;
   -G²-G³-G⁴- is selected from the group consisting of:

### —CH₂—C(O)—NH—,

### —CH₂—CH₂—CH₂—,

### —CH₂—CH═CH—,

### —CH₂—C≡C— and

### —CH₂—CH₃—O—;

and pharmaceutically acceptable salts thereof.

Within the sixteenth sub-genus, the invention encompasses a first class of compounds in which G2-G3-G4 is —CH₂—C(O)—NH— and pharmaceutically acceptable salts thereof.

The invention also encompasses any of the examples that follow and pharmaceutically acceptable salts thereof.

The invention also encompasses a pharmaceutical composition which comprises an inert carrier and the compound of formula I.

The invention also encompasses a method for antagonism of CGRP receptor activity in a mammal which comprises the administration of an effective amount of the compound of formula I.

The invention also encompasses a method for treating, controlling, ameliorating or reducing the risk of headache, migraine or cluster headache in a mammalian patient in need of such which comprises administering to the patient a therapeutically effective amount of the compound of formula I.

The invention also encompasses a method of treating or preventing migraine headaches, cluster headaches, and headaches, said method comprising the co-administration, to a person in need of such treatment, of a therapeutically effective amount of the compound of claim 1 or a pharmaceutically acceptable salt thereof; and a therapeutically effective amount of a second agent selected from serotonin agonists, analgesics, anti-inflamatory agents, anti-hypertensives and anticonvulsants.

It is to be understood that where one or more of the above recited structures or substructures recite multiple substituents having the same designation each such variable may be the same or different from each similarly designated variable. For example, if R⁶ is recited multiple times in an embodiment of formula I, each instance of R⁶ in formula I may independently be any of the substructures defined under R⁶. The invention is not limited to structures and substructures wherein each R⁶ must be the same for a given structure. The same is true with respect to any variable appearing multiple times in a structure or substructure.

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. Any formulas, structures or names of compounds described in this specification that do not specify a particular stereochemistry are meant to encompass any and all existing isomers as described above and mixtures thereof in any proportion. When stereochemistry is specified, the invention is meant to encompass that particular isomer in pure form or as part of a mixture with other isomers in any proportion.

Some of the compounds described herein contain olefinic double bonds, and unless specified otherwise, are meant to include both E and Z geometric isomers.

In the compounds of generic formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

Tautomers of compounds defined in formula I are also included within the scope of the present invention. For example, compounds including carbonyl —CH₂C(═O)— groups (keto forms) may undergo tautomerism to form hydroxyl —CH═C(OH)— groups (enol forms). Both keto and enol forms are included within the scope of the present invention.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

As will be appreciated by those of skill in the art, not all of the R¹ and R² substituents are capable of forming a ring structure. Moreover, even those substituents capable of ring formation may or may not form a ring structure.

Also as appreciated by those of skill in the art, halo or halogen as used herein are intended to include chloro, fluoro, bromo and iodo.

As used herein, “alkyl” is intended to mean linear, branched and cyclic structures having no carbon-to-carbon double or triple bonds. Thus C₁₋₆alkyl is defined to identify the group as having 1, 2, 3, 4, 5 or 6 carbons in a linear or branched arrangement, such that C₁₋₆alkyl specifically includes, but is not limited to, methyl, ethyl, n-propyl, iso-propyl, n-butyl, iso-butyl, tert-butyl, pentyl and hexyl. “Cycloalkyl” is an alkyl, part or all of which forms a ring of three or more atoms. “Cycloalkyl” include mono-, bi- or tri-cyclic structures, including bridged structures such as adamantanyl. C₀ or C₀alkyl is defined to identify the presence of a direct covalent bond.

The term “alkenyl” means linear or branched structures and combinations thereof, of the indicated number of carbon atoms, having at least one carbon-to-carbon double bond, wherein hydrogen may be replaced by an additional carbon-to-carbon double bond. C₂₋₆alkenyl, for example, includes ethenyl, propenyl, 1-methylethenyl, butenyl and the like.

The term “alkynyl” means linear or branched structures and combinations thereof, of the indicated number of carbon atoms, having at least one carbon-to-carbon triple bond. Thus C₂₋₆alkynyl is defined to identify the group as having 2, 3, 4, 5 or 6 carbons in a linear or branched arrangement, such that C₂₋₆alkynyl specifically includes 2-hexynyl and 2-pentynyl.

As used herein, “aryl” is intended to mean any stable monocyclic or bicyclic carbon ring of up to 7 members in each ring, wherein at least one ring is aromatic. Examples of such aryl elements include phenyl, napthyl, tetrahydronapthyl, indanyl, or biphenyl.

The term “heterocycle” or “heterocyclic”, as used herein except where noted, represents a stable 4- to 8-membered monocyclic or stable 8- to 12-membered bicyclic heterocyclic ring system which is either saturated or unsaturated, and which consists of carbon atoms and from one to six heteroatoms selected from the group consisting of N, O, S, P and Si, and wherein the nitrogen, sulfur and phosphorus heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure. Examples of such heterocyclic groups include, but are not limited to, azetidine, chroman, dihydrofuran, dihydropyran, dioxane, dioxolane, hexahydroazepine, imidazolidine, imidazolidinone, imidazoline, imidazolinone, indoline, isochroman, isoindoline, isothiazoline, isothiazolidine, isoxazoline, isoxazolidine, morpholine, morpholinone, oxazoline, oxazolidine, oxazolidinone, oxetane, 2-oxohexahydroazepin, 2-oxopiperazine, 2-oxopiperidine, 2-oxopyrrolidine, piperazine, piperidine, pyran, pyrazolidine, pyrazoline, pyrrolidine, pyrroline, quinuclidine, tetrahydrofuran, tetrahydropyran, thiamorpholine, thiazoline, thiazolidine, thiomorpholine and N-oxides thereof.

The term “heteroaryl”, as used herein except where noted, represents a stable 5- to 7-membered monocyclic- or stable 9- to 10-membered fused bicyclic heterocyclic ring system which contains an aromatic ring, any ring of which may be saturated, such as piperidinyl, partially saturated, or unsaturated, such as pyridinyl, and which consists of carbon atoms and from one to six heteroatoms selected from the group consisting of N, O, S, P and Si, and wherein the nitrogen, sulfur and phosphorus heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure. Examples of such heteroaryl groups include, but are not limited to, benzimidazole, benzisothiazole, benzisoxazole, benzofuran, benzothiazole, benzothiophene, benzotriazole, benzoxazole, carboline, cinnoline, furan, furazan, imidazole, indazole, indole, indolizine, isoquinoline, isothiazole, isoxazole, naphthyridine, oxadiazole, oxazole, phthalazine, pteridine, purine, pyran, pyrazine, pyrazole, pyridazine, pyridine, pyrimidine, pyrrole, quinazoline, quinoline, quinoxaline, tetrazole, thiadiazole, thiazole, thiophene, triazine, triazole, and N-oxides thereof.

The term “alkoxy,” as in C₁-C₆ alkoxy, is intended to refer to include alkoxy groups of from 1 to 6 carbon atoms of a straight, branched and cyclic configuration. Examples include methoxy, ethoxy, propoxy, isopropoxy, cyclopropyloxy, cyclohexyloxy and the like.

The variables set forth in the generic descriptions that appear multiple times are independently selected from the indicated groups. For example, A¹ and A⁴ both include —S(O)ᵥ— in their definitions and v is defined as 0, 1 or 2. Thus, A¹ can be —S(O)₁— and A⁴ can be —S(O)₂—. The variable v is not required to be the same in both instances.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. In one aspect of the invention the salts are citric, hydrobromic, hydrochloric, maleic, phosphoric, sulfuric, fumaric, and tartaric acids. It will be understood that, as used herein, references to the compounds of Formula I are meant to also include the pharmaceutically acceptable salts.

Exemplifying the invention is the use of the compounds disclosed in the Examples and herein. Specific compounds within the present invention include a compound which is selected from the group consisting of the compounds disclosed in the following Examples and pharmaceutically acceptable salts thereof and individual diastereomers thereof.

The subject compounds are useful in a method of antagonism of CGRP receptors in a patient such as a mammal in need of such antagonism comprising the administration of an effective amount of the compound. The present invention is directed to the use of the compounds disclosed herein as antagonists of CGRP receptors. In addition to primates, especially humans, a variety of other mammals can be treated according to the method of the present invention.

Another embodiment of the present invention is directed to a method for the treatment, control, amelioration, or reduction of risk of a disease or disorder in which the CGRP receptor is involved in a patient that comprises administering to the patient a therapeutically effective amount of a compound that is an antagonist of CGRP receptors.

The present invention is further directed to a method for the manufacture of a medicament for antagonism of CGRP receptors activity in humans and animals comprising combining a compound of the present invention with a pharmaceutical carrier or diluent.

The subject treated in the present methods is generally a mammal, for example a human being, male or female, in whom antagonism of CGRP receptor activity is desired. The term. “therapeutically effective amount” means the amount of the subject compound that will elicit the biological or medical response of a tissue, system, animal or human that is being sought by the researcher, veterinarian, medical doctor or other clinician. As used herein, the term “treatment” refers both to the treatment and to the prevention or prophylactic therapy of the mentioned conditions, particularly in a patient who is predisposed to such disease or disorder.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The present invention includes within its scope prodrugs of the compounds of this invention. In general, such prodrugs will be functional derivatives of the compounds of this invention which are readily convertible in vivo into the required compound. Thus, in the methods of treatment of the present invention, the terms “administration of” or “administering a” compound shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this invention into the biological milieu.

The utility of the compounds in accordance with the present invention as antagonists of CGRP receptor activity may be demonstrated by methodology known in the art. Inhibition of the binding of ¹²⁵I-CGRP to receptors and functional antagonism of CGRP receptors were determined as follows:

NATIVE RECEPTOR BINDING ASSAY: The binding of ¹²⁵I-CGRP to receptors in SK—N-MC cell membranes was carried out essentially as described (Edvinsson et al. (2001) Eur. J. Pharmacol. 415, 39-44). Briefly, membranes (25 μg) were incubated in 1 mL of binding buffer [10 mM HEPES, pH 7.4, 5 mM MgCl₂ and 0.2% bovine serum albumin (BSA)] containing 10 μM ¹²⁵I-CGRP and antagonist. After incubation at room temperature for 3 h, the assay was terminated by filtration through GFB glass fibre filter plates (PerkinElmer) that had been blocked with 0.5% polyethyleneimine for 3 h. The filters were washed three times with ice-cold assay buffer (10 mM HEPES, pH 7.4 and 5 mM MgCl₂), then the plates were air dried. Scintillation fluid (50 μL) was added and the radioactivity was counted on a Topcount (Packard Instrument). Data analysis was carried out by using Prism and the Kᵢ was determined by using the Cheng-Prusoff equation (Cheng & Prusoff (1973) Biochem. Pharmacol. 22, 3099-3108).

RECOMBINANT RECEPTOR: Human CL receptor (Genbank accession number L76380) was subcloned into the expression vector pIREShyg2 (BD Biosciences Clontech) as a 5′NheI and 3′ PmeI fragment. Human RAMP1 (Genbank accession number A1001014) was subcloned into the expression vector pIRESpuro2 (BD Biosciences Clontech) as a 5′NheI and 3′NotI fragment. HEK 293 cells (human embryonic kidney cells; ATCC # CRL-1573) were cultured in DMEM with 4.5 g/L glucose, 1 mM sodium pyruvate and 2 mM glutamine supplemented with 10% fetal bovine serum (FBS), 100 units/mL penicillin and 100 μg/mL streptomycin, and maintained at 37° C. and 95% humidity. Cells were subcultured by treatment with 0.25% trypsin with 0.1% EDTA in HBSS. Stable cell line generation was accomplished by co-transfecting 10 μg of DNA with 30 μg Lipofectamine 2000 (Invitrogen) in 75 cm² flasks. CL receptor and RAMP1 expression constructs were co-transfected in equal amounts. Twenty-four hours after transfection the cells were diluted and selective medium (growth medium+300 μg/mL hygromycin and 1 μg/mL puromycin) was added the following day. A clonal cell line was generated by single cell deposition utilizing a FACS Vantage SE (Becton Dickinson). Growth medium was adjusted to 150 μg/mL hygromycin and 0.5 μg/mL puromycin for cell propagation.

RECOMBINANT RECEPTOR BINDING ASSAY: Cells expressing recombinant human CL receptor/RAMP1 were washed with PBS and harvested in harvest buffer containing 50 mM HEPES, 1 mM EDTA and Complete protease inhibitors (Roche). The cell suspension was disrupted with a laboratory homogenizer and centrifuged at 48,000 g to isolate membranes. The pellets were resuspended in harvest buffer plus 250 mM sucrose and stored at −70° C. For binding assays, 20 μg of membranes were incubated in 1 ml binding buffer (10 mM HEPES, pH 7.4, 5 mM MgCl₂, and 0.2% BSA) for 3 hours at room temperature containing 10 pM ¹²⁵I-hCGRP (GE Healthcare) and antagonist. The assay was terminated by filtration through 96-well GFB glass fiber filter plates (PerkinElmer) that had been blocked with 0.05% polyethyleneimine. The filters were washed 3 times with ice-cold assay buffer (10 mM HEPES, pH 7.4 and 5 mM MgCl₂). Scintillation fluid was added and the plates were counted on a Topcount (Packard). Non-specific binding was determined and the data analysis was carried out with the apparent dissociation constant (Kᵢ) determined by using a non-linear least squares fitting the bound CPM data to the equation below:

Y_{obsd}=(Yₘₐₓ−Yₘᵢₙ)(%Iₘₐₓ−%Iₘᵢₙ/100)+Yₘᵢₙ+(Yₘₐₓ−Yₘᵢₙ)(100−%Iₘₐₓ/100)

1+([Drug]/Kᵢ(1+[Radiolabel]/K_{d})nH

Where Y is observed CPM bound, Yₘₐₓ is total bound counts, Yₘᵢₙ is non specific bound counts, (Yₘₐₓ Yₘᵢₙ) is specific bound counts, % Iₘₐₓ is the maximum percent inhibition, % 1 min is the minimum percent inhibition, radiolabel is the probe, and the K_{d} is the apparent dissociation constant for the radioligand for the receptor as determined by Hot saturation experiments.

RECOMBINANT RECEPTOR FUNCTIONAL ASSAY: Cells were plated in complete growth medium at 85,000 cells/well in 96-well poly-D-lysine coated plates (Corning) and cultured for ˜19 h before assay. Cells were washed with PBS and then incubated with inhibitor for 30 min at 37° C. and 95% humidity in Cellgro Complete Serum-Free/Low-Protein medium (Mediatech, Inc.) with L-glutamine and 1 g/L BSA. Isobutyl-methylxanthine was added to the cells at a concentration of 300 μM and incubated for 30 min at 37° C. Human α-CGRP was added to the cells at a concentration of 0.3 nM and allowed to incubate at 37° C. for 5 min. After α-CGRP stimulation the cells were washed with PBS and processed for cAMP determination utilizing the two-stage assay procedure according to the manufacturer's recommended protocol (CAMP SPA direct screening assay system; RPA 559; GE Healthcare). Dose response curves were plotted and IC₅₀ values determined from a 4-parameter logistic Et as defined by the equation y=((a−d)/(1+(x/c)^{b})+d, where y=response, x=dose, a=max response, d=min response, c=inflection point and b=slope.

In particular, Examples 1 to 111 had activity as antagonists of the CGRP receptor in the aforementioned assays, generally with a Kᵢ or IC₅₀ value of less than about 1 μM. Such a result is indicative of the intrinsic activity of the compounds in use as antagonists of CGRP receptors.

The ability of the compounds of the present invention to act as CGRP antagonists makes them useful pharmacological agents for disorders that involve CGRP in humans and animals, but particularly in humans.

The compounds of the present invention have utility in treating, preventing, ameliorating, controlling or reducing the risk of one or more of the following conditions or diseases: headache; migraine; cluster headache; chronic tension type headache; pain; chronic pain; neurogenic inflammation and inflammatory pain; neuropathic pain; eye pain; tooth pain; diabetes; non-insulin dependent diabetes mellitus; vascular disorders; inflammation; arthritis; bronchial hyperreactivity, asthma; shock; sepsis; opiate withdrawal syndrome; morphine tolerance; hot flashes in men and women; allergic dermatitis; psoriasis; encephalitis; brain trauma; epilepsy; neurodegenerative diseases; skin diseases; neurogenic cutaneous redness, skin rosaceousness and erythema; inflammatory bowel disease, irritable bowel syndrome, cystitis; and other conditions that may be treated or prevented by antagonism of CGRP receptors. Of particular importance is the acute or prophylactic treatment of headache, including migraine and cluster headache.

The subject compounds are further useful in a method for the prevention, treatment, control, amelioration, or reduction of risk of the diseases, disorders and conditions noted herein.

The subject compounds are further useful in a method for the prevention, treatment, control, amelioration, or reduction of risk of the aforementioned diseases, disorders and conditions in combination with other agents.

The compounds of the present invention may be used in combination with one or more other drugs in the treatment, prevention, control, amelioration, or reduction of risk of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy may also include therapies in which the compound of Formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

For example, the present compounds may be used in conjunction with an anti-migraine agent, such as ergotamine and dihydroergotamine, or other serotonin agonists, especially a 5-HT_{1B/1D} agonist, for example sumatriptan, naratriptan, zolmitriptan, eletriptan, almotriptan, frovatriptan, donitriptan, and rizatriptan, a 5-HT_{1D} agonist such as PNU-142633 and a 5-HT_{1F} agonist such as LY334370; a cyclooxygenase inhibitor, such as a selective cyclooxygenase-2 inhibitor, for example rofecoxib, etoricoxib, celecoxib, valdecoxib or paracoxib; a non-steroidal anti-inflammatory agent or a cytokine-suppressing anti-inflammatory agent, for example with a compound such as ibuprofen, ketoprofen, fenoprofen, naproxen, indomethacin, sulindac, meloxicam, piroxicam, tenoxicam, lornoxicam, ketorolac, etodolac, mefenamic acid, meclofenamic acid, flufenamic acid, tolfenamic acid, diclofenac, oxaprozin, apazone, nimesulide, nabumetone, tenidap, etanercept, tolmetin, phenylbutazone, oxyphenbutazone, diflunisal, salsalate, olsalazine or sulfasalazine and the like; or glucocorticoids. Similarly, the instant compounds may be administered with an analgesic such as aspirin, acetaminophen, phenacetin, fentanyl, sufentanil, methadone, acetyl methadyl, buprenorphine or morphine.

Additionally, the present compounds may be used in conjunction with an interleukin inhibitor, such as an interleukin-1 inhibitor; an NK-1 receptor antagonist, for example aprepitant and fosaprepitant; an NMDA antagonist; an NR2B antagonist; a bradykinin-1 receptor antagonist; an adenosine A1 receptor agonist; a sodium channel blocker, for example lamotrigine; an opiate agonist such as levomethadyl acetate or methadyl acetate; a lipoxygenase inhibitor, such as an inhibitor of 5-lipoxygenase; an alpha receptor antagonist, for example indoramin; an alpha receptor agonist; a vanilloid receptor antagonist; a renin inhibitor; a granzyme B inhibitor; an endothelin antagonist; a norepinephrin precursor; anti-anxiety agents such as diazepam, alprazolam, chlordiazepoxide and chlorazepate; serotonin 5HT2 receptor antagonists; opiod agonists such as codeine, hydrocodone, tramadol, dextropropoxyphene and febtanyl; an mGluR5 agonist, antagonist or potentiator; a GABA A receptor modulator, for example acamprosate calcium; nicotinic antagonists or agonists including nicotine; muscarinic agonists or antagonists; a selective serotonin reuptake inhibitor, for example fluoxetine, paroxetine, sertraline, duloxetine, escitalopram, or citalopram; an antidepressant, for example amitriptyline, nortriptyline, clomipramine, imipramine, venlafaxine, doxepin, protriptyline, desipramine, trimipramine, or imipramine; a leukotriene antagonist, for example montelukast or zafirlukast; an inhibitor of nitric oxide or an inhibitor of the synthesis of nitric oxide.

Also, the present compounds may be used in conjunction with gap junction inhibitors; neuronal calcium channel blockers such as civamide; AMPA/KA antagonists such as LY293558; sigma receptor agonists; and vitamin 132.

Also, the present compounds may be used in conjunction with ergot alkaloids other than ergotamine and dihydroergotamine, for example ergonovine, ergonovine, methylergonovine, metergoline, ergoloid mesylates, dihydroergocornine, dihydroergocristine, dihydroergocryptine, dihydro-α-ergocryptine, dihydro-β-ergocryptine, ergotoxine, ergocornine, ergocristine, ergocryptine, α-ergocryptine, 3-ergocryptine, ergosine, ergostane, bromocriptine, or methysergide.

Additionally, the present compounds may be used in conjunction with a beta-adrenergic antagonist such as timolol, propanolol, atenolol, metoprolol or nadolol, and the like; a MAO inhibitor, for example phenelzine; a calcium channel blocker, for example flunarizine, diltiazem, amlodipine, felodipine, nisolipine, isradipine, nimodipine, lomerizine, verapamil, nifedipine, or prochlorperazine; neuroleptics such as olanzapine, droperidol, prochlorperazine, chlorpromazine and quetiapine; an anticonvulsant such as topiramate, zonisamide, tonabersat, carabersat, levetiracetam, lamotrigine, tiagabine, gabapentin, pregabalin or divalproex sodium; an anti-hypertensive such as an angiotensin II antagonist, for example losartan, irbesartin, valsartan, eprosartan, telmisartan, olmesartan, medoxomil, candesartan and candesartan cilexetil, an angiotensin 1 antagonist, an angiotensin converting enzyme inhibitor such as lisinopril, enalapril, captopril, benazepril, quinapril, perindopril, ramipril and trandolapril; or botulinum toxin type A or B.

The present compounds may be used in conjunction with a potentiator such as caffeine, an H2-antagonist, simethicone, aluminum or magnesium hydroxide; a decongestant such as oxymetazoline, epinephrine, naphazoline, xylometazoline, propylhexedrine, or levo-desoxy-ephedrine; an antitussive such as caramiphen, carbetapentane, or dextromethorphan; a diuretic; a prokinetic agent such as metoclopramide or domperidone; a sedating or non-sedating antihistamine such as acrivastine, azatadine, bromodiphenhydramine, brompheniramine, carbinoxamine, chlorpheniramine, clemastine, dexbrompheniramine, dexchlorpheniramine, diphenhydramine, doxylamine, loratadine, phenindamine, pheniramine, phenyltoloxamine, promethazine, pyrilamine, terfenadine, triprolidine, phenylephrine, phenylpropanolamine, or pseudoephedrine. The present compounds also may be used in conjunction with anti-emetics.

In a particular embodiment the present compounds are used in conjunction with an anti-migraine agent, such as: ergotamine or dihydroergotamine; a 5-HT₁ agonist, especially a 5-HT_{1B/1D} agonist, in particular, sumatriptan, naratriptan, zolmitriptan, eletriptan, almotriptan, frovatriptan, donitriptan, avitriptan and rizatriptan, and other serotonin agonists; and a cyclooxygenase inhibitor, such as a selective cyclooxygenase-2 inhibitor, in particular, rofecoxib, etoricoxib, celecoxib, valdecoxib or paracoxib.

The above combinations include combinations of a compound of the present invention not only with one other active compound, but also with two or more other active compounds. Likewise, compounds of the present invention may be used in combination with other drugs that are used in the prevention, treatment, control, amelioration, or reduction of risk of the diseases or conditions for which compounds of the present invention are useful. Such other drugs may be administered, by a route and in an amount commonly used therefore, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the compound of the present invention to the other active ingredient(s) may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, or from about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s), and via the same or different routes of administration.

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, solutions, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release. Oral tablets may also be formulated for immediate release, such as fast melt tablets or wafers, rapid dissolve tablets or fast dissolve films.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the faun of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane dial. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions and the like, containing the compounds of the present invention are employed. Similarly, transdermal patches may also be used for topical administration.

The pharmaceutical composition and method of the present invention may further comprise other therapeutically active compounds as noted herein which are usually applied in the treatment of the above mentioned pathological conditions.

In the treatment, prevention, control, amelioration, or reduction of risk of conditions which require antagonism of CGRP receptor activity an appropriate dosage level will generally be about 0.01 to 500 mg per kg patient body weight per day which can be administered in single or multiple doses. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are may be provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, or may be administered once or twice per day.

When treating, preventing, controlling, ameliorating, or reducing the risk of headache, migraine, cluster headache, or other diseases for which compounds of the present invention are indicated, generally satisfactory results are obtained when the compounds of the present invention are administered at a daily dosage of from about 0.1 milligram to about 100 milligram per kilogram of animal body weight, given as a single daily dose or in divided doses two to six times a day, or in sustained release form. For most large mammals, the total daily dosage is from about 1.0 milligrams to about 1000 milligrams, or from about 1 milligrams to about 50 milligrams. In the case of a 70 kg adult human, the total daily dose will generally be from about 7 milligrams to about 350 milligrams. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Several methods for preparing the compounds of this invention are illustrated in the following Schemes and Examples. Starting materials are made according to procedures known in the art or as illustrated herein.

The compounds of the present invention can be prepared readily according to the following Schemes and specific examples, or modifications thereof, using readily available starting materials, reagents and conventional synthesis procedures. In these reactions, it is also possible to make use of variants which are themselves known to those of ordinary skill in this art but are not mentioned in greater detail. The general procedures for making the compounds claimed in this invention can be readily understood and appreciated by one skilled in the art from viewing the following Schemes.

The synthesis of some heterocyclic amine intermediates may be conducted as described in Schemes 1-3. Related intermediates bearing a variety of substituents may be prepared by employing appropriately substituted starting materials or by derivatization of any intermediates and/or final products as desired by methods known in the art. Other intermediates of interest are described in a number of publications including, but not limited to, the following: Chaturvedula et al. WO 2006/052378; Bell et al. WO 2004/082605; Bell et al. WO 2004/082678; Bell et al. WO 2006/031513; Bell et al. WO 2006/031606; Bell et al. WO 2006/031610.

A representative synthesis of a spiroazaoxindole intermediate is shown in Scheme 1. The known pyridine diester 1 (Hashimoto et al., Heterocycles 1997, 46, 581) may be reduced to the corresponding diol 2 with lithium borohydride. This diol can be converted to the dibromide 3 by reaction with phosphorus tribromide in THF. 7-Azaindole (4) may be protected with a variety of protecting groups, such as the 2-(trimethylsilyl)ethoxymethyl group shown in Scheme 1. Following the method of Marfat and Carter (Tetrahedron Lett, 1987, 28, 4027), treatment of 5 with pyridine hydrobromide perbromide provides the dibromoazaoxindole 6, which may be reduced to the corresponding azaoxindole 7 by reaction with zinc. The key alkylation of 7 with dibromide 3 is carried out using cesium carbonate in DMF to afford the spiroazaoxindole 8. A variety of other bases and solvents may be employed in this alkylation reaction, and use of a different alkylating agent than the dibromide shown here can lead to other products. Treatment of compound 8 with aqueous HCl at reflux effects simultaneous hydrolysis of the nitrile and deprotection of the azaoxindole, affording the key acid intermediate 9. This carboxylic acid may be subjected to known Curtius rearrangement conditions to provide, after deprotection, aminopyridine 10. The methodology shown in Scheme 1 is not limited to azaoxindoles such as 7, but may be applied to a variety of suitably protected heterocyclic systems to give the corresponding spiro compounds.

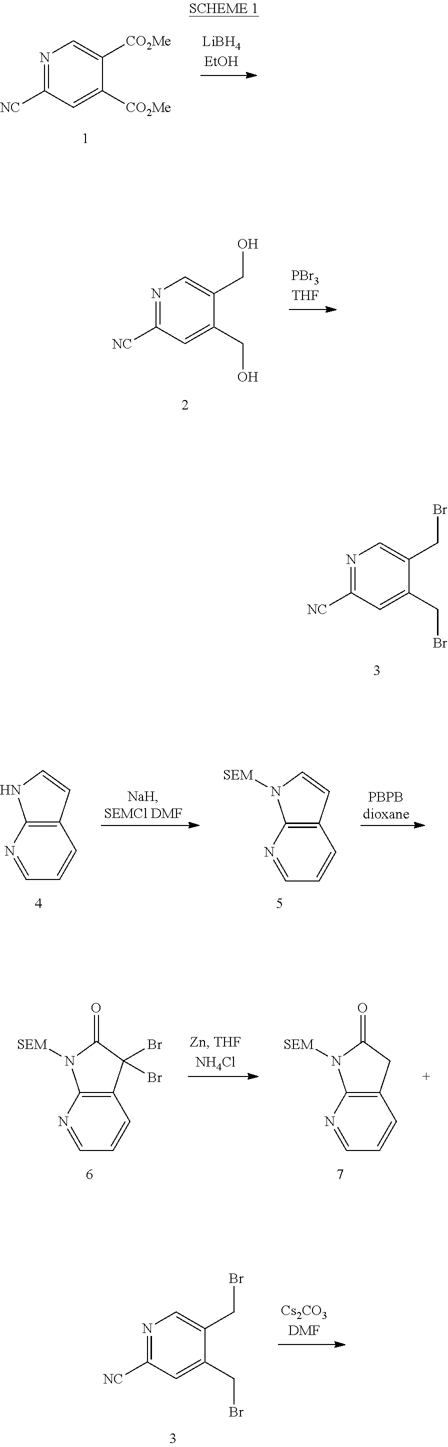

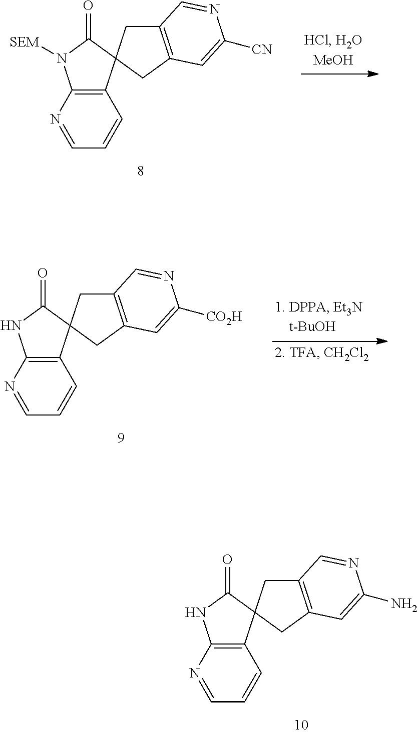

Scheme 2 illustrates a route to an isomer of compound 10, the 3-aminopyridine 15. Bis-alkylation of the spiroazaoxindole 7 with 1,4-dibromobutan-2-one (de Meijere et al., Eur. J. Org. Chem. 2001, 3789) provides the cyclopentanone 11. Condensation of ketone 11 with ammonia and 1-methyl-3,5-dinitropyridin-2(1H)-one (Tohda et al., Bull. Chem. Soc. Japan 1990, 63, 2820) in refluxing methanol leads to the 3-nitropyridine derivative 13. Catalytic hydrogenation may be used to provide the corresponding aniline 14. Standard deprotection of 14 using sequential acid and base treatments affords the 3-aminopyridine intermediate 15.

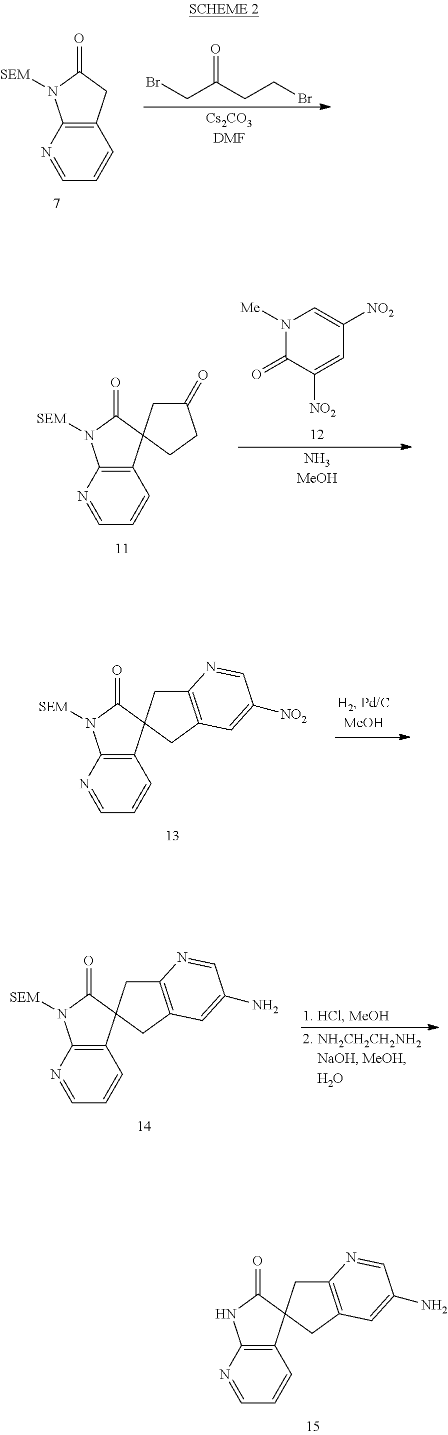

The synthesis of an important intermediate 17 is outlined in Schem 3. The key alkylation of 7 with 1,2-bis(bromomethyl)-4-nitrobenzene (18, Cava et al., J. Org. Chem. 2000, 65, 5413-5415) is carried out using cesium carbonate in DMF to afford the spiroazaoxindole 19. A variety of other bases and solvents may be employed in this alkylation reaction, and use of an alternative alkylating agent to the dibromide shown here can lead to different products. Reduction of the nitro compound 19, for example using hydrogenation over palladium, and a two-step deprotection affords the corresponding aniline 21. The methodology shown in Scheme 3 is not limited to azaoxindoles such as 17, but may be applied to a variety of suitably protected heterocyclic systems to give the corresponding spiro compounds.

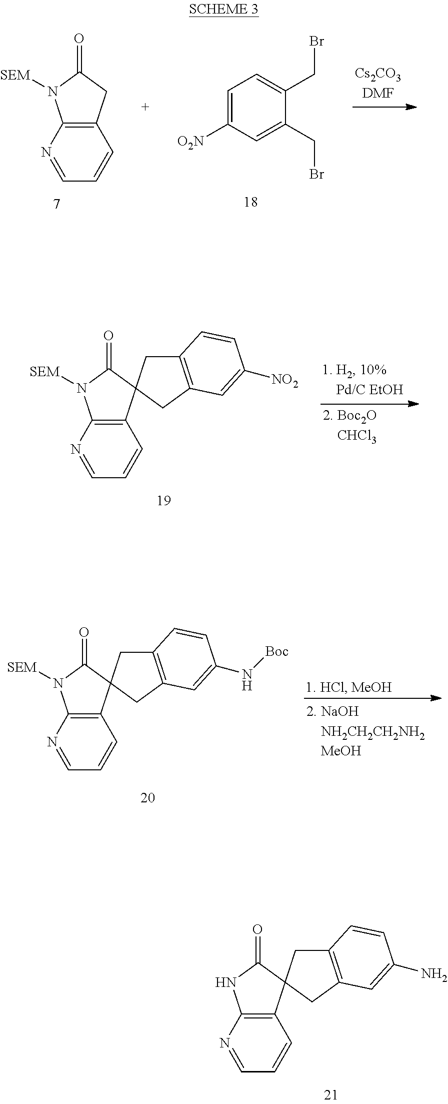

Spiroazaoxindole intermediates, such as those illustrated in Scheme 3, may be resolved to give pure enantiomers using techniques familiar to those skilled in the art. For example, chromatography of the protected intermediate 20 on a ChiralPak AD column can be used to provide the individual enantiomers (+)-20 and (−)-20, and these enantiomers may be converted to the corresponding anilines [(+)-21 and (−)-21] by the two-step deprotection. In the case of compound 21, the dextro isomer is the (R)-enantiomer and the levo isomer (5)-enantiomer, i.e. (+)-21 is (R)-21 and (−)-21 is (S)-21. Use of standard coupling procedures using enantiomerically pure anilines can provide the individual enantiomers of the final products. Resolution may be effected by other methodologies, such as fractional crystallization of diastereomeric salts, and it may be carried out on other synthetic intermediates or on the final Spiroazaoxindole intermediates, such as those illustrated in Schemes 1 and 2, may be resolved to give pure enantiomers using techniques familiar to those skilled in the art. For example, chromatography of the suitable intermediates on a chiral column can be used to provide the individual stereoisomers. Resolution may also be effected by other methodologies, such as fractional crystallization of diastereomeric salts, and it may be carried out on other synthetic intermediates or on the final products. Alternatively, an asymmetric synthesis of a key intermediate could be used to provide an enantiomerically enriched final product.

Imidazolinones similar to 28 can be prepared according to Scheme 4. Using standard chemistry, aminoacids 22 can be converted to amino amides 23 and then to acylated aminoamides 25 by coupling carboxylic acids 24 using procedures well known to those skilled in the art. Cyclization to 26 is accomplished by heating 25 in methanol in the presence of sodium hydroxide. Conversion to intermediates 28 is done in two steps by first alkylating with bromo tert butyl acetate to give 27 followed by acid catalyzed deprotection.

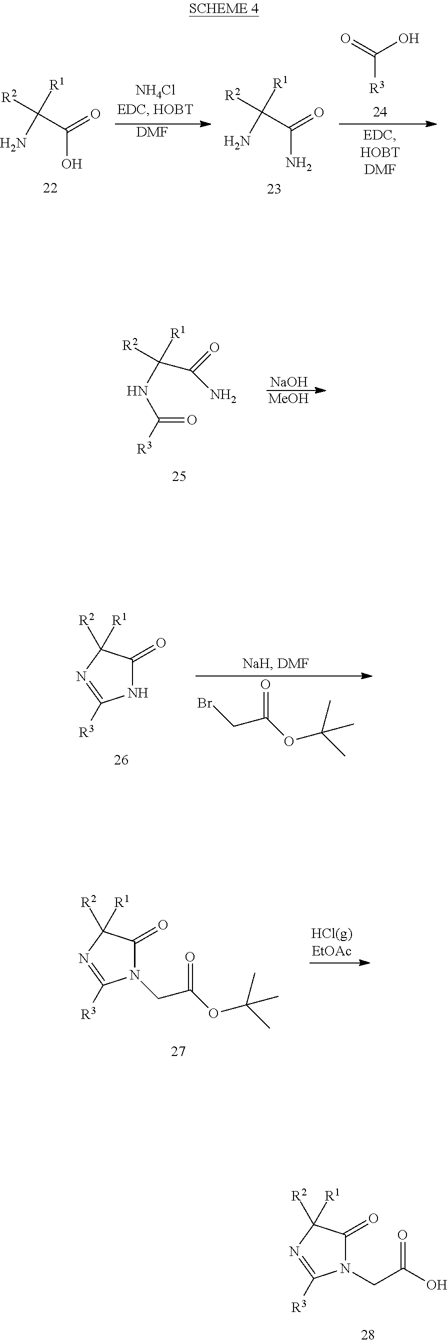

Scheme 5 illustrates the synthesis of the isomeric imidazolinone intermediates 36. Amino amides such as 31 are conveniently prepared by treatment of aminoesters 30 with ammonium hydroxide. Heating 31 with carbonyl compounds in MeOH under acid catalysis provides imidazolidines such as 32 which can be oxidized to imidazolinones 33 by treatment with N-bromosuccinamide. Alkylation of 33 with ethyl bromoacetate followed by resolution and hydrolysis gives acids 36.

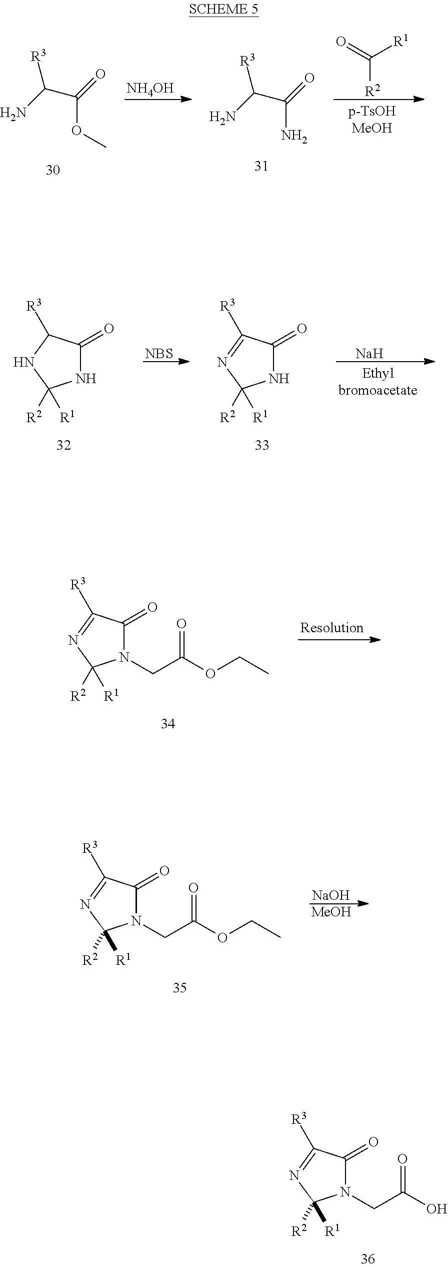

A wide variety of intermediates may be reacted under conditions well known to those skilled in the art to provide the compounds of the present invention. For illustrative purposes, some examples of relevant methodology are shown in Scheme 6.

In Scheme 6, standard coupling of amines (e.g 37) with carboxylic acids (e.g. 28 or 36) may be used to provide compounds of the present invention such as structures 38 and 40. Such coupling reactions may be performed using a variety of known reagents and conditions. Examples include the use of EDC and HOBT in DMF, BOP in DMF, PyBOP in CH₂Cl₂, or HATU in DMF. Alternatively, the carboxylic acid may be activated, for example as the corresponding acid chloride or anhydride, to provide efficient reaction with amines of interest.

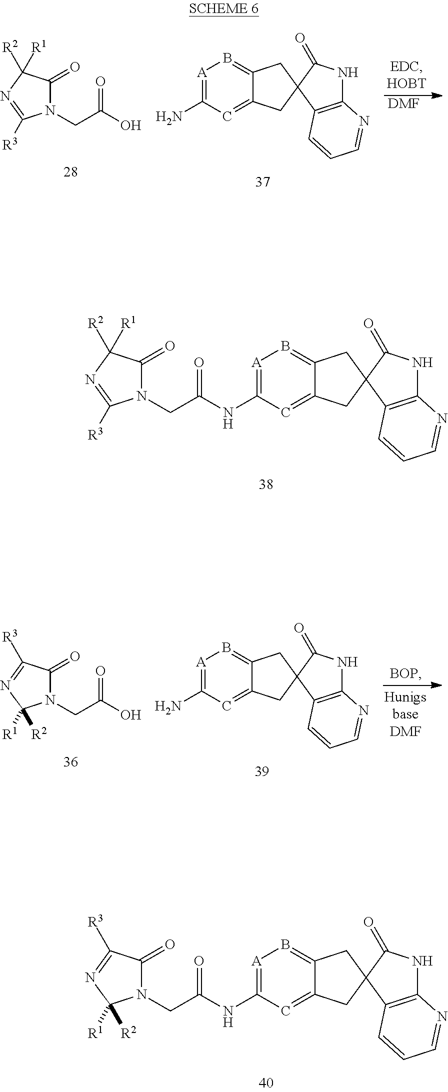

In another example of the synthesis of compounds of the present invention, an olefinic or acetylenic compound can be coupled to an aryl halide under conditions well known to those skilled in the art to generate compounds of the present invention represented by 43, 44 and 46 For example, imidazolinones such as 26 and 33 can be substituted using allyl bromide and sodium hydride to give allylated intermediates such as 41, which can be coupled to aryl halides under the influence of Palladium catalysis using procedures well known to those skilled in the art. The methodology illustrated in Schemes 7, 8 and 9, as well as a wide variety of other transformations known to those skilled in the art of organic synthesis, may be used to synthesize compounds of the present invention.

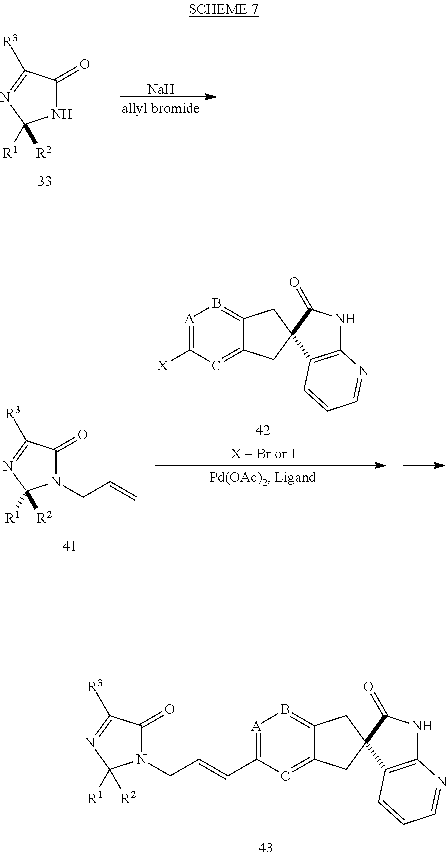

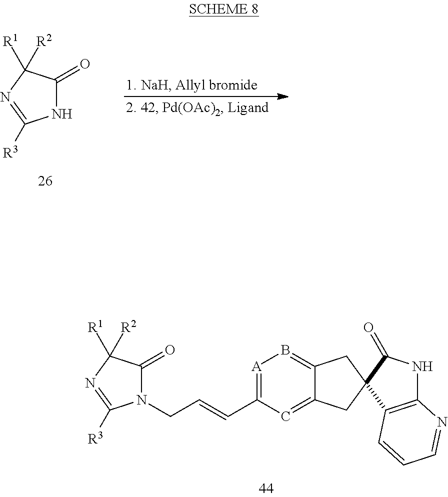

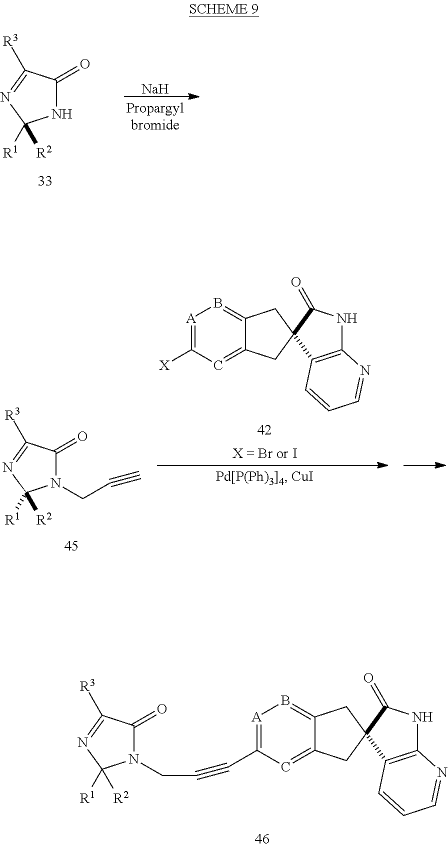

In some cases the final product may be further modified, for example, by manipulation of substituents. These manipulations may include, but are not limited to, reduction, as illustrated in Scheme 10. Other transformations include oxidation, alkylation, acylation, and hydrolysis reactions depending on the substituents, which are commonly known to those skilled in the art.

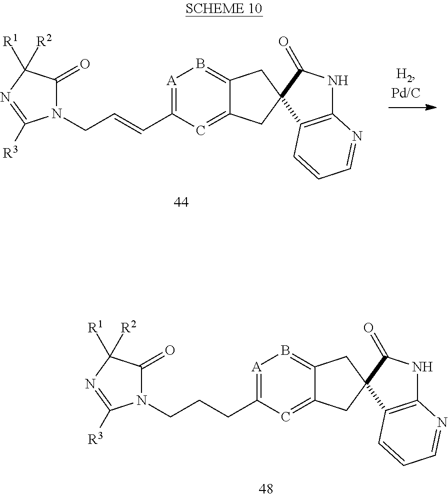

In some cases the order of carrying out the foregoing reaction schemes may be varied to facilitate the reaction or to avoid unwanted reaction products. Additionally, various protecting group strategies may be employed to facilitate the reaction or to avoid unwanted reaction products. The following examples are provided so that the invention might be more fully understood. These examples are illustrative only and should not be construed as limiting the invention in any way.

The racemic intermediates may be resolved to give pure enantiomers using techniques familiar to those skilled in the art. For example, chromatography ChiralPak AD column can be used to provide the individual enantiomers, and these enantiomers may be alkylated and hydrolyzed to the corresponding acid. Use of standard coupling procedures using enantiomerically pure anilines affords the individual enantiomers of the final products. Resolution may be effected by other methodologies, such as fractional crystallization of diastereomeric salts, and it may be carried out on other synthetic intermediates or on the final products. Alternatively, an assymetric synthesis of a key intermediate, such as an amino acid precursor of a spirohydantoin, could be used to provide an enantiomerically enriched final product.

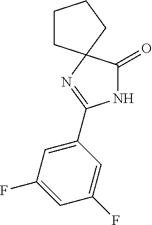

### 2-(3,5-difluorophenyl)-1,3-diazaspiro[4.4]non-1-en-4-one

### Step A. N-[1-(aminocarbonyl)cyclopentyl]-3,5-difluorobenzamide

To a solution of aminocyclopentane carboxamide hydrochloride (300 mg, 1.82 mmol) and Hunigs base (318 uL, 1.82 mmol) in DMF (5 mL) at room temperature was added 1-(3-dimethylaminopropyl)-3-ethyl carbodiimide hydrochloride (349 mg, 1.82 mmol), 1-hydroxybenzotriazole hydrate (279 mg (1.82 mmol) and 3,5 difluorobenzoic acid (288 mg, 1.82 mmole). The mixture was stirred at ambient temperature until HPLC analysis indicated reaction completion. The mixture was diluted with a solution of saturated sodium bicarbonate (50 mL), then extracted with EtOAc (2×50 mL). The organic layer was washed with brine (50 mL), then dried over Na₂SO₄, filtered, and concentrated in vacuo. MS: m/z=269 (M+1).

### Step B. 2-(3,5-difluorophenyl)-1,3-diazaspiro[4.4]non-1-en-4-one

To a solution N-[1-(aminocarbonyl)cyclopentyl]-3,5-difluorobenzamide from Step A (200 mg, 0.746 mmole) in methanol (10 mL) at room temperature was added a solution of sodium hydroxide in water (0.746 mL of 5N solution, 3.7 mmol). The mixture was stirred at ambient temperature for 10 minutes and then heated to 60° C. for 10 minutes. After cooling, the mixture was diluted with water (100 mL), then extracted with EtOAc (2×100 mL). The combined organic layers were washed with brine (50 mL), then dried over Na₂SO₄, filtered, and concentrated in vacuo. MS: m/z=251 (M+1).

### Intermediate 2

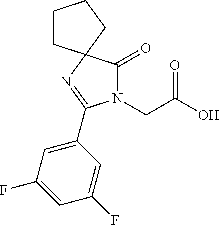

### Step A. tert-butyl [2-(3,5-difluorophenyl)-4-oxo-1,3-diazaspiro[4.4]non-1-en-3-yl]acetate

To a solution of 2-(3,5-difluorophenyl)-1,3-diazaspiro[4.4]non-1-en-4-one from Step B above (150 mg, 0.6 mmol) in DMF (5 mL) was added sodium hydride (60% dispersion in mineral oil, 24 mg, 0.6 mmol) and after 5 minutes tert-butylbromoacetate (117 mg, 90 uL, 0.6 mmol) was added and the reaction stirred at ambient temperature. The mixture was diluted with saturated sodium bicarbonate (100 mL), and then extracted with EtOAc (2×100 mL). The combined organic layers were washed with brine (50 mL), then dried over Na₂SO₄, filtered, and concentrated in vacuo. The crude product was purified by silica gel chromatography, eluting with a gradient of hexane:EtOAc from 80:20 to 40:60, to give the title compound. MS: m/z=365 (M+1).

### Step B. [2-(3,5-difluorophenyl)-4-oxo-1,3-diazaspiro[4.4]non-1-en-3-yl]acetic acid hydrochloride

A solution of tert-butyl [2-(3,5-difluorophenyl)-4-oxo-1,3-diazaspiro[4.4]non-1-en-3-yl]acetate from Step C above (200 mg, 0.55 mmol) in ethyl acetate (50 mL) at 0° C. was saturated with HCl(g) and then allowed to warm to ambient temperature over 1 h. The reaction was concentrated and the material thus obtained was dried overnight under high vacuum to give the product. MS: m/z=309 (M+1).

### Intermediate 3

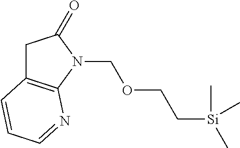

### 1-{[2-(Trimethylsilyl)ethoxy]methyl}-1,3-dihydro-2H-pyrrolo[2,3-b]pyridin-2-one

### Step A. 1-{[2-(Trimethylsilyl)ethoxy]methyl}-1H-pyrrolo[2,3-b]pyridine

Sodium hydride (60% dispersion in mineral oil; 16.2 g, 0.404 mol) was added in portions over 25 min to a solution of 7-azaindole (39.8 g, 0.337 mol) in DMF (200 mL) at 0° C. and the mixture was stirred for 1 h. 2-(Trimethylsilyl)ethoxymethyl chloride (71.8 mL, 0.404 mol) was then added slowly over 15 min, keeping the temperature of the reaction mixture below 10° C. After 1 h, the reaction was quenched with water (500 mL) and the mixture was extracted with CH₂Cl₂ (5×300 mL). The combined organic layers were washed with saturated brine, dried over MgSO₄, filtered, concentrated and dried under high vacuum to give the title compound. MS: m/z=249 (M+1).

### Step B. 3,3-Dibromo-1-{[2-(trimethylsilyl)ethoxy]methyl}-1,3-dihydro-2H-pyrrolo[2,3-b]pyridin-2-one

A solution of 1-{[2-(trimethylsilyl)ethoxy]methyl}-1H-pyrrolo[2,3-b]pyridine from Step A (43.1 g, 0.1735 mol) in dioxane (300 mL) was added dropwise over 30 min to a suspension of pyridine hydrobromide perbromide (277 g, 0.8677 mol) in dioxane (300 mL). The reaction was stirred at ambient temperature using an overhead mechanical stirrer to produce two layers. After 60 min, the reaction was quenched with water (300 mL) and extracted with EtOAc (500 mL). The aqueous layer was extracted further with EtOAc (2×300 mL) and the combined organic layers were washed with H₂O (4×300 mL; the final wash was pH 5-6), then brine (300 mL), dried over MgSO₄, filtered and concentrated in vacuo. The crude product was immediately dissolved in CH₂Cl₂ and the solution filtered through a plug of silica, eluting with CH₂Cl₂ until the dark red color had completely eluted from the plug. The filtrate was washed with saturated aqueous NaHCO₃ (400 mL), then brine (400 mL), dried over MgSO₄ filtered, and concentrated in vacuo to give the title compound. MS: m/z=423 (M+1).

### Step C. 1-{[2-(Trimethylsilyl)ethoxy]methyl}-1,3-dihydro-2H-polo 2,3-b]pyridin-2-one

Zinc (100 g, 1.54 mol) was added to a solution of 3,3-dibromo-1-{[2-(trimethylsilyl)ethoxy]methyl}-1,3-dihydro-2H-pyrrolo[2,3-b]pyridin-2-one (65 g, 0.154 mol) in THF (880 mL) and saturated aqueous NH₄Cl (220 mL). After 3 h, the reaction mixture was filtered and concentrated in vacuo. The residue was partitioned between EtOAc and H₂O which resulted in the formation of a white precipitate. Both layers were filtered through a Celite pad and the layers were separated. The aqueous layer was washed with EtOAc (2×500 mL) and the combined organic layers were washed with H₂O, dried over MgSO₄, filtered, and concentrated under reduced pressure. The crude product was purified by silica gel chromatography, eluting with CH₂Cl₂:EtOAc—90:10, to give the title compound. MS: m/z=265 (M+1).

### Intermediate 4

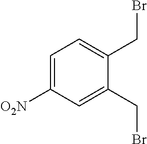

### 1,2-Bis(bromomethyl)-4-nitrobenzene

### Step A. (4-Nitro-1,2-phenylene)dimethanol

A solution of 4-nitrophthalic acid (40 g, 189.5 mmol) in THF (500 mL) was added dropwise over 1.5 h to a solution of borane-THF complex (1 M, 490 mL, 490 mmol), keeping the reaction temperature between 0° C. and 5° C. After the addition, the reaction mixture was allowed to warm slowly to ambient temperature and stirred for 18 h MeOH (100 mL) was added carefully and the precipitated solid dissolved. The mixture was concentrated in vacuo to about 500 mL, cooled to 0° C., and 10 N NaOH was added to adjust the pH to 10-11. This mixture was extracted with EtOAc (3×600 mL) and the combined organic layers were washed with brine, dried over Na₂SO₄, filtered, and concentrated in vacuo to give the title compound. MS: m/z=207 (M−OH+CH₃CN).

### Step B. 1,2-Bis(bromomethyl)-4-nitrobenzene

Phosphorus tribromide (20.1 mL, 212 mmol) in Et₂O (250 mL) was added dropwise over 1.5 h to a solution of (4-nitro-1,2-phenylene)dimethanol from Step A (35.3 g, 193 mmol) in Et₂O (750 mL). After 18 h, the reaction mixture was cooled to 0° C. and quenched with H₂O (100 mL). The layers were separated and the organic layer was washed with H₂O (2×200 mL), then saturated aqueous NaHCO₃, dried over Na₂SO₄, filtered, and concentrated in vacuo to give the title compound. MS: m/z=309 (M+1).

### Intermediate 5

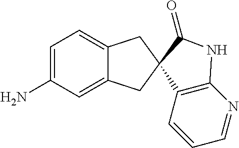

### (R)-5-Amino-3-dihydrospiro[indene-2,3′-pyrrolo[2,3-b]pyridin]-2′(1H)-one

### Step A. (±)-5-Nitro-{[2-(trimethylsilyl)ethoxy]methyl}-1,3-dihydrospiro[indene-2,3′-pyrrolo[2,3-b]pyridin]-2′(1′H)-one

To a solution of 1,2-bis(bromomethyl)-4-nitrobenzene (40.9 g, 132 mmol, described in Intermediate 3) and 1-{[2-(trimethylsilyl)ethoxy]methyl}-1,3-dihydro-2H-pyrrolo[2,3-b]pyridin-2-one (31.5 g, 119 mmol, described in Intermediate 2) in DMF (2 L) was added cesium carbonate (129 g, 397 mmol), portionwise, over 5 min. After 18 h, acetic acid (7.6 mL) was added and the mixture was concentrated to a volume of about 500 mL, then partitioned between EtOAc (1.5 L) and H₂O (1 L). The organic layer was washed with H₂O (1 L), then brine (500 mL), then dried over Na₂SO₄, filtered, and concentrated in vacuo. The crude product was purified by silica gel chromatography, eluting with a gradient of hexane:EtOAc from 100:0 to 0:100, to give the title compound. MS: m/z=412 (M+1).

### Step B. (±)-5-Amino-1′-{[2-(trimethylsilyl)ethoxy])methyl}-1,3-dihydrospiro[indene-2,3′-pyrrolo[2,3-b]pyridin]-2′(1′H)-one

A mixture of 10% Pd/C (3 g) and (±)-5-nitro-1′-{[2-(trimethylsilyl)ethoxy]methyl}-1,3-dihydrospiro[indene-2,3′-pyrrolo[2,3-b]pyridin]-2′(1′H)-one from Step A (19.1 g, 46.4 mmol) was stirred vigorously in EtOH (400 mL) under an atmosphere of hydrogen (ca. 1 atm). After 18 h, the mixture was filtered through a pad of Celite, washing extensively with MeOH, and the filtrate was concentrated in vacuo to give the title compound. MS: m/z=382 (M+1).

### Step C. tert-Butyl (R)-(2′-oxo-1′-{[2-(trimethylsilyl)ethoxy]methyl}-1,1′,2′,3-tetrahydrospiro[indene-2,3′-pyrrolo[2,3-b]pyridin-5-yl)carbamate

A solution of (±)-5-amino-1′-{[2-(trimethylsilyl)ethoxy]methyl}-1,3-dihydrospiro[indene-2,3′-pyrrolo[2,3-b]pyridin]-2′(1′H)-one from Step B (104 g, 273 mmol) and di-tert-butyl dicarbonate (71.5 g, 328 mmol) in CHCl₃ (1 L) was heated to reflux for 17 h. The cooled mixture was concentrated in vacuo and the residue was purified by silica gel chromatography, eluting with hexane:EtOAc from 100:0 to 50:50, to give the racemic product. The enantiomers were resolved by HPLC, utilizing a ChiralPak AD column and eluting with EtOH. The first major peak to elute was tert-butyl (S)-(2′-oxo-1′-{[2-(trimethylsilyl)ethoxy]methyl}-1,1′,2′,3-tetrahydrospiro[indene-2,3′-pyrrolo[2, 3-1)]pyridin]-5-yl)carbamate, and the second major peak to elute was tert-butyl (R)-(2′-oxo-1′-{[2-(trimethylsilyl)ethoxy]methyl}-1,1′,2′,3-tetrahydrospiro[indene-2,3′-pyrrolo[2,3-b]pyridin]-5-yl)carbamate, the title compound. MS: m/z=482 (M+1).

### Step D. (R)-5-Amino-1,3-dihydrospiro[indene-2,3′-pyrrolo[2,3-b]pyridin]-2′(1′H)-one

A solution of tert-butyl (R)-(2′-oxo-1′-{[2-(trimethylsilyl)ethoxy])methyl}-1,1′,2′,3-tetrahydrospiro[indene-2,3′-pyrrolo[2,3-b]pyridin]-5-yl)carbamate from Step C (13.4 g, 27.8 mmol) in MeOH (300 mL) was saturated with HCl (g). The mixture was resaturated with HCl (g) every 30 min until the starting material was consumed, and then concentrated in vacuo. The residue was dissolved in MeOH (150 mL) and treated with ethylenediamine (1.9 mL, 27.8 mmol) and 10 N sodium hydroxide (6 mL, 60 mmol) to adjust the mixture to pH 10. After 30 min, the mixture was diluted with H₂O (400 mL) and extracted with CHCl₃ (1 L). The organic layer was dried over Na₂SO₄, filtered, and concentrated in vacuo. The crude material was triturated with MeOH (35 mL) to give the title compound. MS: m/z 252 (M+1).

### Intermediate 6

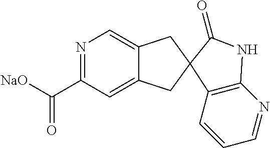

### (±)-Sodium 2′-oxo-1′,2′,5,7-tetrahydrospiro[cyclopenta[c]pyridine-6,3′-pyrrolo[2,3-b]pyridine]-3-carboxylate

### Step A. 4,5-Bis(hydroxymethyl)pyridine-2-carbonitrile

To a solution of dimethyl 6-cyanopyridine-3,4-dicarboxylate (2.00 g, 9.08 mmol) (Hashimoto et al., Heterocycles 1997, 46, 581) in EtOH (50 mL) was added lithium borohydride (4.54 mL of a 2 M solution in THF, 9.08 mmol) dropwise. The reaction mixture was stirred at ambient temperature for 3 h, and then cooled to 0° C. Saturated aqueous NaHCO₃ (20 mL) was added slowly and the quenched mixture was extracted with EtOAc (9×100 mL). The combined organic extracts were dried over Na₂SO₄, filtered, and concentrated in vacuo. The crude product was purified by silica gel chromatography, eluting with a gradient of CH₂Cl₂:MeOH from 100:0 to 85:15, to give the title compound. MS: m/z=165 (M+1).

### Step B. 4,5-Bis(bromomethyl)pyridine-2-carbonitrile

To a solution of 4,5-bis(hydroxymethyl)pyridine-2-carbonitrile from Step A (750 mg, 4.57 mmol) in THF (15 mL) was added phosphorus tribromide (1.61 g, 5.94 mmol) in THF (5 mL) dropwise. The reaction mixture was stirred at ambient temperature for 2 h, and then cooled to 0° C. Saturated aqueous NaHCO₃ (5 mL) was added slowly and the quenched mixture was extracted with CHCl₃ (2×30 mL). The combined organic extracts were dried over Na₂SO₄, filtered, and concentrated in vacuo. The crude product was purified by silica gel chromatography, eluting with a gradient of hexane:EtOAc from 100:0 to 25:75, to give the title compound. MS: m/z=291 (M+1).

### Step C. (±)-2′-Oxo-1′,2′,5,7-tetrahydrospiro[cyclopenta[c]pyridine-6,3′-pyrrolo[2,3-b]pyridine]-3-carbonitrile

To a solution of 4,5-bis(bromomethyl)pyridine-2-carbonitrile from Step B (2.56 g, 8.83 mmol) and 1,3-dihydro-2H-pyrrolo[2,3-b]pyridin-2-one (Marfat & Carta, Tetrahedron Lett., 1987, 28, 4027) (1.18 g, 8.83 mmol) in THF (120 mL) and H₂O (60 mL) was added lithium hydroxide monohydrate (1.11 g, 26.5 mmol). After 20 min, the reaction mixture was poured onto water (100 mL) and extracted with EtOAc (3×100 mL). The combined organic extracts were dried over Na₂SO₄, filtered, and concentrated in vacuo. The crude product was purified by silica gel chromatography, eluting with a gradient of CH₂Cl₂:MeOH: NH₄OH from 100:0:0 to 95:5:1, to give the title compound. MS: m/z=263 (M+1).

### Step D. (±)-Sodium 2′-oxo-1′,2′,5,7-tetrahydrospiro[cyclopenta[c]pyridine-6,3′-pyrrolo[2,3-b]pyridine]-3-carboxylate

To a solution of (±)-2′-oxo-1′,2′,5,7-tetrahydrospiro[cyclopenta[c]pyridine-6,3′-pyrrolo[2,3-b]pyridine]-3-carbonitrile from Step C (1.53 g, 5.83 mmol) in EtOH (20 mL) was added 5 M aqueous NaOH (3.50 mL). The mixture was heated at reflux for 72 h, with additional 5 M aqueous NaOH (2.00 mL) added at 6 h. The reaction mixture was allowed to cool and was concentrated to dryness in vacuo to afford the title compound in sufficient purity for use in subsequent steps. MS: nil z=282 (M+1).

### Intermediate 7

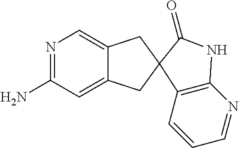

### (±)-3-Amino-5,7-dihydrospiro[cyclopenta[c]pyridine-6,3′-pyrrolo[2,3-b]pyridin]-2′(1′H)-one

### Step A. (±)-tert-Butyl (2′-oxo-1′,2′, 5,7-tetrahydrospiro[cyclopenta[c]pyridine-6,3′-pyrrolo[2,3-b]pyridin]-3-yl)carbamate

To a suspension of (±)-sodium 2′-oxo-1′,2′,5,7-tetrahydrospiro[cyclopenta[c]pyridine-6,3′-pyrrolo[2,3-b]pyridine]-3-carboxylate (1.64 g, 5.83 mmol, described in Intermediate 2) and triethylamine (1.62 mL, 11.7 mmol) in tert-butanol (50 mL) was added diphenylphosphoryl azide (1.89 mL, 8.75 mmol) and the mixture was heated at reflux for 72 h. Additional diphenylphosphoryl azide (1.89 mL, 8.75 mmol) was added after 24 h and 56 h. The reaction mixture was concentrated in vacuo and then partitioned between CH₂Cl₂ (75 mL) and saturated NaHCO₃ (100 mL). The organic layer was separated and the aqueous layer was further extracted with CH₂Cl₂ (2×50 mL). The combined organic layers were dried over Na₂SO₄, filtered, and concentrated in vacuo. The crude product was purified by silica gel chromatography, eluting with a gradient of CH₂Cl₂:MeOH: NH₄OH from 100:0:0 to 95:5:1, to give the title compound. MS: m/z=353 (M+1).

### Step B. (±)-3-Amino-5,7-dihydrospiro[cyclopenta[c]pyridine-6,3′-pyrrolo[2,3-b]pyridin]-2′(1′H)-one trifluoroacetate

A solution of (±)-tert-butyl (2′-oxo-1′,2′,5,7-tetrahydrospiro[cyclopenta[c]pyridine-6,3′-pyrrolo[2,3-b]pyridin]-3-yl)carbamate from Step A (131 mg, 0.372 mmol) was stirred in CH₂Cl₂ (10 mL) and TFA (3 mL) for 18 h and then concentrated in vacuo to provide the title compound. MS: m/z=253 (M+1).

### Intermediate 8

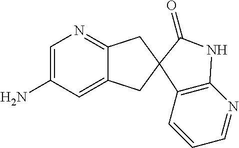

### (±)-3-Amino-5,7-dihydrospiro[cyclopenta[b]pyridine-6,3′-pyrrolo[2,3-b]pyridin]-2′(1′H)-one trifluoroacetate

### Step A. (±)-1′-{[2-(Trimethylsilyl)ethoxy]methyl}-3H-spiro[cyclopentane-1,3′-pyrrolo[2,3-b]pyridine]-2′,3(1′H)-dione

To a solution of 1-{[2-(trimethylsilyl)ethoxy]methyl}-1,3-dihydro-2,1-pyrrolo[2,3-b]pyridin-2-one (2.50 g, 9.46 mmol) and cesium carbonate (6.78 g, 20.8 mmol) in DMF (45 mL) was added dropwise a solution of 1,4-dibromobutan-2-one (1.59 mL, 12.3 mmol) (de Meijere et al., Eur. J. Org. Chem., 2001, 3789) in DMF (45 mL). After 68 h, the mixture was partitioned between Et₂O (200 mL) and H₂O (200 mL). The organic layer was separated and the aqueous layer was further extracted with Et₂O (2×100 mL). The combined organic layers were dried over Na₂SO₄, filtered, and concentrated in vacuo. The crude product was purified by silica gel chromatography, eluting with a gradient of hexane:EtOAc from 100:0 to 75:25, to give the title compound. MS: m/z=333 (M+1).

### Step B. (±)-3-Nitro-1′-{[2-(trimethylsilyl)ethoxy]methyl}-5,7-dihydrospiro[cyclopenta[b]pyridine-6,3′-pyrrolo [2,3-b]pyridin]-2′(1′H)-one

A mixture of (±)-1′-{[2-(trimethylsilyl)ethoxy]methyl}-3H-spiro[cyclopentane-1,3′-pyrrolo[2,3-b]pyridine]-2′,3(1′H)-dione from Step A (230 mg, 0.692 mmol) and 1-methyl-3,5-dinitropyridin-2(1H)-one (173 mg, 0.869 mmol) (Tohda et al., Bull. Chem. Soc. Japan, 1990, 63, 2820) in 2 M ammonia in MeOH (3.5 mL) was heated to reflux for 18 h. The mixture was concentrated in vacuo and purified by silica gel chromatography, eluting with a gradient of hexane:EtOAc from 100:0 to 50:50, to give the title compound. MS: m/z=413 (M+1).

### Step C. (±)-3-Amino-1′-{[2-(trimethylsilyl)ethoxy]methyl}-5,7-dihydrospiro[cyclopenta[b]pyridine-6,3′-pyrrolo[2,3-b]pyridin]-2′(1′H)-one

A mixture of 10% Pd/C (20 mg) and (±)-3-nitro-1′-{[2-(trimethylsilyl)ethoxy])methyl}-5,7-dihydro spiro[cyclopenta[b]pyridine-6,3′-pyrrolo[2,3-b]pyridin]-2′(1′H)-one from Step B (117 mg, 0.284 mmol) was stirred vigorously in MeOH (5 mL) under an atmosphere of hydrogen (ca. 1 atm). After 4.5 h, the mixture was filtered through a pad of Celite, washing extensively with MeOH, and the filtrate was concentrated in vacuo to give the title compound. MS: m/z=383 (M+1).

### Step D. (±)-3-Amino-5,7-dihydrospiro[cyclopenta[b]pyridine-6,3′-pyrrolo[2,3-b]pyridin]-2′(1′H)-one trifluoroacetate

A solution of (±)-3-amino-1′-{[2-(trimethylsilyl)ethoxy]methyl}-5,7-dihydrospiro[cyclopenta[b]pyridine-6,3′-pyrrolo[2,3-b]pyridin]-2′(1′H)-one from Step C (117 mg, 0.306 mmol) in MeOH (5 mL) was saturated with HCl (g). The mixture was stirred for 30 min and then concentrated in vacuo. The residue was dissolved in MeOH (3 mL), treated with ethylenediamine (0.020 mL, 0.306 mmol), and 10 N sodium hydroxide was added to adjust the mixture to pH 10. After 1 h, the reaction mixture was purified directly by HPLC using a reversed phase C18 column and eluting with a gradient of H₂O:CH₃CN:CF₃CO₂H from 90:10:0.1 to 5:95:0.1. MS: m/z=253 (M+1).

### Intermediate 9

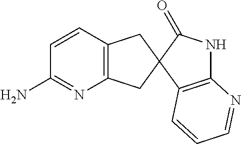

### (±)-2-Amino-5,7-dihydrospiro[cyclopenta[b]pyridine-6,3′-pyrrolo[2,3-b]pyridin]-2′(1′H)-one

### Step A. Dimethyl 6-cyanopyridine-2,3-dicarboxylate

To a solution of dimethylpyridine-2,3-dicarboxylate 1-oxide (Niiyami et al., Bioorg. Med. Chem. Lett., 2002, 12, 3041) (15.3 g, 72.5 mmol) and trimethylsilyl cyanide (15.7 mL, 117 mmol) in DME (161 mL) was added dimethylcarbamoyl chloride (10.5 mL, 114 mmol). The reaction mixture was heated at reflux for 72 h, and then cooled to 0° C. Saturated aqueous NaHCO₃ (800 mL) was added slowly and the quenched mixture was extracted with EtOAc (2×1 L). The combined organic extracts were washed with brine (200 mL), dried over Na₂SO₄, filtered, and concentrated in vacuo. The crude product was purified by silica gel chromatography, eluting with a gradient of hexane:EtOAc from 100:0 to 50:50, to give the title compound. MS: m/z=221 (M+1).

### Step B. 5,6-Bis(hydroxymethyl)pyridine-2-carbonitrile

To a solution of dimethyl 6-cyanopyridine-2,3-dicarboxylate from Step A (13.0 g, 59.0 mmol) in EtOH (295 mL) was added lithium borohydride (29.5 mL of a 2 M solution in THF, 59.0 mmol) dropwise. The reaction mixture was stirred at ambient temperature for 4 h, and then cooled to 0° C. Saturated aqueous NaHCO₃ (200 mL) was added slowly and the quenched mixture was extracted with EtOAc (9×100 mL). The combined organic extracts were dried over Na₂SO₄, filtered, and concentrated in vacuo. The crude product was purified by silica gel chromatography, eluting with a gradient of CH₂Cl₂:MeOH from 100:0 to 85:15, to give the title compound. MS: m/z=165 (M+1).

### Step C. 5,6-Bis(bromomethyl)pyridine-2-carbonitrile

To a solution of 5,6-bis(hydroxymethyl)pyridine-2-carbonitrile from Step B (2.50 g, 15.2 mmol) in THF (76 mL) was added phosphorus tribromide (5.36 g, 19.8 mmol) in THF (20 mL) dropwise. The reaction mixture was stirred at ambient temperature for 2 h, and then cooled to 0° C. Saturated aqueous NaHCO₃ (20 mL) was added slowly and the quenched mixture was extracted with CH₂Cl₂ (2×200 mL). The combined organic extracts were dried over Na₂SO₄, filtered, and concentrated in vacuo. The crude product was purified by silica gel chromatography, eluting with a gradient of hexane:EtOAc from 100:0 to 30:70, to give the title compound. MS: m/z=291 (M+1).

### Step D. (±)-2′-Oxo-1′-{[2-(trimethylsilyl)ethoxy]methyl}-1′,2′,5,7-tetrahydrospiro[cyclopenta[b]pyridine-6,3′-pyrrolo[2,3-b]pyridine]-2-carbonitrile

To a solution of 5,6-bis(bromomethyl)pyridine-2-carbonitrile from Step C (1.80 g, 6.21 mmol) and 1-{[2-(trimethylsilyl)ethoxy]methyl}-1,3-dihydro-2H-pyrrolo[2,3-b]pyridin-2-one (1.64 g, 6.21 mmol, described in Intermediate 1) in DMF (207 mL) was added cesium carbonate (6.07 g, 18.6 mmol), portionwise, over 5 min. After 18 h, the mixture was partitioned between CH₂Cl₂ (100 mL), saturated aqueous NaHCO₃ (100 mL) and brine (200 mL). The organic layer was removed and the aqueous layer was extracted further with CH₂Cl₂ (2×100 mL) The combined organic extracts were dried over Na₂SO₄, filtered, and concentrated in vacuo. The crude product was purified by silica gel chromatography, eluting with a gradient of hexane:EtOAc from 100:0 to 10:90, to give the title compound. MS: m/z=393 (M+1).

### Step E. (±)-2′-Oxo-1′,2′,5,7-tetrahydrospiro[cyclopenta[b]pyridine-6,3′-pyrrolo 2,3-b]pyridine]-2-carboxylic acid

To a solution of (±)-2′-oxo-1′-{[2-(trimethylsilyl)ethoxy]methyl}-1′,2′,5,7-tetrahydrospiro[cyclopenta[b]pyridine-6,3′-pyrrolo[2,3-b]pyridine]-2-carbonitrile from Step D (690 mg, 1.76 mmol) in THF (5 mL) was added 3 N aqueous HCl (36 mL). The mixture was heated at reflux for 18 h, allowed to cool and concentrated to dryness in vacuo. The reaction mixture was dissolved in water (12 mL) and purified directly by HPLC using a reversed phase C18 column and eluting with a gradient of H₂O:CH₃CN:CF₃CO₂H from 95:5:0.1 to 5:95:0.1. Lyophilization of the product-containing fractions provided the title compound. MS: m/z=282 (M+1).

### Step F. (±)-tert-Butyl (2′-oxo-1′,2′,5,7-tetrahydrospiro[cyclopenta[b]pyridine-6,3′-pyrrolo[2,3-b]pyridin]-2-yl)carbamate

To a suspension of (±)-2′-oxo-1′,2′,5,7-tetrahydrospiro[cyclopenta[b]pyridine-6,3′-pyrrolo[2,3-b]pyridine]-2-carboxylic acid from Step E (224 mg, 0.796 mmol) and triethylamine (0.333 mL, 2.39 mmol) in tert-butanol (5 mL) was added diphenylphosphoryl azide (0.258 mL, 1.20 mmol) and the mixture was heated at reflux for 1 h. The reaction mixture was concentrated in vacuo and then partitioned between CH₂Cl₂ (20 mL) and saturated NaHCO₃ (20 mL). The organic layer was separated and the aqueous layer was further extracted with CH₂Cl₂ (2×20 L). The combined organic layers were dried over Na₂SO₄, filtered, and concentrated in vacuo. The crude product was purified by silica gel chromatography, eluting with a gradient of CH₂Cl₂:MeOH: NH₄OH— 100:0:0 to 95:5:1, to give the title compound. MS: m/z=353 (M+1).

### Step G. (±)-2-Amino-5,7-dihydrospiro[cyclopenta[b]pyridine-6,3′-pyrrolo[2,3-b]pyridin]-2′(1′H)-one

A solution of (±)-tert-butyl (2′-oxo-1′,2′,5,7-tetrahydrospiro[cyclopenta[b]pyridine-6,3′-pyrrolo[2,3-b]pyridin]-2-yl)carbamate from Step F (147 mg, 0.417 mmol) was stirred in CH₂Cl₂ (6 mL) and TFA (1 mL) for 3 h and then concentrated in vacuo to provide the title compound as the TFA salt. MS: m/z=253 (M+1).

### Intermediate 10

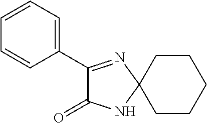

### 3-phenyl-1,4-diazaspiro[4.5]dec-3-en-2-one

### Step A. 2-amino-2-phenylacetamide hydrochloride

To ice-chilled methanol (300 mL) under argon, thionyl chloride (50 mL, 660 mmol) was carefully added dropwise over 30 min. Then, phenylglycine (50 g, 330 mmol) was added. The ice-bath was removed and the reaction mixture was heated to reflux for 16 h. The reaction was then evaporated under reduced pressure. Diethyl ether (200 mL) was added followed by filtration to give the product provided as the hydrochloride salt. ¹H NMR (DMSO-d₆, 400 MHz) δ: 9.24 (s, 3H), 7.49˜7.51 (m, 2H), 7.40˜7.41 (m, 3H), 5.20 (s, 1H), 3.65 (s, 3H).

### Step B. 2-amino-2-phenylacetamide hydrochloride

Phenylglycine methyl ester hydrochloride from Step A. (64 g, 317 mmol) was dissolved in ammonia (150 mL) and stirred at room temperature under argon for 16 h. The reaction mixture was extracted with DCM (200 mL×3), dried over Na₂SO₄ and evaporated under reduced pressure to a white solid which was dried under reduced pressure to afford The product. ¹H NMR: (DMSO-d₆, 400 MHz) δ: 7.50 (s, 1H), 7.19-7.48 (m, 5H), 7.02 (s, 1H), 4.28 (s, 1H).

### Step C. 3-phenyl-1,4-diazaspiro[4.5]decan-2-one

To the product of step B above (0.5 g, 3.3 mmol) in methanol was added cyclohexanone (0.32 g, 3.3 mmol) and H—Y Zeolizes (1.0 g), and the mixture stirred under reflux for 24 h under argon. The reaction was allowed to cool to room temperature and was filtered and the solid washed well with methanol. The filtrate was evaporated to afford the title compound as a white solid, after trituration with hexane (0.52 g, yield: 68%). ¹H NMR (DMSO-d₆, 400 MHz) δ: 8.57 (s, 1H), 7.36˜7.46 (m, 2H), 7.20˜7.33 (m, 3H), 4.55 (d, J=11.6 Hz, 1H), 3.34 (d, J=12 Hz, 1H), 1.35-1.92 (m, 10H).

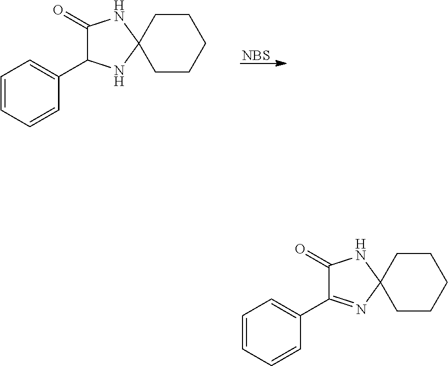

### Step D. 3-phenyl-1,4-diazaspiro[4.5]dec-3-en-2-one

From Step C above (3 g, 13 mmol) was dissolved in DCM and was stirred at room temperature for 16 h under argon atmosphere with N-Bromosuccinimide (2.3 g, 13 mmol). A solution of saturated sodium bicarbonate was added and stirring continued for 1 h at room temperature. The organic layer was separated, dried and evaporated under reduced pressure to yield the product as a white solid after trituration with hexane (2.8 g, yield: 96.5%). ¹H NMR (DMSO-d₆, 400 MHz) δ: 10.20 (s, 1H), 8.28 (t, J=8 Hz, 2H), 7.44-7.52 (m, 3H), 1.44-1.75 (m, 10H).

Employing procedures substantially as described above for Intermediate 10, the following intermediate examples were prepared

TABLE 1

| Intermediate | Structure | NMR |
|---|---|---|
| 10-b | 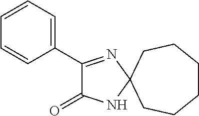 | ¹H NMR (DMSO-d₆, 400 MHz) δ: 8.27 (t, J = 8.4 Hz, 2H), 7.40-7.52 (m, 3H), 1.50-1.89 (m, 12H). |
| 10-c | 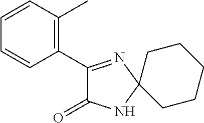 | ¹H NMR (DMSO-d₆, 400 MHz) δ: 7.72-7.75 (m, 1H), 7.33-7.35 (m, 1H), 7.23-7.29 (m, 2H), 2.38 (s, 3H), 1.46-1.75 (m, 10H). |
| 10-e | 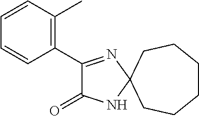 | ¹H NMR (DMSO-d₆, 400 MHz) δ: 7.67-7.69 (m, 1H), 7.15-7.36 (m, 3H), 2.35 (s, 3H), 1.56-1.88 (m, 12H). |
| 10-f | 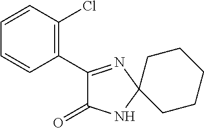 | ¹H NMR (DMSO-d₆, 400 MHz) δ: 10.21 (s, 1H), 7.43-7.61 (m, 4H), 1.46-1.79 (m, 10H). MS (m/z): 263 (M + 1) |
| 10-g | 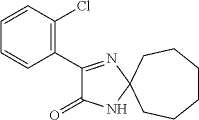 | ¹H NMR (DMSO-d₆, 400 MHz) δ: 10.21 (s, 1H), 7.45-7.59 (m, 4H), 1.90-1.93 (m, 2H), 1.65-1.74 (m, 10H). MS (m/z): 277 (M + 1) |
| 10-h | 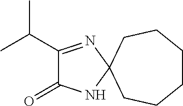 | ¹H NMR (DMSO-d₆, 400 MHz) δ: 9.80 (s, 1H), 2.65-2.75 (m, 1H), 1.35-1.80 (m, 12H), 1.09 (d, J = 6.8 Hz, 6H). |
| 10-i | 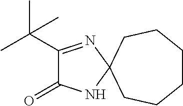 | MS (m/z): 233 (M + 1) |
| 10-j | 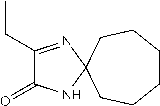 | MS (m/z): 195 (M + 1) |
| 10-k | 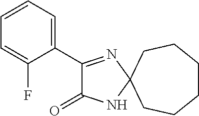 | MS (m/z): 261 (M + 1) |
| 10-l | 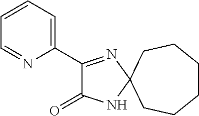 | MS (m/z): 244 (M + 1) |

### Intermediate 11

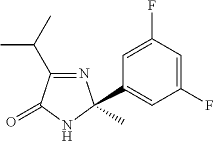

### (2R)-2-(3,5-difluorophenyl)-5-isopropyl-2-methyl-2,3-dihydro-4-H imidazol-4-one

### Step A. 2-(3,5-difluorophenyl)-5-isopropyl-2-methylimidazolidin-4-one

To a solution of 2-amino-3-methylbutanamide (20 g, 172 mmol) in methanol was added 3,5 difluoroacetophenone (26.89 g, 172 mmol) and H—Y zeolites (15 g), and the mixture stirred under reflux for 24 h under argon. The reaction was allowed to cool to room temperature and was filtered and the solid washed well with methanol. The filtrate was evaporated to afford the product as a white solid, after trituration with hexane. ¹H NMR (DMSO-d₆, 400 MHz) δ: 8.93 (s, 1H), 7.05-7.19 (m, 2H), 7.05-7.10 (m, 1H), 3.54-3.56 (m, 1H), 2.21 (s, 1H), 1.68-1.70 (m, 1H), 1.52 (s, 3H), 0.86-0.88 (d, J=8 Hz, 3H), 0.62-0.64 (d, J=8 Hz, 3H),

### Step B. 2-(3,5-difluorophenyl)-5-isopropyl-2-methyl-2,3-dihydro-4-H imidazol-4-one

The product of Step A above (11 g, 43 mmol) was dissolved in DCM and was stirred at room temperature for 16 h under argon atmosphere with N-Bromosuccinimide (7.7 g, 43 mmol). A solution of saturated sodium bicarbonate was added and stirring continued for 1 h at room temperature. The organic layer was separated, dried and evaporated under reduced pressure to yield the racemic compound as a white solid after trituration with hexane.

### Step C (2R)-2-(3,5-difluorophenyl)-5-isopropyl-2-methyl-2,3-dihydro-4-H imidazol-4-one and (2S)-2-(3,5-difluorophenyl)-5-isopropyl-2-methyl-2,3-dihydro-4-H imidazol-4-one

The product of Step B above was separated by chiral SFC (supercritical fluid chromatography) to afford both enantiomers, intermediate 11a (faster eluting enantiomer in chiral SFC) and intermediate 11b (slower eluting enantiomer in chiral SFC).

TABLE 2

| Intermediate | Structure | Characterization |
|---|---|---|
| 11-b | 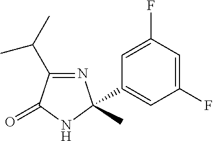 | ¹H NMR (DMSO- d₆, 400 MHz) δ: 10.34 (s, 1H), 7.12- 7.23 (m, 3H), 2.81- 2.90 (m, 1H), 1.63 (s, 1H), 1.14-1.19 (m, 6H), |
| 11-c | 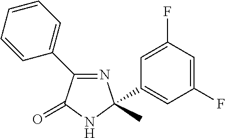 | MS (m/z): 287 (M + 1) |
| 11-d | 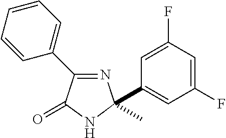 | MS (m/z): 287 (M + 1) |
| 11-e | 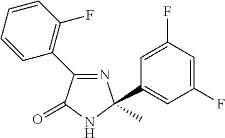 | MS (m/z): 305 (M + 1) |
| 11-f | 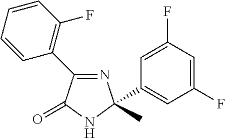 | MS (m/z): 305 (M + 1) |
| 11-g | 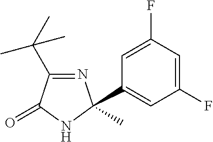 | MS (m/z): 267 (M + 1) |
| 11-h | 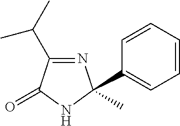 | MS (m/z): 217 (M + 1) |

### Intermediate 12

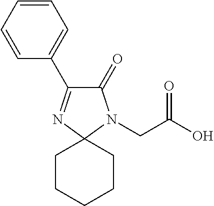

### (2-oxo-3-phenyl-1,4-diazaspiro[4.5]dec-3-en-1-yl)acetic acid

### Step A. ethyl (2-oxo-3-phenyl-1,4-diazaspiro[4.5]dec-3-en-1-yl)acetate

A mixture of compound 3-phenyl-1,4-diazaspiro[4.5]dec-3-en-2-one (2.8 g, 12.3 mmol), ethyl bromoacetate (2 g, 12.3 mmol) and potassium carbonate in DMF was heated at 60° C. for 18 h with rapid stirring under argon atmosphere. After cooling, the reaction solution was poured into water and extracted with ethyl acetate (3×40 mL). The organic layer was separated, washed with saturated brine (40 mL), dried and evaporated. The residue was chromatographed over silica gel to afford the title compound which was used directly in the next step.

### Step B. (2-oxo-3-phenyl-1,4-diazaspiro[4.5]dec-3-en-1-yl)acetic acid

To a stirred mixture of ethyl (2-oxo-3-phenyl-1,4-diazaspiro[4.5]dec-3-en-1-yl)acetate (3.2 g, 10.2 mmol) in water (200 mL) and methanol (100 mL) was added 2N sodium hydroxide solution (6.1 mL, 12.2 mmol). The reaction solution was heated at 60° C. for 16 h, cooled and evaporated under reduced pressure. The residue was partitioned between water and ethyl acetate, the aqueous layer was acidified with 5N HCl and extracted into DCM, the DCM extracts were dried and evaporated under reduced pressure to give the title compound. ¹H NMR (DMSO-d₆, 400 MHz) δ: 8.30-8.33 (m, 2H), 7.48-7.58 (m, 3H), 4.18 (s, 2H), 1.58-2.01 (m, 7H), 1.23-1.37 (m, 3H). MS (m/z): 287 (M+1)

The following intermediate examples in Table 3 were prepared similarly.

TABLE 3

| Intermediate | Structure | Characterization |
|---|---|---|
| 12-b | 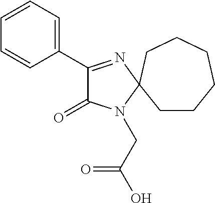 | ¹H NMR (DMSO-d₆, 400 MHz) δ: 8.30-8.33 (m, 2 H), 7.48-7.57 (m, 3 H), 4.21 (s, 2 H), 1.96- 2.08 (m, 2 H), 1.78-1.84 (m, 2 H), 1.51-1.83 (m, 8 H). MS (m/z): 301 (M + 1) |
| 12-c | 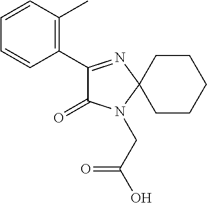 | ¹H NMR (DMSO-d₆, 400 MHz) δ: 12.90 (s, 1 H), 7.86-7.88 (m, 1 H), 7.28-7.38 (m, 3 H), 4.17 (s, 2 H), 2.46 (s, 3 H), 1.98-1.99 (m, 2 H), 1.72- 1.95 (m, 5 H), 1.25-1.29 (m, 3 H). MS (m/z): 301 (M + 1) |
| 12-d | 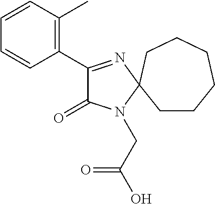 | ¹H NMR (DMSO-d₆, 400 MHz) δ: 12.90 (s, 1 H), 7.81-7.83 (m, 1 H), 7.26-7.38 (m, 3 H), 4.19 (s, 2 H), 2.42 (s, 3 H), 2.04-2.08 (m, 2 H), 1.58- 1.80 (m, 10 H). MS (m/z): 315 (M + 1) |
| 12-e | 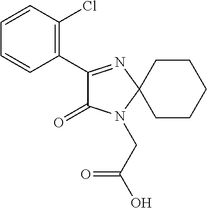 | ¹H NMR (DMSO-d₆, 400 MHz) δ: 7.45-7.65 (m, 4 H), 4.17 (s, 2 H), 2.46-247 (m, 2 H), 1.70- 1.9-1.75 (m, 5 H), 1.28-1.31 (m, 3 H). MS (m/z): 321 (M + 1) |
| 12-f | 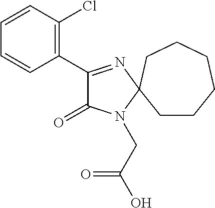 | ¹H NMR (DMSO-d₆, 400 MHz) δ: 7.42-7.62 (m, 4 H), 4.20 (s, 2 H),. 1.97-2.03 (m, 2 H), 1.59- 1.75 (m, 8 H), 1.42-1.48 (m, 2 H). MS (m/z): 335 (M + 1) |
| 12-g | 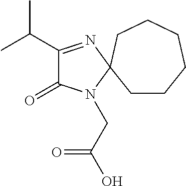 | ¹H NMR (DMSO-d₆, 400 MHz) δ: 12.90 (s, 1 H), 4.15 (s, 2 H), 2.85-2.87 (m, 1 H), 1.94-1.97 (m, 2 H), 1.59-1.75 (m, 8 H), 1.42-1.48 (m, 2 H), 1.10-1.20 (m, 6 H). MS (m/z): 267 (M + 1) |
| 12-h | 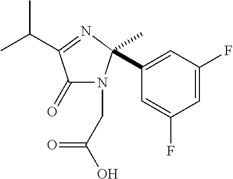 | MS (m/z): 311 (M + 1) |
| 12-i | 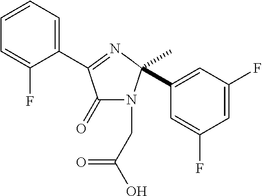 | MS (m/z): 363 (M + 1) |
| 12-j | 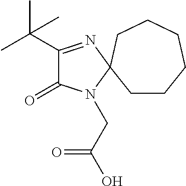 | MS (m/z): 281 (M + 1) |
| 12-k | 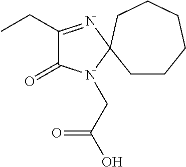 | MS (m/z): 253 (M + 1) |
| 12-l | 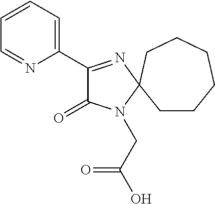 | MS (m/z): 302 (M + 1) |
| 12-m | 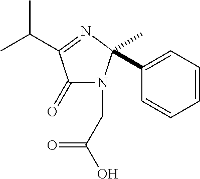 | MS (m/z): 275 (M + 1) |

### Intermediate 13

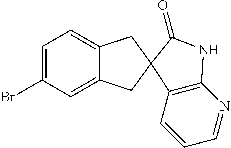

### (R)-5-bromo-1,3-dihydrospiro[indene-2,3′-pyrrolo[2,3-b]pyridin]-2′(1′H)-one

To a cooled 0° C. solution of (R)-5-Amino-1,3-dihydrospiro[indene-2,3′-pyrrolo[2,3-b]pyridin]-2′(1′H)-one (0.5 g 2 mmol) in 4 mL of 48% HBr was added slowly over 10 minutes a solution of sodium nitrite (137 mg, 1.99 mmol) in 0.8 mL (0.6 mL with a 0.2 mL wash) of water. After 5 minutes CuBr (285 mg, 1.99 mole) was added and a condenser with nitrogen flow was attached. The reaction mixture was placed into a 100° C. bath and heated to 100° C. for 20 min. The reaction was then diluted with water to get major precipitation. The reaction was quenched with about 2.5 mL of conc. NH₄OH and the resulting solid collected by filtration and washed with water. The solid was air dried to 740 mg and ˜2 g of silica gel was added. The mixture was dry-loaded on to a silica gel column and the product was eluted with a gradient of 10%/80%/10% EtOAe/hexane/DCM to 70%/20%/10% EtOAc/hexane/DCM, (40×150 mm col, 80 mL/min, 16+ minute run). The product containing fractions were combined and concentrated at reduced pressure to give the title compound. MS: m/z=315 (M+1).

### Intermediate 14

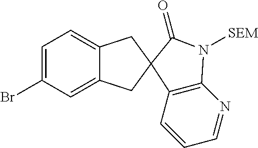

### (±)-5-Bromo-1′-{[2-trimethylsilyl)ethoxy]methyl}-1,3-dihydrospiro[indene-2,3′-pyrrolo[2,3-b]pyridin]-2-′(1′H)-one

To a solution of 1,2-bis(bromomethyl)-4-bromobenzene (40.9 g, 132 mmol) and 1-{[2-(trimethylsilyl)ethoxy]methyl}-1,3-dihydro-2H-pyrrolo[2,3-b]pyridin-2-one (31.5 g, 119 mmol, described in Intermediate 3) in MeOH (2 L) was added cesium carbonate (129 g, 397 mmol), portionwise, over 5 min. After 18 h, the mixture was concentrated to a volume of about 500 mL, then partitioned between EtOAc (1.5 L) and H₂O (1 L). The organic layer was washed with H₂O (1 L), then brine (500 mL), then dried over Na₂SO₄, filtered, and concentrated in vacuo. The crude product was purified by silica gel chromatography, eluting with a gradient of hexane:EtOAc from 100:0 to 0:100, to give the title compound. MS: m/z=445 (M+1).

### Intermediate 15

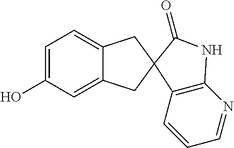

### (R)-5-Hydroxy-1,3-dihydrospiro[indene-2,3′-pyrrolo[2,3-b]pyridin]-2′(1′H)-one

A solution of sodium nitrite in water (275 mg, 3.98 mmole in 1.6 mL water) was slowly added to a cooled mixture of (R)-5-amino-1,3-dihydrospiro[indene-2,3′-pyrrolo[2,3-b]pyridin]-2′(1′H)-one (1 g, 3.98 mmol) (from Intermediate 6) in 8 mL of 10% H₂SO₄ (0.8 mL conc H₂SO₄+7.2 mL water) at 0° C. The ice bath was removed and the reaction allowed to stir at rt. The reaction was then placed into a 70° C. oil bath and heated to 100° C. Bubbling was observed and heating was continued until LCMS showed that the reaction was done. The reaction was slowly quenched/neutralized with ˜2 mL of conc. NH₄OH (pH is about 8 at the end of multiple sonications and beatings in order to get a stable pH and a solid suitable for filtration). The solid was collected by filtration and washed with water. The solid was then air dried and chromatographed by first mixing with ˜ twice the amount of silica and then dry loading on a silica gel column. The product was eluted with (10% MeOH/DCM). Concentration of the product containing fractions gave 445 mg of the product. MS: m/z=253 (M+1).

### Intermediate 16

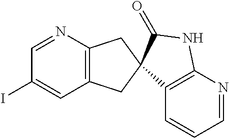

### (6S)-3-iodo-5,7-dihydrospiro cyclopenta[b]pyridine-6,3′-pyrrolo[2,3-b]pyridin]-2′(1′H)-one

A solution of sodium nitrite (0.562 g, 8.15 mmol) in water (2 mL) was slowly added to a solution of (6S)-3-amino-5,7-dihydrospiro[cyclopenta[b]pyridine-6,3′-pyrrolo[2,3-b]pyridin]-2′(1′H)-one, bis-hydrochloride salt (2.50 g, 7.69 mmol, described in Intermediate 8) in water (12 mL), THF (4 mL) and 37% HCl (1.9 mL) at 0° C. The reaction mixture was stirred at 0° C. for 90 min, poured onto saturated aqueous NaHCO₃ (100 mL) and extracted with 10% MeOH:CH₂Cl₂ (3×100 mL). The combined organic extracts were dried over Na₂SO₄, filtered, and concentrated in vacuo. The crude product was dried onto silica gel and purified by silica gel chromatography, eluting with a gradient of CH₂Cl₂:MeOH: NH₄OH from 100:0:0 to 90:10:1, to give the title compound. MS: m/z=364 (M+1).

### Intermediate 17

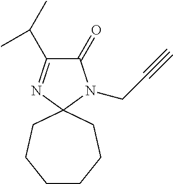

### 3-isopropyl-1-prop-2-yn-1-yl-1,4-diazaspiro[4.6]undec-3-en-2-one

A solution of 3-isopropyl-1,4-diazaspiro[4.6]undec-3-en-2-one (1.0 g, 4.80 mmol) in 8 mL DMF was added dropwise to a suspension of sodium hydride (0.230 g, 5.76 mmol) in DMF (5 ml) at 0° C. After the gas evolution had ceased, propargyl bromide (0.535 ml, 4.80 mmol) was added. After 15 minutes, the reaction was quenched, and partitioned between H₂O and ethyl acetate. The aqueous layer was extracted with ethyl acetate (2×30 mL), then the combined organic layers were washed with brine, dried over Na₂SO₄, filtered and concentrated in vacuo. The residue was purified on the Isco Rf system, with a biotage 100 g column, eluting with 0/100 to 30/70 ethyl acetate/hexanes gradient. The clean fractions were concentrated in vacuo to yield 3-isopropyl-1-prop-2-yn-1-yl-1,4-diazaspiro[4.6]undec-3-en-2-one. MS: m/z=247 (M+1).

### Intermediate 18

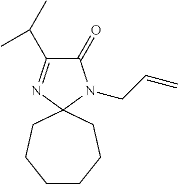

### 1-allyl-3-isopropyl-1,4-diazaspiro[4.6]undec-3-en-2-one

A solution of 3-isopropyl-1,4-diazaspiro[4.6]undec-3-en-2-one (1.0 g, 4.80 mmol) in 8 mL DMF dropwise was added to a suspension of 95% sodium hydride (0.218 g, 8.64 mmol) in DMF (5 ml) at 0° C. After the gas evolution had ceased, allyl bromide (0.515 ml, 5.95 mmol) in 1 mL of DMF was added. After stirring for 1 h, the reaction was quenched with H₂O at 0° C. The mixture was extracted with 3×30 mL of ethyl acetate. The combined organic layers were washed with brine, then dried over Na₂SO₄, filtered and concentrated in vacuo. The residue was purified on an Isco R^{f} system, using a 100 g biotage silica gel column, eluting with a gradient of 0/100 to 40/60 ethyl acetate/hexanes. The clean fractions were concentrated in vacuo to yield 1-allyl-3-isopropyl-1,4-diazaspiro[4.6]undec-3-en-2-one (0.93 g, 3.74 mmol, 78% yield). MS: m/z=249 (M+1).

It is understood that alternative methodologies may also be employed in the synthesis of these key intermediates. For instance, racemic reaction sequences may be utilized, followed by chiral separations at appropriate steps to provide compounds of the present invention. The exact choice of reagents, solvents, temperatures, and other reaction conditions, depends upon the nature of the intended product. In some cases, appropriate protecting group strategies may be used.

In some cases the final product may be further modified, for example, by manipulation of substituents. These manipulations may include, but are not limited to, reduction, oxidation, alkylation, acylation, and hydrolysis reactions which are commonly known to those skilled in the art.

In some cases the order of carrying out the foregoing reaction schemes may be varied to facilitate the reaction or to avoid unwanted reaction products. Additionally, various protecting group strategies may be employed to facilitate the reaction or to avoid unwanted reaction products. The following examples are provided so that the invention might be more fully understood. These examples are illustrative only and should not be construed as limiting the invention in any way.

### Example 1

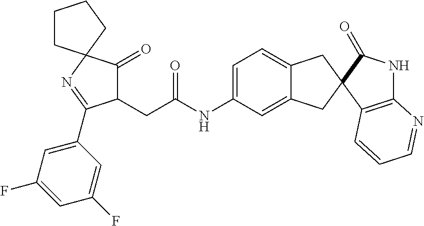

### 2-[2-(3,5-difluorophenyl)-4-oxo-1,3-diazaspiro[4.4]non-1-en-3-yl]-N-[(2R)-2′-oxo-1,1′,2′,3-tetrahydrospiro[indene-2,3′-pyrrolo[2,3-b]pyridin]-5-yl]acetamide

A mixture of [2-(3,5-difluorophenyl)-4-oxo-1,3-diazaspiro[4.4]non-1-en-3-yl]acetic acid (36.8 mg, 0.119 mmol, described in Intermediate 1), (R)-5-amino-1,3-dihydrospiro[indene-2,3′-pyrrolo[2,3-b]pyridin]-2′(UH)-one (30 mg, 0.119 mmol, described in Intermediate 4), and BOP reagent ((1H-1,2,3-benzotriazol-1-yloxy) [tris(dimethylamino)]phosphonium hexafluorophosphate, 52.8 mg, 0.179 mmol) and Hunigs base (31 mg, 44 uL, 0.24 mmol) in DMF (1 mL) was stirred at ambient temperature for 1 h. The reaction mixture was purified directly by HPLC using a reversed phase C18 column and eluting with a gradient of H₂O:CH₃CN:CF₃CO₂H from 90:10:0.1 to 5:95:0.1. The pure, product-containing fractions were concentrated in vacuo to give the title compound as the trifluoroacetate salt. MS: m/z=542 (M+1).

Essentially following the procedures outlined for Example 1, the compounds listed in Table 4 were prepared from intermediates 5, 7 and 8. The requisite amino carboxamides are described in the literature, or readily synthesized by one skilled in the art of organic synthesis. The carboxylic acids are commercially available, prepared by procedures substantially similar to those described for Intermediate 1, described in the literature, or readily synthesized by one skilled in the art of organic synthesis.

TABLE 4

| | | Mass |
|---|---|---|
| | | Spec |
| Example | Structure | (M + 1) |
| 2 | 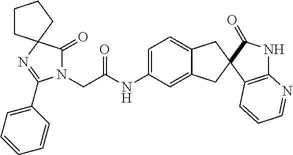 | 506 |
| 3 | 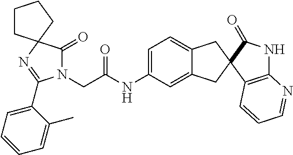 | 520 |
| 4 | 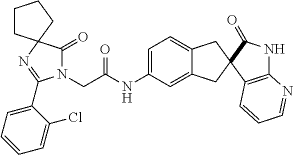 | 540 |
| 5 | 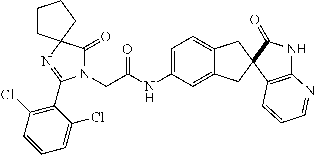 | 574 |
| 6 | 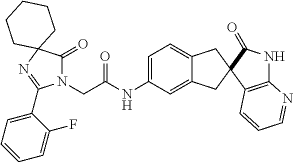 | 538 |
| 7 | 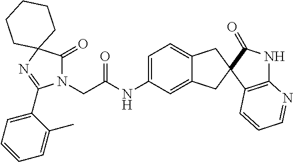 | 534 |
| 8 | 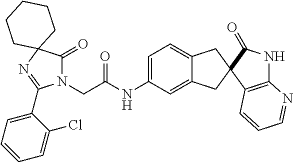 | 554 |
| 9 | 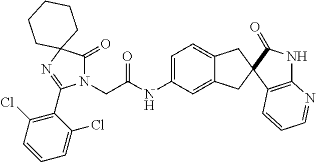 | 588 |
| 10 | 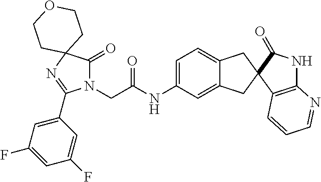 | 558 |
| 11 | 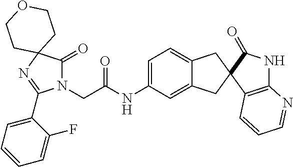 | 540 |
| 12 | 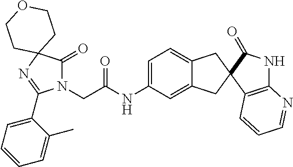 | 536 |
| 13 | 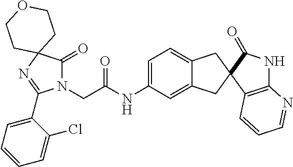 | 556 |
| 14 | 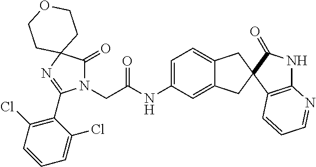 | 590 |
| 15 | 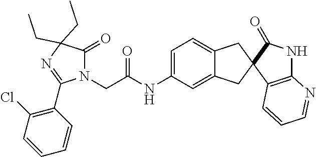 | 542 |
| 16 | 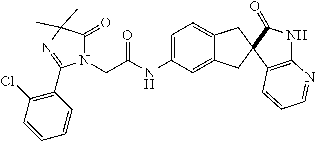 | 514 |
| 17 | 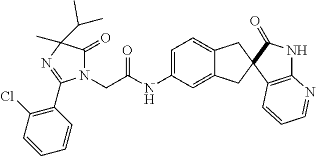 | 542 |
| 18 | 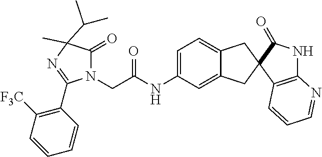 | 576 |
| 19 | 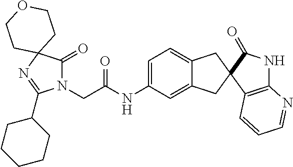 | 528 |
| 20 | 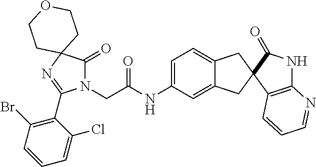 | 634 |
| 21 | 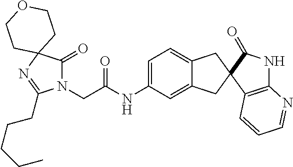 | 516 |
| 22 | 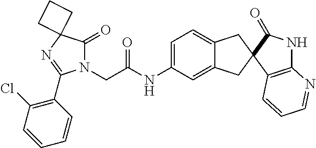 | 526 |
| 23 | 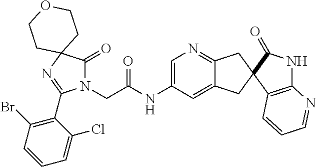 | 635 |
| 24 | 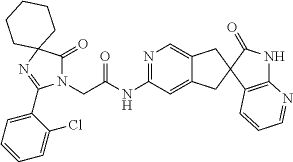 | 555 |
| 25 | 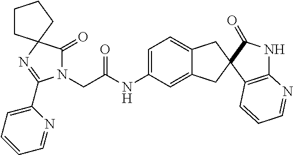 | 507 |
| 26 | 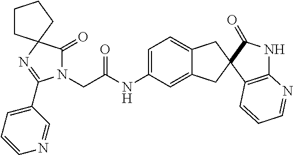 | 507 |
| 27 | 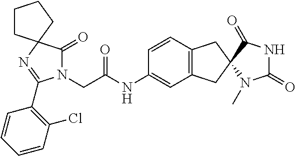 | 520.2 |
| 28 | 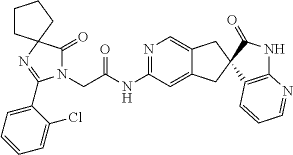 | 541.2 |
| 29 | 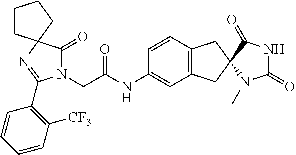 | 554.2 |
| 30 | 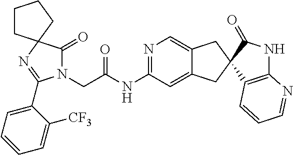 | 575.2 |
| 31 | 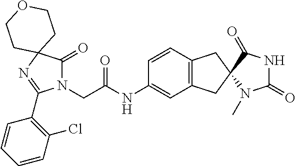 | 536.2 |
| 32 | 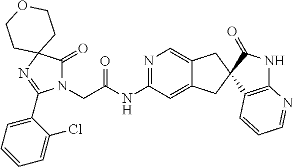 | 557.2 |
| 33 | 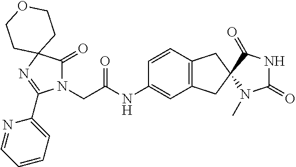 | 503.2 |
| 34 | 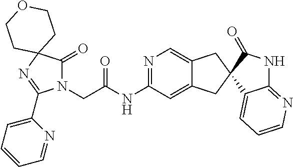 | 524.2 |
| 35 | 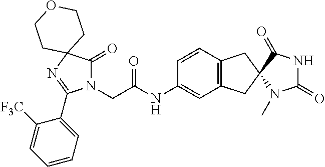 | 570.2 |
| 36 | 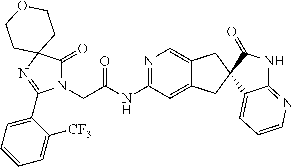 | 591.2 |
| 37 | 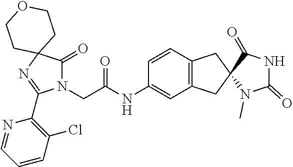 | 537.2 |
| 38 | 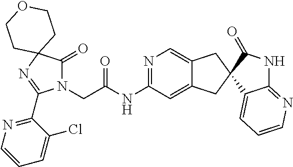 | 558.2 |

### Example 39

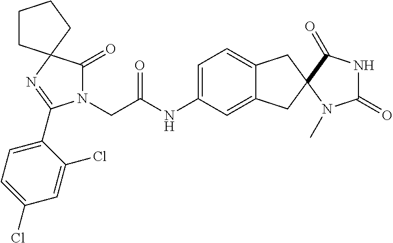

### 2-[2-(2,4-dichlorophenyl)-4-oxo-1,3-diazaspiro[4.4]non-1-en-3-yl]-N-[(4S)-3-methyl-2,5-dioxo-1′,3′-dihydrospiro[imidazolidine-4,2′-inden]-5′-yl]acetamide

The title compound was prepared from [2-(2,4-dichlorophenyl)-4-oxo-1,3-diazaspiro[4.4]non-1-en-3-yl]acetic acid (Intermediate 2,) and (4S)-5′-amino-3-methyl-1′,3′-dihydro-2H-5H-spiro[imidazolidine-4,2′-indene]-2,5-dione (Bell, I M., et al., PCT Int. Appl., WO 2004082605 A2 20040930) by a procedure substantially as described for Example 1. MS: m/z=554 (M+1).

Essentially following the procedures outlined for Example 1, the compounds listed in Table 5 were prepared from intermediates 5, 7, 8 and 12. The requisite amino carboxamides are described in the literature, or readily synthesized by one skilled in the art of organic synthesis. The carboxylic acids are prepared by procedures substantially similar to those described for Intermediates 12, described in the literature, or readily synthesized by one skilled in the art of organic synthesis.

TABLE 5

| Ex- | | MS |
|---|---|---|
| ample | Structure | (M + 1) |
| 40 | 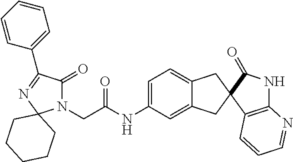 | 520 |
| 41 | 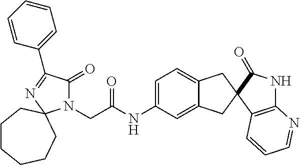 | 534 |
| 42 | 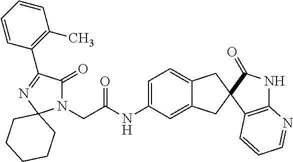 | 534 |
| 43 | 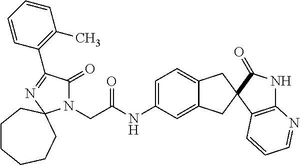 | 548 |
| 44 | 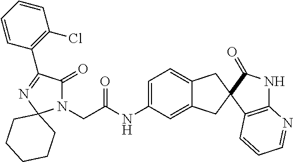 | 554 |
| 45 | 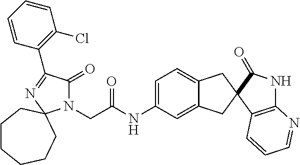 | 568 |
| 46 | 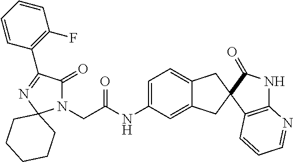 | 538 |
| 47 | 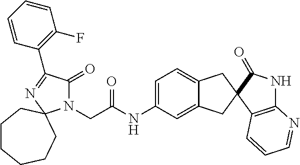 | 552 |
| 48 | 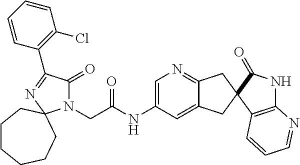 | 569 |
| 49 | 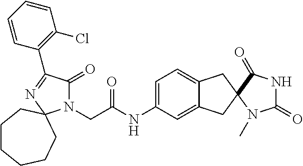 | 548 |
| 50 | 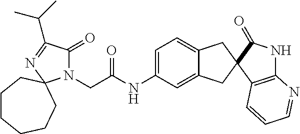 | 500 |
| 51 | 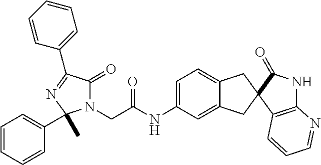 | 542 |
| 52 | 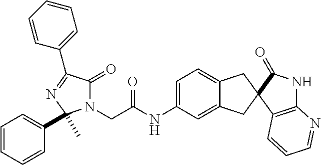 | 542 |
| 53 | 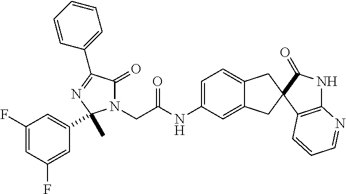 | 578 |
| 54 | 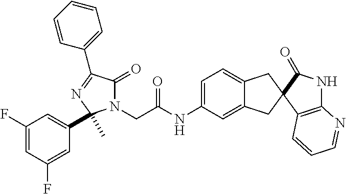 | 578 |
| 55 | 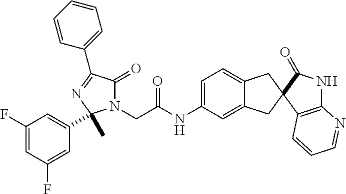 | 578 |
| 56 | 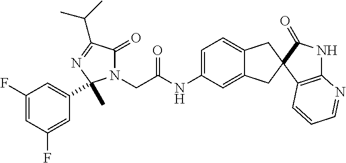 | 544 |
| 57 | 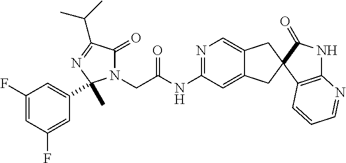 | 545 |
| 58 | 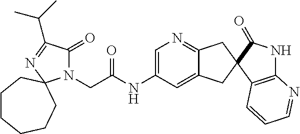 | 501 |
| 59 | 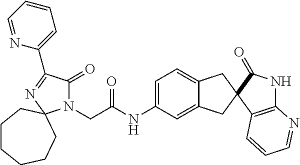 | 535 |
| 60 | 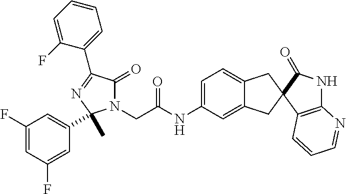 | 596 |
| 61 | 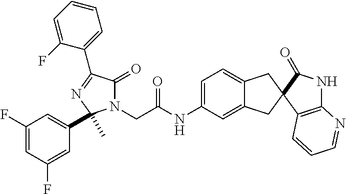 | 596 |
| 62 | 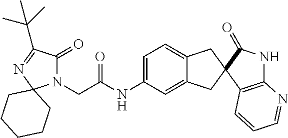 | 500 |
| 63 | 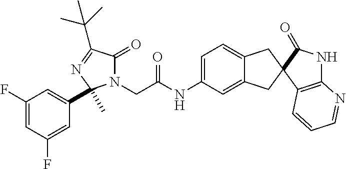 | 558 |
| 64 | 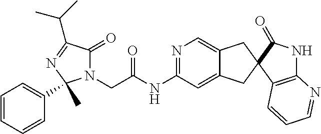 | 509 |
| 65 | 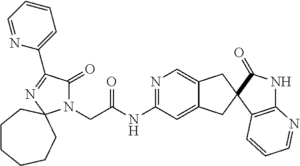 | 536 |
| 66 | 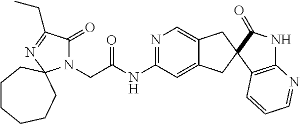 | 487 |
| 67 | 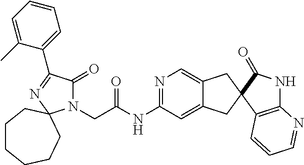 | 549 |
| 68 | 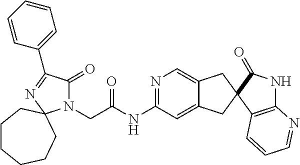 | 535 |
| 69 | 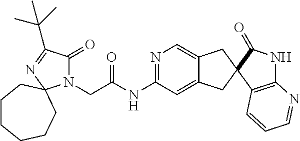 | 515 |
| 70 | 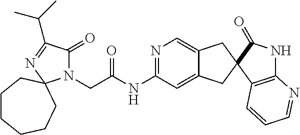 | 501 |
| 71 | 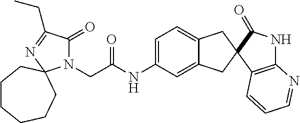 | 486 |
| 72 | 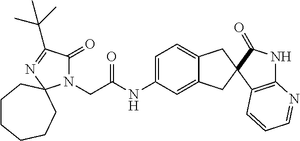 | 514 |
| 73 | 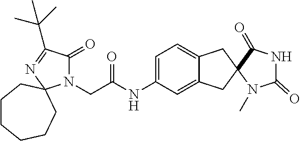 | 494 |
| 74 | 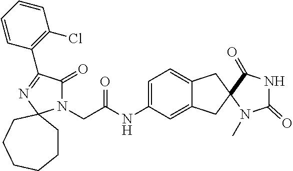 | 548 |

### Example 75

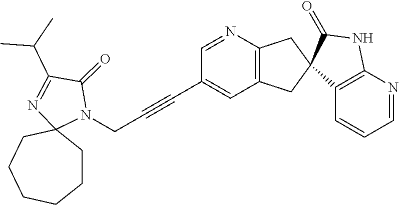

### (6S)-3-[3-(3-isopropyl-2-oxo-1,4-diazaspiro[4.6]undec-3-en-1-yl)prop-1-yn-1-yl]-5,7-dihydrospiro[cyclopenta[b]pyridine-6,3′-pyrrolo[2,3-b]pyridin]-2′(1′H)-one

To a degassed solution of 3-isopropyl-1-prop-2-yn-1-yl-1,4-diazaspiro[4.6]undec-3-en-2-one (102 mg, 0.413 mmol) and (6S)-3-iodo-5,7-dihydrospiro[cyclopenta[b]pyridine-6,3′-pyrrolo[2,3-b]pyridin)-2′(1′H)-one (150 mg, 0.413 mmol) in degassed DMF (4 mL) was added tetrakis(triphenylphosphine) palladium(0) (23.9 mg, 0.021 mmol), copper(I) iodide (7.9 mg, 0.041 mmol), and triethylamine (0.173 mL, 1.24 mmol). The reaction mixture was stirred at 50° C. for 18 h. Saturated aqueous NaHCO₃ (5 mL) was added and the quenched mixture was extracted with CH₂Cl₂ (2×30 mL). The combined organic extracts were dried over Na₂SO₄, filtered, and concentrated in vacuo. The crude product was purified by silica gel chromatography, eluting with a gradient of CH₂Cl₂:MeOH: NH₄OH from 100:0:0 to 95:5:1, to give the title compound. Treatment with HCl/ether provided the title compound as the HCl salt. MS: m/z=482 (M+1). HRMS: m/z=482.2770; calculated m/z=482.266 for C₂H₃₁N₅O₂.

### Example 76

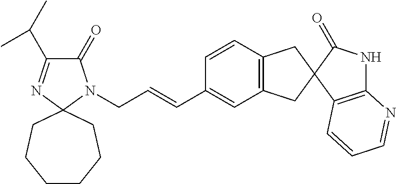

### 5-[(1E)-3-(3-isopropyl-2-oxo-1,4-diazaspiro[4.6]undec-3-en-1-yl)prop-1-en-1-yl]-1,3-dihydrospiro[indene-2,3′-pyrrolo[2,3-b]pyridin]-2′(1′H)-one

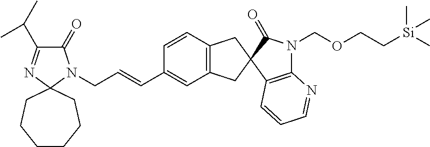

### Step A. 5-[(1E)-3-(3-isopropyl-2-oxo-1,4-diazaspiro[4.6]undec-3-en-1-yl)prop-1-en-1-yl]-1′-{[2-(trimethylsilyl)ethoxy]methyl}-1,3-dihydrospiro[indene-2,3′-pyrrolo[2,3-b]pyridin]-2′(1′H)-one

1-allyl-3-isopropyl-1,4-diazaspiro[4.6]undec-3-en-2-one (300 mg, 1.208 mmol), (2R)-5-bromo-1′-{[2-(trimethylsilyl)ethoxy]methyl}-1,3-dihydrospiro[indene-2,3′-pyrrolo[2,3-b]pyridin]-2′(1′H)-one (646 mg, 1.449 mmol), PdOAc₂ (81 mg, 0.362 mmol), potassium carbonate (167 mg, 1.208 mmol), and tri-tert-butylphosphine (0.149 mL, 0.604 mmol) were suspended in degassed (N₂) DMF (3 mL). The suspension was degassed for 10 min more, then microwaved at 150° C. for 30 min. The reaction was diluted with ethyl acetate, and then filtered through a plug of celite. The filtrate was washed with 3×30 mL ice water, then brine. The organic layer was dried over Na₂SO₄, filtered and concentrated in vacuo. The residue was purified on the Isco R^{f} system, using an Isco 80 g column, eluting with 40/60 to 100/0 ethyl acetate/hexanes. The clean fractions were concentrated in vacuo to yield 5-[(1E)-3-(3-isopropyl-2-oxo-1,4-diazaspiro[4.6]undec-3-en-1-yl)prop-1-en-1-yl]-1′-{[2-(trimethylsilyl)ethoxy]methyl}-1,3-dihydrospiro[indene-2,3′-pyrrolo[2,3-b]pyridin]-2′(1′H)-one (0.224 g, 0.365 mmol, 30.3% yield). MS: 771/z=249 (M+1).

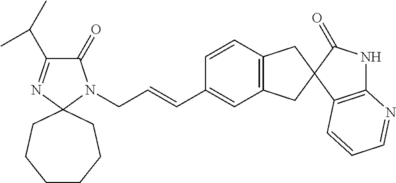

### Step B. 5-[(1E)-3-(3-isopropyl-2-oxo-1,4-diazaspiro[4.6]undec-3-en-1-yl)prop-1-en-1-yl]-1,3-dihydrospiro[indene-2,3′-pyrrolo[2,3-b]pyridin]-2′(1′H)-one

5-[(1E)-3-(3-isopropyl-2-oxo-1,4-diazaspiro[4.6]undec-3-en-1-yl)prop-1-en-1-yl]-1′-{[2-(trimethylsilyl)ethoxy])methyl}-1,3-dihydrospiro[indene-2,3′-pyrrolo[2,3-b]pyridin]-2′(1′H)-one (740 mg, 1.207 mmol) was dissolved in dichloromethane (5 mL) and TFA (3 mL, 38.9 mmol). After 1 h, the reaction was concentrated in vacuo. The residue was dissolved in methanol (5 mL), then 1M sodium hydroxide_{(aq)} (2 mL, 2.000 mmol) and ethylenediamine (0.5 mL, 7.40 mmol) were added. After 2 h, the reaction was concentrated in vacuo. The residue was partitioned between ethyl acetate and H₂O. The aqueous layer was washed with 2×30 mL of ethyl acetate. The combined organic layers were washed with brine, then dried over sodium sulfate and concentrated in vacuo. The residue was purified on an Isco Rf, eluting with 30/70 to 70/30 ethyl acetate/hexanes on a 50 g biotage column. The clean fractions were concentrated in vacuo to yield 5-[(1E)-3-(3-isopropyl-2-oxo-1,4-diazaspiro[4.6]undec-3-en-1-yl)prop-1-en-1-yl]-1,3-dihydrospiro[indene-2,3′-pyrrolo[2,3-b]pyridin]-2′(1′H)-one MS: m/z=483 (M+1). HRMS: m/z=483.2773 (M+1); calculated m/z=for C₃₁H₃₄N₄O₂.

Essentially following the procedures outlined for Examples 54 and 55, the compounds listed in Table 6 were prepared from intermediates prepared by procedures similar to those described above. The requisite intermediates are described in the literature, or readily synthesized by one skilled in the art of organic synthesis. The allyl and propargyl substituted coupling partners are prepared by procedures substantially similar to those described for Intermediates 17 and 18, and readily synthesized by one skilled in the art of organic synthesis.

TABLE 6

| Ex- | | MS |
|---|---|---|
| ample | Structure | (M +1) |
| 77 | 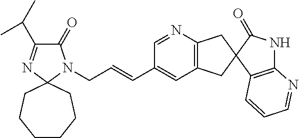 | 484 |
| 78 | 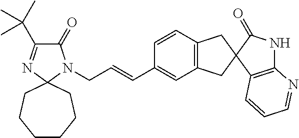 | 497 |
| 79 | 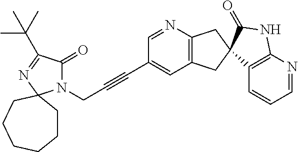 | 496 |
| 80 | 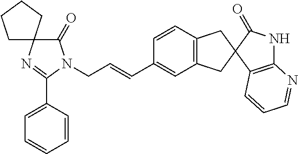 | 489 |
| 81 | 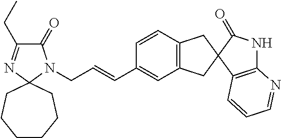 | 469 |
| 82 | 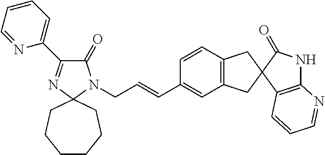 | 518 |
| 83 | 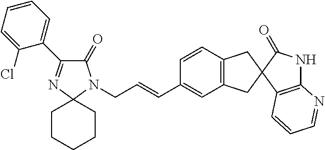 | 537 |
| 84 | 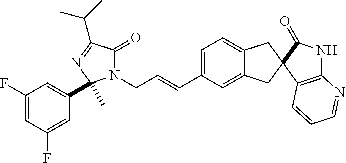 | 527 |
| 85 | 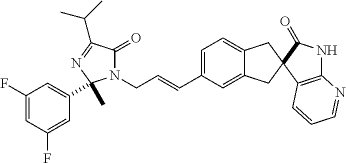 | 527 |
| 86 | 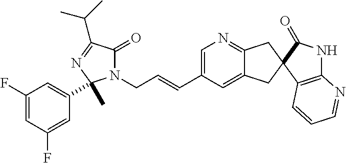 | 528 |
| 87 | 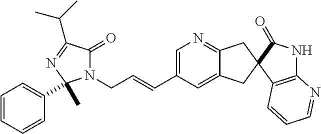 | 492 |
| 88 | 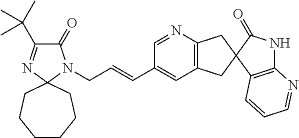 | 498 |
| 89 | 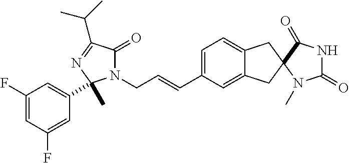 | 507 |
| 90 | 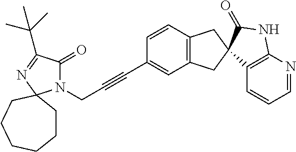 | 481 |
| 91 | 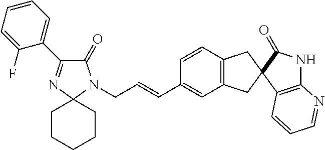 | 521 |
| 92 | 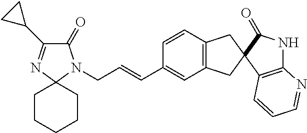 | 481 |
| 93 | 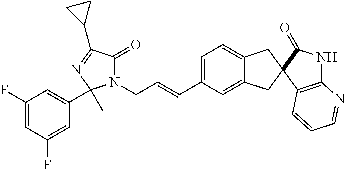 | 525 |
| 94 | 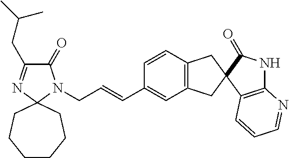 | 497 |
| 95 | 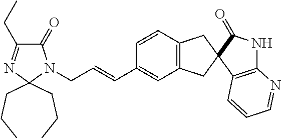 | 469 |
| 96 | 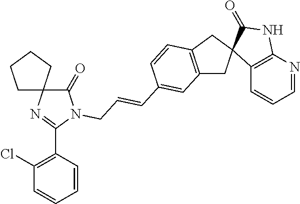 | 524 |
| 97 | 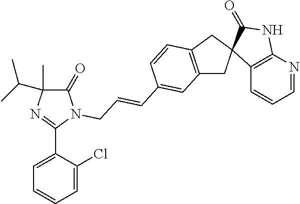 | 526 |
| 98 | 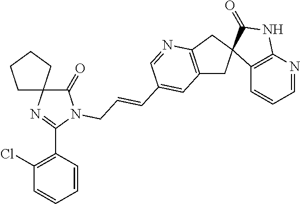 | 525 |
| 99 | 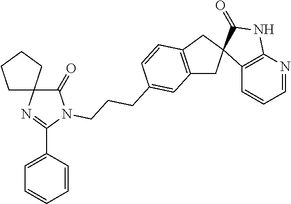 | 491 |
| 100 | 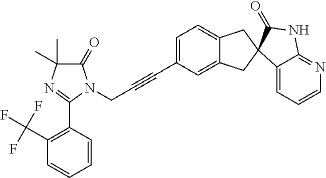 | 529 |
| 101 | 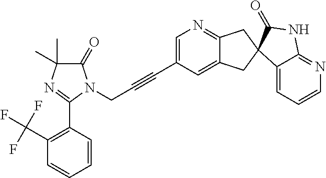 | 530 |
| 102 | 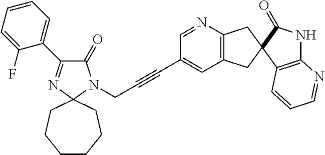 | 534 |
| 103 | 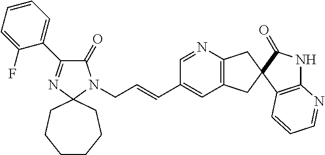 | 536 |
| 104 | 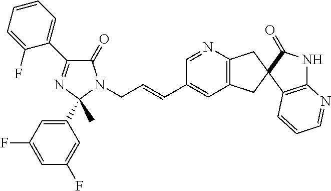 | 580 |
| 105 | 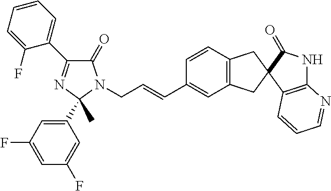 | 579 |
| 106 | 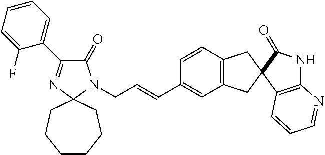 | 535 |
| 107 | 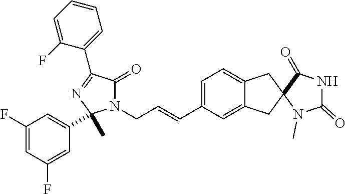 | 559 |
| 108 | 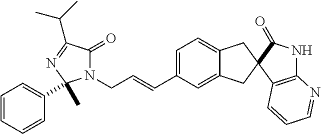 | 491 |
| 109 | 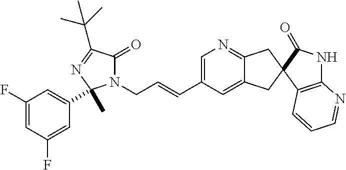 | 542 |
| 110 | 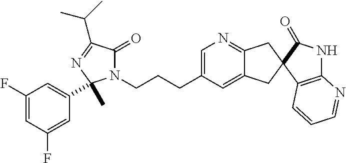 | 530 |
| 111 | 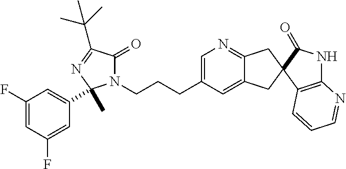 | 544 |

Although specific enantiomers and diastereomers appear in the above Examples and Intermediates, it is well understood by those skilled in the art that modifications to reaction conditions and reagents (for example, but not limited to: using the opposite chirality for starting materials; different catalysts; using the opposite chirality for reagents; choosing to use a different enantiomer or diastereomer subsequent to a chiral resolution) will provide alternative enantiomers and diastereomers, all of which are included in the spirit and scope of the invention. It is intended that all of the possible optical isomers and diastereomers in mixtures and as pure or partially purified compounds are included within the ambit of this invention. The present invention is meant to comprehend all such isomeric forms of these compounds.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A compound of the formula I: 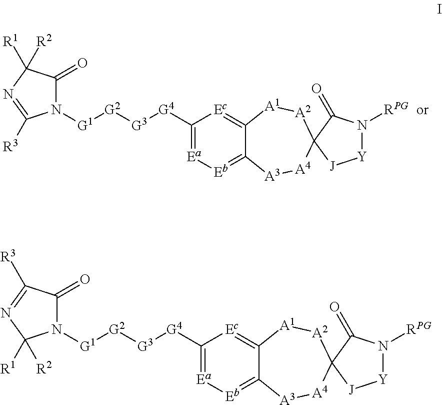
wherein:
A¹ and A³ are independently selected from:
(1) —O—,
(2) —S(O)ᵥ—,
(3) —Si(OR^{a})(C₁₋₄alkyl)-, where alkyl is unsubstituted or substituted with 1-5 halo,
(4) —Si(C₁₋₄alkyl)₂-, where each alkyl is independently unsubstituted or substituted with 1-5 halo,
(5) —CR^{e}R^{f}—,
(6) —N(R⁴)—,
(7) —(C═O)—, and
(8) a bond,
A² and A4 are independently selected from:
(1) —O—,
(2) —S(O)ᵥ—,
(3) —Si(OR^{a})(C₁₋₄alkyl)-, where alkyl is unsubstituted or substituted with 1-5 halo,
(4) —Si(C₁₋₄alkyl)₂-, where each alkyl is independently unsubstituted or substituted with 1-5 halo,
(5) —CR^{e}R^{f}—,
(6) —N(R⁴)—, and
(7) —(C═O)—,
E^{a} is selected from:
(1) —C(R^{5a})═,
(2) —N═, and
(3) —(N⁺—O⁻)═;
E^{b} is selected from:
(1) —C(R^{5b})═;
(2) —N═, and
(3) —(N⁺—O⁻)═;
E^{c} is selected from:
(1) —C(R^{5c})═,
(2) —N═, and
(3) —(N⁺—O⁻)═;
G¹ is selected from:
(1) a bond,
(2) —CR^{e}R^{f}—,
(3) —CR^{e}R^{f}—CH₂—,
(4) —CH₂—CR^{e}R^{f}—, and
(5) —(C═O)═;
G² is selected from:
(1) a bond,
(2) —CR^{e}R^{f}—,
(3) —CR^{e}R^{f}—CH₂—,
(4) —CH₂—CR^{e}R^{f}
(5) —(C═O)—,
(6) —N(R⁴)—,
(7) —O—,
(8) —S(O)ᵥ—,
(9) —SiR^{g}R^{h}—,
(10) —C(Rⁱ)═C(R^{j})—, and
(11) —C≡C—;
G³ is selected from:
(1) a bond,
(2) —CR^{e}R^{f}—,
(3) —N(R⁴)—,
(4) —O—,
(5) —S(O)ᵥ—,
(6) —SiR^{g}R^{h}—,
(7) —C(Rⁱ)═C(R^{j})—,
(8) —C≡C—, and
(9) —(C═O)—;
G⁴ is selected from:
(1) —CR^{e}R^{f}—,
(2) —N(R⁴)—,
(3) —O—,
(4) —S(O)ᵥ—,
(5) —SiR^{g}R^{h}—,
(6) —C(Rⁱ)═C(R^{j})—, and
(7) —C≡C—;
R¹ and R² are each independently selected from:
(1) hydrogen,
(2) —C₁₋₆alkyl, which is unsubstituted or substituted with 1-5 substituents each independently selected from:
(a) halo,
(b) —OR^{a},
(c) —C₃₋₆cycloalkyl,
(d) phenyl or heterocycle, wherein said heterocycle is selected from: pyridyl, pyrimidinyl, pyrazinyl, pyridazinyl, piperidinyl, piperazinyl, pyrrolidinyl, thienyl, morpholinyl, thiazolyl, indolyl, indazolyl, benzimidazolyl, and oxazolyl, which phenyl or heterocycle is unsubstituted or substituted with 1-5 substituents each independently selected from:
(i) halo,
(ii) —C₁₋₆alkyl, which is unsubstituted or substituted with 1-5 halo,
(iii) —OR^{a},
(iv) —NR^{b}R^{c},
(v) —CN, and
(vi) oxo;
(e) —CO₂R^{a},
(f) —C(═O)NR^{b}R^{c},
(g) —S(O)ᵥR^{d},
(h) —CN,
(i) —NR^{b}R^{c},
(j) —N(R^{b})C(═O)R^{a},
(k) —N(R^{b})SO₂R^{d},
(l)—CF₃,
(m)—O—CO₂R^{d},
(n) —O—(C═O)—NR^{b}R^{c},
(o)—NR^{b}—(C═O)—NR^{b}R^{c}, and
(p) —C(═O)R^{a},
(3) —C₃₋₈cycloalkyl, which is unsubstituted or substituted with 1-5 substituents each independently selected from:
(a) halo,
(b) —CN,
(c) —C₁₋₄alkyl, which is unsubstituted or substituted with 1-3 halo, and
(d) —OR^{a},
(4) phenyl or heterocycle, wherein said heterocycle is selected from: pyridyl, pyrimidinyl, pyrazinyl, pyridazinyl, piperidinyl, piperazinyl, pyrrolidinyl, thienyl, morpholinyl, thiazolyl and oxazolyl, which phenyl or heterocycle is unsubstituted or substituted with 1-5 substituents each independently selected from:
(a) halo,
(b) —OR^{a},
(c) —C₃₋₆cycloalkyl,
(d) phenyl, which is unsubstituted or substituted with 1-5 substituents each independently selected from:
(i) halo,
(ii) —C₁₋₆alkyl, which is unsubstituted or substituted with 1-6 halo, and
(iii) —OR^{a},
(e) —CO₂R^{a},
(f) —C(═O)NR^{b}R^{c},
(g) —S(O)ᵥR^{d},
(h) —CN,
(i) —NR^{b}R^{c},
(j) —N(R^{b})C(═O)R^{a},
(k) —N(R^{b})SO₂R^{d},
(l) —O—CO₂R^{d},
(m) —O—(C═O)—NR^{b}R^{c},
(n) —NR^{b}—(C═O)—NR^{b}R^{c},
(o) —C(═O)R^{a},
(p) —C₁₋₆alkyl, which is unsubstituted or substituted with 1-6 halo, and
(q) oxo;
(5) halo,
(6) —OR^{a},
(7) —CN,
(8) —CO₂R^{a},
(9) —N(R^{b})C(═O)R^{a},
(10) —NR^{b}R^{c},
(11) —C(═O)NR^{b}R^{c}, and
(12) —O(C═O)R^{a};
or R¹ and R² and the carbon atom or atoms to which they are attached join to form a ring selected from cyclopropyl, cyclobutyl, cyclopentyl, cyclohexyl, cycloheptyl, cyclooctyl, cyclononyl, cyclobutenyl, cyclopentenyl, cyclohexenyl, cycloheptenyl, cyclooctenyl, dioxolanyl, dioxanyl, aziridinyl, azetidinyl, pyrrolidinyl, piperidinyl, piperazinyl, morpholinyl, tetrahydrofuranyl, tetrahydropyranyl, tetrahydrothiapyranyl, oxetanyl, thietanyl and tetrahydrothienyl, and benzofused analogs of any of the aforementioned rings, wherein any sulfur atoms present are optionally oxidized to the sulfone or sulfoxide, and said ring is unsubstituted or substituted with 1-6 substituents each independently selected from:
(a) —C₁₋₆alkyl, which is unsubstituted or substituted with 1-3 substituents each independently selected from:
(i) halo,
(ii) —OR^{a},
(iii) —C₃₋₆cycloalkyl,
(iv) —CO₂R^{a},
(v) —NR^{b}R^{c},
(vi) —S(O)ᵥR^{d},
(vii) —C(═O)NR^{b}R^{c}, and
(viii) phenyl,
(b) —C₃₋₆cycloalkyl, wherein the C₃₋₆cycloalkyl group is optionally fused to the ring, and which C₃₋₆cycloalkyl group is unsubstituted or substituted with 1-3 substituents each independently selected from:
(i) halo,
(ii) —OR^{a},
(iii) —C₃₋₆cycloalkyl,
(iv) —CO₂R^{a},
(v) —NR^{b}R^{c},
(vi) —S(O)ᵥR^{d},
(vii) —C(═O)NR^{b}R^{c}, and
(viii) phenyl,
(c) phenyl or heterocycle, wherein heterocycle is selected from: pyridyl, pyrimidinyl, pyrazinyl, pyridazinyl, piperidinyl, piperazinyl, pyrrolidinyl, thienyl, morpholinyl, imidazolyl, (uranyl, tetrahydrofuranyl, thiazolyl and oxazolyl, wherein the phenyl or heterocycle is optionally fused to the ring, and which phenyl or heterocycle is unsubstituted or substituted with 1-5 substituents each independently selected from:
(i) halo,
(ii) —C₁₋₆alkyl, which is unsubstituted or substituted with 1-5 halo,
(iii) —OR^{a},
(iv) —CO₂R^{a},
(v) —O(C═O)R^{a},
(vi) —CN,
(vii) —NR^{b}R^{c},
(viii) oxo,
(ix) —C(═O)NR^{b}R^{c},
(x) —N(R^{b})C(═O)R^{a},
(xi) —N(R^{b})CO₂R^{a},
(xii) —O(C═O)NR^{b}R^{c}, and
(xiii) —S(O)ᵥR^{d},
(d) —OR^{a},
(e) —CO₂R^{a},
(f) —C(═O)NR^{b}R^{c},
(g) —S(O)ᵥR^{d},
(h) —CN,
(i) halo,
(j) —NR^{b}R^{c},
(k) —N(R^{b})C(═O)R^{a},
(l) —N(R^{b})SO₂R^{d},
(m) —O—CO₂R^{d},
(n) —O—(C═O)—NR^{b}R^{c},
(o)—NR^{b}—(C═O)—NR^{b}R^{c},
(p) —C(═O)R^{a}, and
(q) oxo;
R³ is selected from the group consisting of:
(1) —C₁₋₁₀alkyl, which is unsubstituted or substituted with 1-5 substituents each independently selected from:
(a) halo,
(b) —OR^{a},
(c) —C₃₋₆cycloalkyl,
(d) phenyl or heterocycle, wherein said heterocycle is selected from: pyridyl, pyrimidinyl, pyrazinyl, pyridazinyl, piperidinyl, piperazinyl, pyrrolidinyl, thienyl, morpholinyl, thiazolyl, indolyl, indazolyl, benzimidazolyl, and oxazolyl, which phenyl or heterocycle is unsubstituted or substituted with 1-5 substituents each independently selected from:
(i) halo,
(ii) —C₁₋₆alkyl, which is unsubstituted or substituted with 1-5 halo,
(iii) —OR^{a},
(iv) —NR^{b}R^{c},
(v) —CN, and
(vi) oxo;
(e) —CO₂R^{a},
(f) —C(═O)NR^{b}R^{c},
(g) —S(O)ᵥR^{d},
(h) —CN,
(i) —NR^{b}R^{c},
(j) —N(R^{b})C(═O)R^{a},
(k) —N(R^{b})SO₂R^{d},
(l) —CF₃,
(m) —O—CO₂R^{d},
(n) —O—(C═O)—NR^{b}R^{c},
(p) —NR^{b}—(C═O)—NR^{b}R^{c}, and
(q) —C(═O)R^{a},
(2) —C₃₋₁₀cycloalkyl or benzofused —C₃₋₁₀cycloalkyl, which is unsubstituted or substituted with 1-5 substituents each independently selected from:
(a) halo,
(b) —CN,
(c) —C₁₋₄alkyl, which is unsubstituted or substituted with 1-3 halo, and
(d) —OR^{a}, and
(3) phenyl, naphthyl, tetrahydronaphthyl, indanyl, biphenyl, phenanthryl, anthryl, azepinyl, azepanyl, azetidinyl, benzimidazolyl, benzisoxazolyl, benzofuranyl, benzofurazanyl, benzopyranyl, benzothiopyranyl, benzofuryl, 1,3-benzodioxolyl, benzothiazolyl, benzothienyl, benzoxazolyl, benzopyrazolyl, benzotriazolyl, chromanyl, cinnolinyl, dibenzofuranyl, dihydrobenzofuryl, dihydrobenzothienyl, dihydrobenzothiopyranyl, dihydrobenzothiopyranyl sulfone, furyl, furanyl, imidazolidinyl, imidazolinyl, imidazolyl, indolinyl, indolyl, isochromanyl, isoindolinyl, isoquinolinyl, isothiazolidinyl, isothiazolyl, morpholinyl, naphthyridinyl, oxazolyl, oxadiazolyl, 2-oxoazepinyl, 4-oxonaphthyridinyl, 2-oxopiperazinyl, 2-oxopiperidinyl, 2-oxopyrrolidinyl, 2-oxopyridyl, 2-oxoquinolinyl, piperidyl, piperazinyl, pyrazinyl, pyrazolidinyl, pyrazolyl, pyridazinyl, pyridinyl, pyridyl, pyrimidinyl, pyrimidyl, pyrrolidinyl, pyrrolyl, quinazolinyl, quinolinyl, quinoxalinyl, tetrahydrofuranyl, tetrahydrofuryl, tetrahydroimidazopyridinyl, tetrahydroisoquinolinyl, tetrahydroquinolinyl, tetrazolyl, thiamorpholinyl, thiamorpholinyl sulfoxide, thiamorpholinyl sulfone, thiazolyl, thiazolinyl, thienofuryl, thienothienyl, thienyl, triazolyl, isoxazolyl, tetrahydrothienyl, tetrahydropyranyl, oxetanyl, tetrahydrothiapyranyl, and thietanyl, each of which is unsubstituted or substituted with 1-5 substituents each independently selected from R⁶;
R⁴ is independently selected from:
(1) hydrogen,
(2) —C(═O)R^{a},
(3) —CO₂R^{a},
(4) —S(═O)R^{d},
(5) —SO₂R^{d},
(6) —C(═O)NR^{b}R^{c},
(7) —C₁₋₆alkyl, which is unsubstituted or substituted with 1-5 substituents each independently selected from:
(a) halo,
(b) —OR^{a},
(e) —C₃₋₆cycloalkyl,
(d) phenyl or heterocycle, wherein said heterocycle is selected from: pyridyl, pyrimidinyl, pyrazinyl, pyridazinyl, piperidinyl, piperazinyl, pyrrolidinyl, thienyl, morpholinyl, thiazolyl and oxazolyl, which phenyl or heterocycle is unsubstituted or substituted with 1-5 substituents each independently selected from:
(i) halo,
(ii) —C₁₋₆alkyl, which is unsubstituted or substituted with 1-5 halo, and
(iii) —OR^{a},
(iv) —NR^{b}R^{c},
(v) —C(═O)R^{a},
(vi) —CO₂R^{a}, and
(vii) oxo,
(e) —CO₂R^{a},
(f) —C(═O)NR^{b}R^{c},
(g) —S(O)ᵥR^{d},
(h) —CN,
(i) —NR^{b}R^{c},
(j) —N(R^{b})C(═O)R^{a},
(k) —N(R^{b})SO₂R^{d},
(l) —CF₃,
(m) —O—CO₂R^{d},
(n) —O—(C═O)—NR^{b}R^{c},
(o) —NR^{b}—(C═O)—NR^{b}R^{c}, and
(p) —C(═O)R^{a},
(8) —C₃₋₆cycloalkyl, which is unsubstituted or substituted with 1-6 substituents each independently selected from:
(a) halo,
(b) —CN,
(c) —OR^{a}, and
(d) C₁₋₆alkyl, which is unsubstituted or substituted with 1-6 halo;
R^{5a}, R^{5b} and R^{5c} are each independently selected from:
(1) hydrogen,
(2) —C₁₋₆alkyl, which is unsubstituted or substituted with 1-6 halo,
(3) halo,
(4) —OR^{a}, and
(5) —CN;
R⁶ is selected from:
(1) —C₁₋₆alkyl, which is unsubstituted or substituted with 1-5 substituents each independently selected from:
(a) halo,
(b) —OR^{a},
(c) —C₃₋₆cycloalkyl,
(d) phenyl or heterocycle, wherein said heterocycle is selected from: pyridyl, pyrimidinyl, pyrazinyl, pyridazinyl, piperidinyl, piperazinyl, pyrrolidinyl, thienyl, morpholinyl, thiazolyl and oxazolyl, which phenyl or heterocycle is unsubstituted or substituted with 1-5 substituents each independently selected from:
(i) halo,
(ii) —C₁₋₆alkyl, which is unsubstituted or substituted with 1-5 halo, and
(iii) —OR^{a},
(e) —CO₂R^{a},
(f) —C(═O)NR^{b}R^{c},
(g) —S(O)ᵥR^{d},
(h) —CN,
(i) —NR^{b}R^{c},
(j) —N(R^{b})C(═O)R^{a},
(k) —N(R^{b})SO₂R^{d},
(l) —CF₃,
(m) —O—CO₂R^{d},
(n) —O—(C═O)—NR^{b}R^{c},
(o) —NR^{b}—(C═O)—NR^{b}R^{c}, and
(p) —C(═O)R^{a},
(2) —C₃₋₆cycloalkyl, which is unsubstituted or substituted with 1-5 substituents each independently selected from:
(a) halo,
(b) —CN,
(c) which is unsubstituted or substituted with 1-5 halo,
(d) —OR^{a}, and
(e) phenyl, which is unsubstituted or substituted with 1-5 substituents where the substituents are each independently selected from:
(i) —OR^{a},
(ii) halo,
(iii) —CN, and
(iv) —C₁₋₆alkyl, which is unsubstituted or substituted with 1-5 halo,
(3) phenyl or heterocycle, wherein said heterocycle is selected from: pyridyl, pyrimidinyl, pyrazinyl, pyridazinyl, piperidinyl, piperazinyl, pyrrolidinyl, morpholinyl, thiazolyl and oxazolyl, which phenyl or heterocycle is unsubstituted or substituted with 1-5 substituents each independently selected from:
(a) halo,
(h) —OR^{a},
(c) —C₃₋₆cycloalkyl,
(d) phenyl, which is unsubstituted or substituted with 1-5 substituents each independently selected from:
(i) halo,
(ii) which is unsubstituted or substituted with 1-6 halo, and
(iii) —OR^{a},
(e) —CO₂R^{a},
(f) —C(═O)NR^{b}R^{c},
(g) —S(O)ᵥR^{d},
(h) —CN,
(i) —NR^{b}R^{c},
(j) —N(R^{b})C(═O)R^{a},
(k) —N(R^{b})SO₂R^{d},
(l) —O—CO₂R^{d},
(m) —O—(C═O)—NR^{b}R^{c},
(n) —NR^{b}—(C═O)—NR^{b}R^{c},
(o) —C(═O)R^{a}, and
(p) —C₁₋₆alkyl, which is unsubstituted or substituted with 1-6 halo,
(4) halo,
(5) oxo,
(6) —OR^{a},
(7) —CN,
(8) —CO₂R^{a},
(9) —C(═O)R^{a},
(10) —NR^{b}R^{c},
(11) —S(O)ᵥR^{d},
(12) —C(═O)NR^{b}R^{c},
(13) —O—CO₂R^{d},
(14) —N(R^{b})CO₂R^{d},
(15) —O—(C═O)—NR^{b}R^{c},
(16) —NR^{b}—(C═O)—NR^{b}R^{c},
(17) —SO₂NR^{b}R^{c},
(18) —N(R^{b})SO₂R^{d},
and two R⁶ groups on the same or adjacent atoms together with the atom(s) to which they are attached may join to form a ring selected from cyclopropyl, cyclobutyl, cyclopentyl, cyclohexyl, cycloheptyl, aziridinyl, azetidinyl, pyrrolidinyl, piperidinyl, piperazinyl, morpholinyl, thietanyl and tetrahydrothienyl, wherein the sulfur is optionally oxidized to the sulfone or sulfoxide, which ring is unsubstituted or substituted with 1-5 substituents each independently selected from:
(a) —C₁₋₆alkyl, which is unsubstituted or substituted with 1-3 substituents each independently selected from:
(i) halo,
(ii) —OR^{a},
(iii) —C₃₋₆cycloalkyl,
(iv) —CO₂R^{a},
(v) —NR^{b}R^{c},
(vi) —S(O)ᵥR^{d},
(vii) —C(═O)NR^{b}R^{c}, and
(viii) phenyl,
(b) phenyl or heterocycle, wherein said heterocycle is selected from: pyridyl, pyrimidinyl, pyrazinyl, pyridazinyl, piperidinyl, piperazinyl, pyrrolidinyl, thienyl, morpholinyl, thiazolyl and oxazolyl, which phenyl or heterocycle is unsubstituted or substituted with 1-5 substituents each independently selected from:
(i) halo,
(ii) —C₁₋₆alkyl, which is unsubstituted or substituted with 1-5 halo, and
(iii) —OR^{a},
(c) —OR^{a},
(d) halo,
(e) —CO₂R^{a},
(f) —C(═O)NR^{b}R^{c},
(g) —S(O)ᵥR^{d},
(h) —CN,
(i) —NR^{b}R^{c},
(j) —N(R^{b})C(═O)R^{a},
(k) —N(R^{b})SO₂R^{d},
(l) —O—CO₂R^{d},
(m)—O—(C═O)—NR^{b}R^{c},
(n) —NR^{b}—(C═O)—NR^{b}R^{c}, and
(o)—C(═O)R^{a};
R^{PG} is selected from:
(1) hydrogen,
(2) —C₁₋₆alkyl which is unsubstituted or substituted with 1-5 halo,
(3) —CH₂OR^{a},
(4) —CH₂—O—CH₂CH₂Si(CH₃)₃,
(5) —CH₂OP(═O)(OR^{c})₂,
(6) —(CH₂)ₖ-phenyl, which is unsubstituted or substituted with 1-3 substituents each independently selected from:
(a) halo,
(b) —OR^{a},
(c) —CN, and
(d) which is unsubstituted or substituted with 1-6 halo;
J is selected from:
(1) ═C(R^{7a})—,
(2) —CR⁸R⁹—,
(3) —C(═O)—, and
(4) —N(R^{b})—;
Y is selected from:
(1) ═C(R^{7b})—,
(2) —CR⁸R⁹—,
(3) —C(═O)—,
(4) ═N—, and
(5) —N(R^{b})—;
R^{7a} and R^{7b} are each independently selected from:
(1) hydrogen,
(2) which is unsubstituted or substituted with 1-5 substituents each independently selected from:
(a) halo,
(b) —OR^{a},
(c) —C₃₋₆cycloalkyl,
(d) phenyl or heterocycle, wherein said heterocycle is selected from: imidazolyl, oxazolyl, pyridyl, pyrimidinyl, pyrazinyl, pyridazinyl, piperidinyl, piperazinyl, pyrrolidinyl, thiazolyl, thienyl, triazolyl, isoxazolyl and morpholinyl, which phenyl or heterocycle is unsubstituted or substituted with 1-3 substituents each independently selected from:
(i) halo,
(ii) —OR^{a},
(iii) —CN, and
(iv) C₁₋₆alkyl, which is unsubstituted or substituted with 1-6 halo,
(3) phenyl or heterocycle, wherein heterocycle is selected from: imidazolyl, oxazolyl, pyridyl, pyrimidinyl, pyrazinyl, pyridazinyl, tetrahydrofuryl, piperidinyl, piperazinyl, pyrrolidinyl, azetidinyl, thiazolyl, thienyl, triazolyl, isoxazolyl and morpholinyl, which phenyl or heterocycle is unsubstituted or substituted with 1-3 substituents each independently selected from:
(a) halo,
(b) —OR^{a},
(c) —C₃₋₆cycloalkyl,
(d) —C₁₋₄alkyl which is unsubstituted or substituted with 1-6 halo, and
(e) phenyl, which is unsubstituted or substituted with 1-5 substituents each independently selected from:
(i) halo,
(ii) —C₁₋₆alkyl, which is unsubstituted or substituted with 1-6 halo, and
(iii) —OR^{a},
(4) halo,
(5) —OR^{a},
(6) —CN,
(7) —CO₂R^{a},
(8) —NR^{b}R^{c}, and
(9) —C(═O)NR^{b}R^{c};
or R^{7a} and R^{7b} and the atom(s) to which they are attached join to form a ring selected from cyclopentenyl, cyclohexenyl, phenyl, pyridyl, pyrimidinyl, pyrazinyl, pyridazinyl, furanyl, dihydrofuranyl, dihydropyranyl, thiazolyl, isothiazolyl, oxazolyl, isoxazolyl, imidazolyl, triazolyl, thienyl, dihydrothienyl and dihydrothiopyranyl, which ring is unsubstituted or substituted with 1-5 substituents each independently selected from:
(a) which is unsubstituted or substituted with 1-3 substituents each independently selected from:
(i) halo,
(ii) —OR^{a},
(iii) —C₃₋₆cycloalkyl,
(iv) phenyl or heterocycle, wherein heterocycle is selected from pyridyl, pyrimidinyl, pyrazinyl, pyridazinyl, piperidinyl, piperazinyl, pyrrolidinyl, thienyl and morpholinyl, which phenyl or heterocycle is unsubstituted or substituted with 1-5 substituents each independently selected from:
(I) —OR^{a},
(II) halo,
(III) —CN, and
(IV) —C₁₋₆alkyl which is unsubstituted or substituted with 1-6 halo,
(v) —CO₂R^{a},
(vi) —NR^{b}R^{c},
(vii) —S(O)ᵥR^{d},
(viii) —C(═O)NR^{b}R^{c},
(ix) —N(R^{b})CO₂R^{a}, and
(x) —N(R^{b})SO₂R^{d},
(b) phenyl or heterocycle, wherein said heterocycle is selected from pyridyl, pyrimidinyl, pyrazinyl, pyridazinyl, piperidinyl, azetidinyl, piperazinyl, pyrrolidinyl, thienyl and morpholinyl, which phenyl or heterocycle is unsubstituted or substituted with 1-5 substituents each independently selected from:
(i) halo,
(ii) —OR^{a},
(iii) —CN, and
(iv) —C₁₋₆alkyl which is unsubstituted or substituted with 1-6 halo,
(c) halo,
(d) —S(O)ᵥR^{d},
(e) —OR^{a},
(f) —CN,
(g) —C(═O)R^{a},
(h) —NR^{b}R^{c},
(i) —C(═O)NR^{b}R^{o},
(l) —CO₂R^{a},
(k) —(NR^{b})CO₂R^{a},
(l) —O—(C═O)—NR^{b}R^{c},
(m) —(NR^{b})—(C═O)—NR^{b}R^{c},
(n) oxido,
(o) oxo, and
(p)—(NR^{b})SO₂R^{d};
R⁸ and R⁹ are each independently selected from:
(1) hydrogen,
(2) halo,
(3) —OR^{a},
(4) —C₁₋₆alkyl, which is unsubstituted or substituted with 1-4 substituents each independently selected from:
(a) halo,
(b) —OR^{a},
(c) —CN,
(d) phenyl or heterocycle, wherein said heterocycle is selected from pyridyl, pyrimidinyl, thienyl, pyridazinyl, piperidinyl, azetidinyl, piperazinyl, pyrrolidinyl, morpholinyl, tetrahydrofuranyl, tetrahydropyranyl and pyrazinyl, which phenyl or heterocycle is unsubstituted or substituted with 1-5 substituents each independently selected from:
(i) —OR^{a},
(ii) halo,
(iii) —CN,
(iv) —C₁₋₆alkyl which is unsubstituted or substituted with 1-6 halo,
(5) phenyl or heterocycle wherein heterocycle is selected from pyridyl, pyrimidinyl, thienyl, pyridazinyl, piperidinyl, azetidinyl, piperazinyl, pyrrolidinyl, morpholinyl, tetrahydrofuranyl, tetrahydropyranyl and pyrazinyl, which phenyl or heterocycle is unsubstituted or substituted with 1-5 substituents each independently selected from:
(a) halo,
(b) —CN,
(c) —OR^{a},
(d) nitro,
(e) —C₁₋₆alkyl which is unsubstituted or substituted with 1-6 halo;
and R⁸ and R⁹ and the atom to which they are attached may join to form a 4-, 5-, or 6-membered ring optionally containing a heteroatom selected from N, O, and S, wherein the sulfur is optionally oxidized to the sulfone or sulfoxide, which ring is unsubstituted or substituted with 1-4 substituents each independently selected from:
(a) halo,
(b) —OR^{a},
(c) —C₁₋₆alkyl, which is unsubstituted or substituted with 1-6 halo, and
(d) phenyl;
R^{a} is independently selected from:
(1) hydrogen,
(2) C₁₋₆alkyl, which is unsubstituted or substituted with 1-7 substituents each independently selected from:
(a) halo,
(b) —O—C₁₋₆alkyl, which is unsubstituted or substituted with 1-6 halo,
(c) hydroxyl,
(d) —CN, and
(e) phenyl or heterocycle wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:
(i) halo,
(ii) —O—C₁₋₆alkyl, which is unsubstituted or substituted with 1-6 halo,
(iii) —CN,
(iv) nitro,
(v) hydroxyl, and
(vi) —C₁₋₆alkyl, which is unsubstituted or substituted with 1-6 halo,
(3) phenyl or heterocycle wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:
(a) halo,
(b) —CN,
(c) —O—C₁₋₆alkyl, which is unsubstituted or substituted with 1-6 halo,
(d) nitro,
(e) hydroxyl, and
(f) —C₁₋₆alkyl, which is unsubstituted or substituted with 1-6 halo, and
(4) —C₃₋₆cycloalkyl, which is unsubstituted or substituted with 1-6 halo;
R^{b} and R^{e} are independently selected from:
(1) hydrogen,
(2) C₁₋₆alkyl, which is unsubstituted or substituted with 1-7 substituents each independently selected from:
(a) halo,
(b) —OR^{a},
(c) —CN,
(d) —CO₂R^{a},
(e) phenyl or heterocycle, wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:
(i) halo,
(ii) —OR^{a},
(iii) —C₁₋₆alkyl, which is unsubstituted or substituted with 1-6 halo, and
(iv) nitro,
(3) phenyl or heterocycle, wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:
(a) halo,
(b) —OR^{a},
(c) —C₁₋₆alkyl, which is unsubstituted or substituted with 1-6 halo,
(d) —C₃₋₆cycloalkyl, which is unsubstituted or substituted with 1-6 halo,
(e) —CN, and
(f) —CO₂R^{a},
(4) —C₃₋₆cycloalkyl, which is unsubstituted or substituted with 1-6 halo;
and R^{b} and R^{c} and the nitrogen to which they are attached may join to form a 4-, 5-, or 6-membered ring optionally containing an additional heteroatom selected from N, O, and S, wherein the sulfur is optionally oxidized to the sulfone or sulfoxide, which ring is unsubstituted or substituted with 1-4 substituents each independently selected from:
(a) halo,
(b) —OR^{a}, and
(c) —C₁₋₆alkyl, which is unsubstituted or substituted with 1-6 halo, and
(d) phenyl;
R^{d} is independently selected from:
(1) C₁₋₆alkyl, which is unsubstituted or substituted with 1-4 substituents each independently selected from:
(a) halo,
(b) —OR^{a},
(c) —CO₂R^{a},
(d) —CN, and
(e) phenyl or heterocycle, wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:
(i) halo,
(ii) —OR^{a},
(iii) —C₁₋₆alkyl, which is unsubstituted or substituted with 1-6 halo, and
(iv) nitro,
(2) phenyl or heterocycle, wherein said heterocycle is selected from pyridyl, pyrimidinyl, thienyl, pyridazinyl, piperidinyl, azetidinyl, furanyl, piperazinyl, pyrrolidinyl, morpholinyl, tetrahydrofuranyl, tetrahydropyranyl and pyrazinyl, which phenyl or heterocycle is unsubstituted or substituted with 1-3 substituents each independently selected from:
(a) halo,
(b) —OR^{a},
(c) —C₁₋₆alkyl, which is unsubstituted or substituted with 1-6 halo,
(d) —C₃₋₆cycloalkyl, which is unsubstituted or substituted with 1-6 halo
(e) —CN, and
(f) —CO₂R^{a}, and
(3) —C₃₋₆cycloalkyl, which is unsubstituted or substituted with 1-6 halo;
R^{e} and R^{f} are independently selected from:
(1) hydrogen,
(2) —C₁₋₄alkyl, which is unsubstituted or substituted with 1-6 halo,
(3) —OR^{a},
(4) —CN,
(5) halo,
(6) phenyl, and
(7) benzyl;
and R^{e} and R^{f} and the carbon atom or atoms to which they are attached may join to form a 3-, 4-, 5-, or 6-membered ring optionally containing a heteroatom selected from N, O, and S, wherein the sulfur is optionally oxidized to the sulfone or sulfoxide, which ring is unsubstituted or substituted with 1-4 substituents each independently selected from:
(a) halo,
(b) —OR^{a},
(c) —C₁₋₆alkyl, which is unsubstituted or substituted with 1-6 halo, and
(d) phenyl;
R^{g} and R^{h} are independently selected from:
(1) —C₁₋₄alkyl, which is unsubstituted or substituted with 1-6 halo,
(2) —OR^{a},
(3) —C₃₋₆cycloalkyl, which is unsubstituted or substituted with 1-6 halo,
(4) phenyl, and
(5) benzyl;
and R^{g} and R^{h} and the silicon atom to which they are attached may join to form a 3-, 4-, 5-, or 6-membered ring optionally containing a heteroatom selected from N, O, and S, wherein the sulfur is optionally oxidized to the sulfone or sulfoxide, which ring is unsubstituted or substituted with 1-4 substituents each independently selected from:
(a) halo,
(b) —OR^{a},
(c) which is unsubstituted or substituted with 1-3 halo, and
(d) phenyl;
Rⁱ and R^{j} are independently selected from:
(1) hydrogen,
(2) —C₁₋₄alkyl, which is unsubstituted or substituted with 1-6 halo,
(3) —OR^{a},
(4) halo,
(5) phenyl, and
(6) benzyl;
v is 0, 1, or 2;
k is 0, 1, or 2;
and pharmaceutically acceptable salts thereof.

2. The compound of claim 1 having the formula Ia: 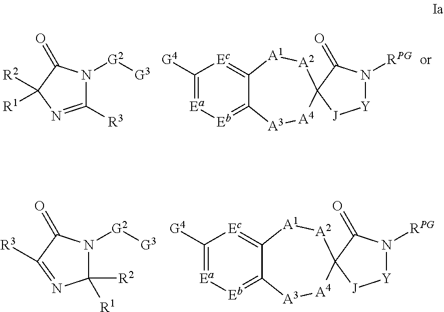
and pharmaceutically acceptable salts thereof.

3. The compound of claim 1 having the formula Ib: 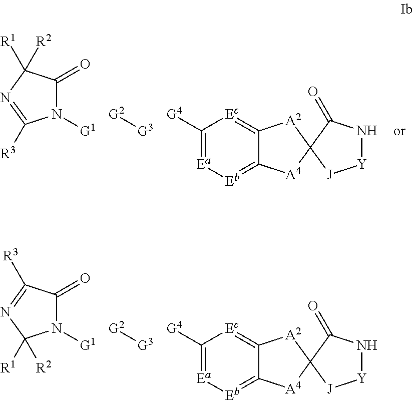
and pharmaceutically acceptable salts thereof.

4. The compound of claim 1 having the formula Ic: 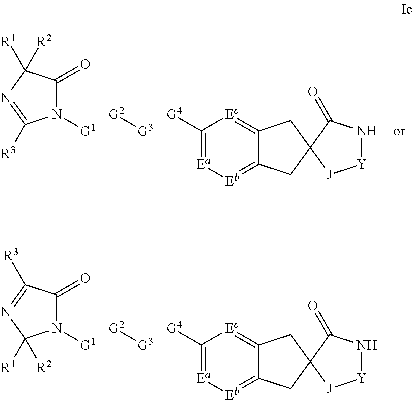
and pharmaceutically acceptable salts thereof.

5. The compound of claim 1 having the formula Id: 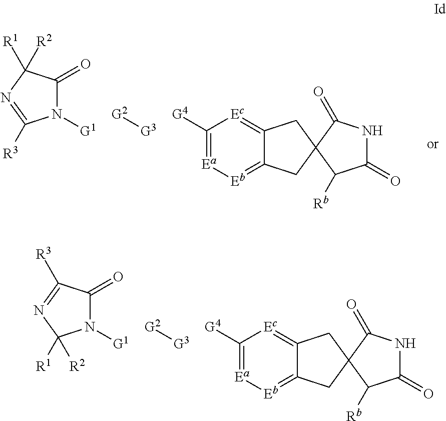
and pharmaceutically acceptable salts thereof.

6. The compound of claim 1 having the formula Ie: 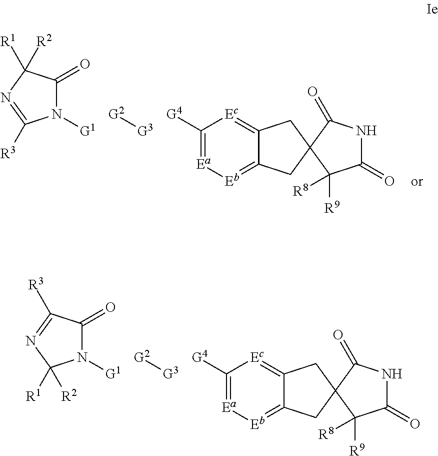
and pharmaceutically acceptable salts thereof.

7. The compound of claim 1 having the formula If: 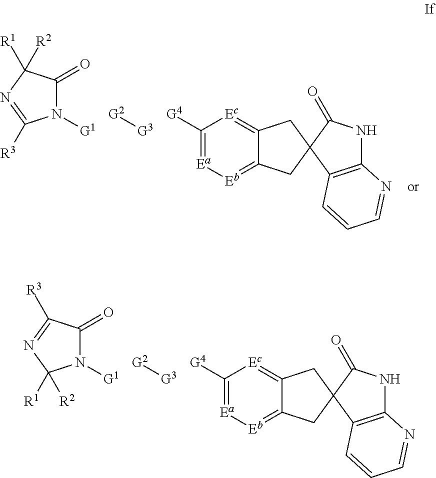
and pharmaceutically acceptable salts thereof.

8. The compound of claim 1, wherein R^{5a}, R^{5b} and R^{5c} are independently selected from hydrogen, halo, and —C₁₋₆alkyl, which is unsubstituted or substituted with 1-5 fluoro.

9. The compound of claim 1, wherein R¹ and R² are independently selected from
(1) hydrogen,
(2) —C₁₋₆alkyl, which is unsubstituted or substituted with 1-5 substituents where the substitutents are each independently selected from: halo, phenyl, and —OR^{a},
(3) —C₃₋₆cycloalkyl, which is unsubstituted or substituted with 1-5 fluoro,
(4) phenyl or heterocycle, which is unsubstituted or substituted with 1-5 halo,
(5) halo,
(6) —OR^{a},
(7) —NR^{b}R^{c}, and
(8) —O(C═O)R^{a}.

10. The compound of claim 1, wherein R¹ and R² and the carbon atom or atoms to which they are attached join to form a ring selected from cyclopropyl, cyclobutyl, cyclopentyl, cyclohexyl, cycloheptyl, cyclooctyl, cyclopentenyl, cyclohexenyl, cycloheptenyl, dioxolanyl, dioxanyl, tetrahydrofuranyl, tetrahydropyranyl, pyrrolidinyl, and piperidinyl, which ring is unsubstituted or substituted with 1-6 substituents each independently selected from:
(1) —C₁₋₆alkyl, which is unsubstituted or substituted with 1-3 substituents where the substitutents are each independently selected from: halo, —OR^{a}, and phenyl,
(2) —C₃₋₆cycloalkyl, wherein the C₃₋₆cycloalkyl group is optionally fused to the ring, and which C₃₋₆cycloalkyl group is unsubstituted or substituted with 1-3 substituents each independently selected from: halo, —OR^{a}, and phenyl,
(3) phenyl or heterocycle, wherein heterocycle is selected from: pyridyl, pyrimidinyl, pyrazinyl, pyridazinyl, piperidinyl, piperazinyl, pyrrolidinyl, thienyl, morpholinyl, thiazolyl and oxazolyl, wherein the phenyl or heterocycle is optionally fused to the ring, and which phenyl or heterocycle is unsubstituted or substituted with 1-3 substituents each independently selected from: halo, —OR^{a}, and —C₁₋₄alkyl, which is unsubstituted or substituted with 1-5 fluoro,
(4) halo,
(5) oxo,
(6) —CO₂R^{a}, and
(7) —C(═O)R^{a}.

11. The compound of claim 1, wherein R⁴ is selected from: hydrogen, —C(═O)R^{a}, —CO₂R^{a}, —SO₂R^{d}, and —C₁₋₆alkyl, which is unsubstituted or substituted with 1-5 fluoro.

12. The compound of claim 1, wherein J is ═C(R^{7a})—, —CR⁸R⁹— or N(R^{b})—,

13. The compound of claim 1, wherein Y is ═C(R^{7b})—, —CR⁸R⁹— or —C(═O)—.

14. The compound of claim 1, wherein R^{7a} and R^{7b} are independently selected from:
(1) hydrogen,
(2) —C₁₋₄alkyl, which is unsubstituted or substituted with 1-3 substituents each independently selected from: halo, —OR^{a}, —C₃₋₆cycloalkyl, and phenyl,
(3) phenyl or heterocycle, wherein heterocycle is selected from: pyridyl, pyrimidinyl, pyrazinyl, thiazolyl, thienyl, triazolyl, isoxazolyl and morpholinyl, which phenyl or heterocycle is unsubstituted or substituted with 1-3 substituents each independently selected from: —C₁₋₄alkyl which is unsubstituted or substituted with 1-3 halo, —OR^{a}, and halo,
(4) halo,
(5) OR^{a}, and
(6) —NR^{b}R^{c}.

15. The compound of claim 1, wherein R^{7a} and R^{7b} and the atom(s) to which they are attached join to form a ring selected from cyclohexenyl, phenyl, pyridyl, pyrimidinyl, pyrazinyl, pyridazinyl, furanyl, oxazolyl, isoxazolyl, imidazolyl, and thienyl, which ring is unsubstituted or substituted with 1-3 substituents each independently selected from:
(1) —C₁₋₆alkyl, which is unsubstituted or substituted with 1-3 substituents each independently selected from: halo, OR^{a}, —CO₂R^{a}, —NR^{b}R^{c}, and CONR^{b}R^{c},
(2) phenyl or heterocycle, wherein heterocycle is selected from pyridyl, pyrimidinyl, pyrazinyl, pyridazinyl, piperidinyl, azetidinyl, piperazinyl, pyrrolidinyl, thienyl and morpholinyl, which phenyl or heterocycle is unsubstituted or substituted with 1-3 substituents each independently selected from: halo, OR^{a} and —C₁₋₄alkyl, which is unsubstituted or substituted with 1-3 fluoro,
(3) halo,
(4) OR^{a},
(5) —CN,
(6) —NR^{b}R^{c},
(7) CONR^{b}R^{c}, and
(8) oxo.

16. The compound of claim 1 having the Formula Ig: 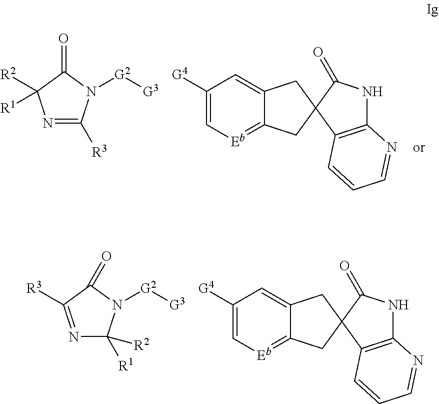
wherein:
R¹ and R² are independently selected from:
(1) —C₁₋₆alkyl, which is unsubstituted or substituted with 1-5 substituents each independently selected from:
(a) halo,
(b) —OR^{a},
(c) —C₃₋₆cycloalkyl,
(d) phenyl or heterocycle, wherein said heterocycle is selected from: pyridyl, pyrimidinyl, pyrazinyl, pyridazinyl, piperidinyl, piperazinyl, pyrrolidinyl, thienyl, morpholinyl, thiazolyl, indolyl, indazolyl, benzimidazolyl, and oxazolyl, which phenyl or heterocycle is unsubstituted or substituted with 1-5 substituents each independently selected from:
(i) halo,
(ii) —C₁₋₆alkyl, which is unsubstituted or substituted with 1-5 halo,
(iii) —OR^{a},
(iv) —NR^{b}R^{c},
(v) —CN, and
(vi) oxo;
(e) —CO₂R^{a},
(f) —C(═O)NR^{b}R^{c},
(g) —S(O)ᵥR^{d},
(h) —CN,
(i) —NR^{b}R^{c},
(j) —N(R^{b})C(═O)R^{a},
(k)—N(R^{b})SO₂R^{d},
(l) —CF₃,
(m)—O—CO₂R^{d},
(n) —O—(C═O)—NR^{b}R^{c},
(o)—NR^{b}—(C═O)—NR^{b}R^{c}, and
(p) —C(═O)R^{a}, and
(2) phenyl or heterocycle, wherein said heterocycle is selected from: pyridyl, pyrimidinyl, pyrazinyl, pyridazinyl, piperidinyl, piperazinyl, pyrrolidinyl, thienyl, morpholinyl, thiazolyl and oxazolyl, which phenyl or heterocycle is unsubstituted or substituted with 1-5 halo;
or R¹ and R² and the carbon atom or atoms to which they are attached join to form a ring selected from cyclopropyl, cyclobutyl, cyclopentyl, cyclohexyl, cycloheptyl, cyclooctyl, cyclononyl, cyclobutenyl, cyclopentenyl, cyclohexenyl, cycloheptenyl, cyclooctenyl, dioxolanyl, dioxanyl, aziridinyl, azetidinyl, pyrrolidinyl, piperidinyl, piperazinyl, morpholinyl, tetrahydrofuranyl, tetrahydropyranyl, tetrahydrothiapyranyl, oxetanyl, thietanyl and tetrahydrothienyl, wherein the sulfur is optionally oxidized to the sulfone or sulfoxide, which ring is unsubstituted or substituted with 1-6 substituents each independently selected from:
(a) —C₁₋₆alkyl, which is unsubstituted or substituted with 1-3 substituents each independently selected from:
(i) halo,
(ii) —OR^{a},
(iii) —C₃₋₆cycloalkyl,
(iv) —CO₂R^{a},
(v) —NR^{b}R^{c},
(vi) —S(O)ᵥR^{d},
(vii) —C(═O)NR^{b}R^{c}, and
(viii) phenyl,
(b) —C₃₋₆cycloalkyl, wherein the C₃₋₆cycloalkyl group is optionally fused to the ring, and which C₃₋₆cycloalkyl group is unsubstituted or substituted with 1-3 substituents each independently selected from:
(i) halo,
(ii) —OR^{a},
(iii) —C₃₋₆cycloalkyl,
(iv) —CO₂R^{a},
(v) —NR^{b}R^{c},
(vi) —S(O)ᵥR^{d},
(vii) —C(═O)NR^{b}R^{c}, and
(viii) phenyl,
(c) phenyl or heterocycle, wherein heterocycle is selected from: pyridyl, pyrimidinyl, pyrazinyl, pyridazinyl, piperidinyl, piperazinyl, pyrrolidinyl, thienyl, morpholinyl, imidazolyl, furanyl, tetrahydrofuranyl, thiazolyl and oxazolyl, wherein the phenyl or heterocycle is optionally fused to the ring, and which phenyl or heterocycle is unsubstituted or substituted with 1-5 substituents each independently selected from:
(i) halo,
(ii) —C₁₋₆alkyl, which is unsubstituted or substituted with 1-5 halo,
(iii) —OR^{a},
(iv) —CO₂R^{a},
(v) —O(C═O)R^{a},
(vi) —CN,
(vii) —NR^{b}R^{c},
(viii) oxo,
(ix) —C(═O)NR^{b}R^{c},
(x) —N(R^{b})C(═O)R^{a},
(xi) —N(R^{b})CO₂R^{a},
(xii) —O(C═O)NR^{b}R^{c}, and
(xiii) —S(O)ᵥR^{d},
(d) —OR^{a},
(e) —CO₂R^{a},
(f) —C(═O)NR^{b}R^{c},
(g) —S(O)ᵥR^{d},
(h) —CN,
(i) halo,
(j) —NR^{b}R^{c},
(k) —N(R^{b})C(═O)R^{a},
(l) —N(R^{b})SO₂R^{d},
(m) —O—CO₂R^{d},
(n) —O—(C═O)—NR^{b}R^{c},
(o) —NR^{b}—(C═O)—NR^{b}R^{c},
(q) —C(═O)R^{a}, and
(r) oxo;
R³ is selected from:
(1) —C₃₋₁₀cycloalkyl or benzofused —C₃₋₁₀cycloalkyl, which is unsubstituted or substituted with 1-5 halo, and
(2) phenyl, naphthyl, tetrahydronaphthyl, indanyl, biphenyl, phenanthryl, anthryl, azepinyl, azepanyl, azetidinyl, benzimidazolyl, benzisoxazolyl, benzofuranyl, benzofurazanyl, benzopyranyl, benzothiopyranyl, benzofuryl, 1,3-benzodioxolyl, benzothiazolyl, benzothienyl, benzoxazolyl, benzopyrazolyl, benzothiazolyl, chromanyl, cinnolinyl, dibenzofuranyl, dihydrobenzofuryl, dihydrobenzothienyl, dihydrobenzothiopyranyl, dihydrobenzothiopyranyl sulfone, furyl, furanyl, imidazolidinyl, imidazolinyl, imidazolyl, indolinyl, indolyl, isochromanyl, isoindolinyl, isoquinolinyl, isothiazolidinyl, isothiazolyl, morpholinyl, naphthyridinyl, oxazolyl, oxadiazolyl, 2-oxoazepinyl, 4-oxonaphthyridinyl, 2-oxopiperazinyl, 2-oxopiperidinyl, 2-oxopyrrolidinyl, 2-oxopyridyl, 2-oxoquinolinyl, piperidyl, piperazinyl, pyrazinyl, pyrazolidinyl, pyrazolyl, pyridazinyl, pyridinyl, pyridyl, pyrimidinyl, pyrimidyl, pyrrolidinyl, pyrrolyl, quinazolinyl, quinolinyl, quinoxalinyl, tetrahydrofuranyl, tetrahydrofuryl, tetrahydroimidazopyridinyl, tetrahydroisoquinolinyl, tetrahydroquinolinyl, tetrazolyl, thiamorpholinyl, thiamorpholinyl sulfoxide, thiamorpholinyl sulfone, thiazolyl, thiazolinyl, thienofuryl, thienothienyl, thienyl, triazolyl, isoxazolyl, tetrahydrothienyl, tetrahydropyranyl, oxetanyl, tetrahydrothiapyranyl, and thietanyl, each of which is unsubstituted or substituted with 1-5 substituents each independently selected from R⁶;
-G2-G3-G⁴- is selected from the group consisting of:
—CH₂—C(O)—NH—
—CH₂—CH₂—CH₂—,
—CH₂—CH═CH—,
—CH₂—C≡C— and
—CH₂—CH₃—O—;
and pharmaceutically acceptable salts thereof.

17. The compound according to claim 16 wherein -G²-G³-G⁴- is —CH₂—C(O)—NH—.

18. The compound of claim 1 having the Formula Ih: 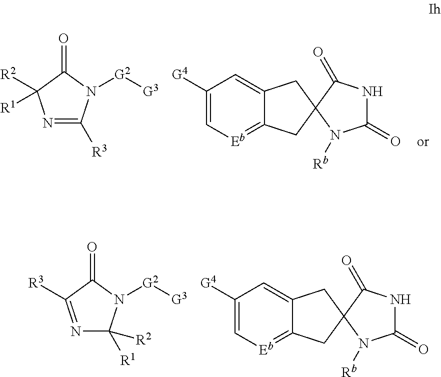
wherein:
R¹ and R² are independently selected from:
(1) —C₁₋₆alkyl, which is unsubstituted or substituted with 1-5 substituents each independently selected from:
(a) halo,
(b) —OR^{a},
(c) —C₃₋₆cycloalkyl,
(d) phenyl or heterocycle, wherein said heterocycle is selected from: pyridyl, pyrimidinyl, pyrazinyl, pyridazinyl, piperidinyl, piperazinyl, pyrrolidinyl, thienyl, morpholinyl, thiazolyl, indolyl, indazolyl, benzimidazolyl, and oxazolyl, which phenyl or heterocycle is unsubstituted or substituted with 1-5 substituents each independently selected from:
(i) halo,
(ii) —C₁₋₆alkyl, which is unsubstituted or substituted with 1-5 halo,
(iii) —OR^{a},
(iv) —NR^{b}R^{c},
(v) —CN, and
(vi) oxo;
(e) —CO₂R^{a},
(f) —C(═O)NR^{b}R^{c},
(g) —S(O)ᵥR^{d},
(h) —CN,
(i) —NR^{b}R^{c},
(j) —N(R^{b})C(═O)R^{a},
(k) —N(R^{b})SO₂R^{d},
(l) —CF₃,
(m) —O—CO₂R^{d},
(n) —O—(C═O)—NR^{b}R^{c},
(o) —NR^{b}—(C═O)—NR^{b}R^{c}, and
(p) —C(═O)R^{a}, and
(2) phenyl or heterocycle, wherein said heterocycle is selected from: pyridyl, pyrimidinyl, pyrazinyl, pyridazinyl, piperidinyl, piperazinyl, pyrrolidinyl, thienyl, morpholinyl, thiazolyl and oxazolyl, which phenyl or heterocycle is unsubstituted or substituted with 1-5 halo;
or R¹ and R² and the carbon atom or atoms to which they are attached join to fowl a ring selected from cyclopropyl, cyclobutyl, cyclopentyl, cyclohexyl, cycloheptyl, cyclooctyl, cyclononyl, cyclobutenyl, cyclopentenyl, cyclohexenyl, cycloheptenyl, cyclooctenyl, dioxolanyl, dioxanyl, aziridinyl, azetidinyl, pyrrolidinyl, piperidinyl, piperazinyl, morpholinyl, tetrahydrofuranyl, tetrahydropyranyl, tetrahydrothiapyranyl, oxetanyl, thietanyl and tetrahydrothienyl, wherein the sulfur is optionally oxidized to the sulfone or sulfoxide, which ring is unsubstituted or substituted with 1-6 substituents each independently selected from:
(a) —C₁₋₆alkyl, which is unsubstituted or substituted with 1-3 substituents each independently selected from:
(i) halo,
(ii) —OR^{a},
(iii) —C₃₋₆cycloalkyl,
(iv) —CO₂R^{a},
(v) —NR^{b}R^{c},
(vi) —S(O)ᵥR^{d},
(vii) —C(═O)NR^{b}R^{c}, and
(viii) phenyl,
(b) —C₃₋₆cycloalkyl, wherein the C₃₋₆cycloalkyl group is optionally fused to the ring, and which C₃₋₆cycloalkyl group is unsubstituted or substituted with 1-3 substituents each independently selected from:
(i) halo,
(ii) —OR^{a},
(iii) —C₃₋₆cycloalkyl,
(iv) —CO₂R^{a},
(v) —NR^{b}R^{c},
(vi) —S(O)ᵥR^{d},
(vii) —C(═O)NR^{b}R^{c}, and
(viii) phenyl,
(c) phenyl or heterocycle, wherein heterocycle is selected from: pyridyl, pyrimidinyl, pyrazinyl, pyridazinyl, piperidinyl, piperazinyl, pyrrolidinyl, thienyl, imidazolyl, furanyl, tetrahydrofuranyl, thiazolyl and oxazolyl, wherein the phenyl or heterocycle is optionally fused to the ring, and which phenyl or heterocycle is unsubstituted or substituted with 1-5 substituents each independently selected from:
(i) halo,
(ii) —C₁₋₆alkyl, which is unsubstituted or substituted with 1-5 halo,
(iii) —OR^{a},
(iv) —CO₂R^{a},
(v) —O(C═O)R^{a},
(vi) —CN,
(vii) —NR^{b}R^{c},
(viii) oxo,
(ix) —C(═O)NR^{b}R^{c},
(x) —N(R^{b})C(═O)R^{a},
(xi) —N(R^{b})CO₂R^{a},
(xii) —O(C═O)NR^{b}R^{c}, and
(xiii) —S(O)ᵥR^{d},
(d) —OR^{a},
(e) —CO₂R^{a},
(f) —C(═O)NR^{b}R^{c},
(g) —S(O)ᵥR^{d},
(h) —CN,
(i) halo,
(j) —NR^{b}R^{c},
(k)—N(R^{b})C(═O)R^{a},
(l) —N(R^{b})SO₂R^{d},
(m) —O—CO₂R^{d},
(n) —O—(C═O)—NR^{b}R^{c},
(o) —NR^{b}—(C═O)—NR^{b}R^{c},
(q) —C(═O)R^{a}, and
(r) oxo;
R³ is selected from:
(1) —C₃₋₁₀cycloalkyl or benzofused —C₃₋₁₀cycloalkyl, which is unsubstituted or substituted with 1-5 halo, and
(2) phenyl, naphthyl, tetrahydronaphthyl, indanyl, biphenyl, phenanthryl, anthryl, azepinyl, azepanyl, azetidinyl, benzimidazolyl, benzisoxazolyl, benzofuranyl, benzofurazanyl, benzopyranyl, benzothiopyranyl, benzofuryl, 1,3-benzodioxolyl, benzothiazolyl, benzothienyl, benzoxazolyl, benzopyrazolyl, benzotriazolyl, chromanyl, cinnolinyl, dibenzofuranyl, dihydrobenzofuryl, dihydrobenzothienyl, dihydrobenzothiopyranyl, dihydrobenzothiopyranyl sulfone, furyl, (uranyl, imidazolidinyl, imidazolinyl, imidazolyl, indolinyl, indolyl, isochromanyl, isoindolinyl, isoquinolinyl, isothiazolidinyl, isothiazolyl, morpholinyl, naphthyridinyl, oxazolyl, oxadiazolyl, 2-oxoazepinyl, 4-oxonaphthyridinyl, 2-oxopiperazinyl, 2-oxopiperidinyl, 2-oxopyrrolidinyl, 2-oxopyridyl, 2-oxoquinolinyl, piperidyl, piperazinyl, pyrazinyl, pyrazolidinyl, pyrazolyl, pyridazinyl, pyridinyl, pyridyl, pyrimidinyl, pyrimidyl, pyrrolidinyl, pyrrolyl, quinazolinyl, quinolinyl, quinoxalinyl, tetrahydrofuranyl, tetrahydrofuryl, tetrahydroimidazopyridinyl, tetrahydroisoquinolinyl, tetrahydroquinolinyl, tetrazolyl, thiamorpholinyl, thiamorpholinyl sulfoxide, thiamorpholinyl sulfone, thiazolyl, thiazolinyl, thienofuryl, thienothienyl, thienyl, triazolyl, isoxazolyl, tetrahydrothienyl, tetrahydropyranyl, oxetanyl, tetrahydrothiapyranyl, and thietanyl, each of which is unsubstituted or substituted with 1-5 substituents each independently selected from R⁶;
-G2-G³-G⁴- is selected from the group consisting of:
—CH₂—C(O)—NH—
—CH₂—CH₂—CH₂—,
—CH₂—CH═CH—,
—CH₂—C≡C— and
—CH₂—CH₃—O—;
and pharmaceutically acceptable salts thereof.

19. The compound according to claim 18 wherein -G²-G³-G⁴- is —CH₂—C(O)—NH—.

20. A compound selected from the following group: 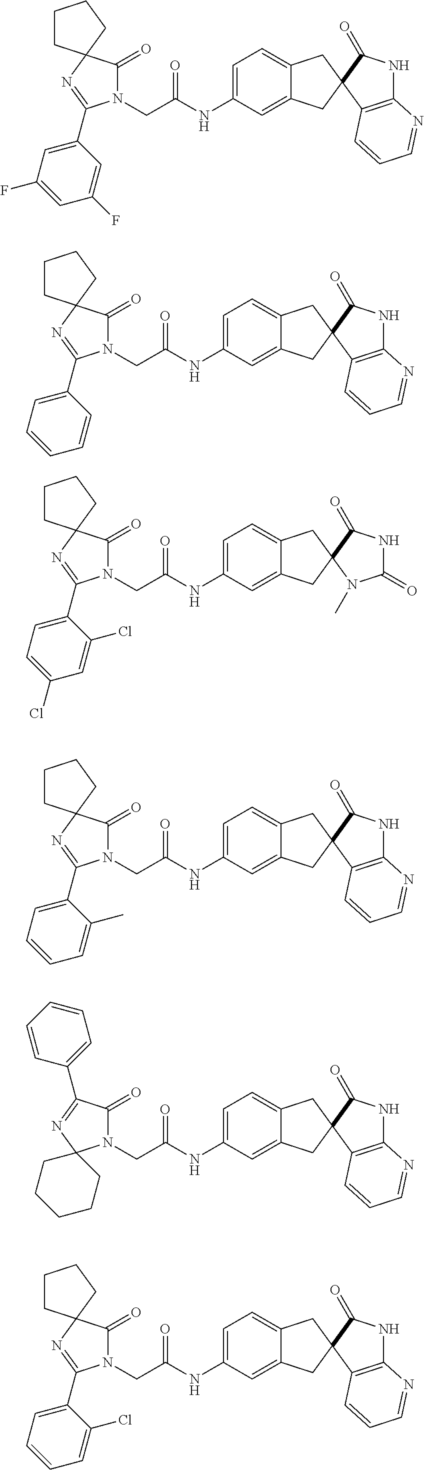 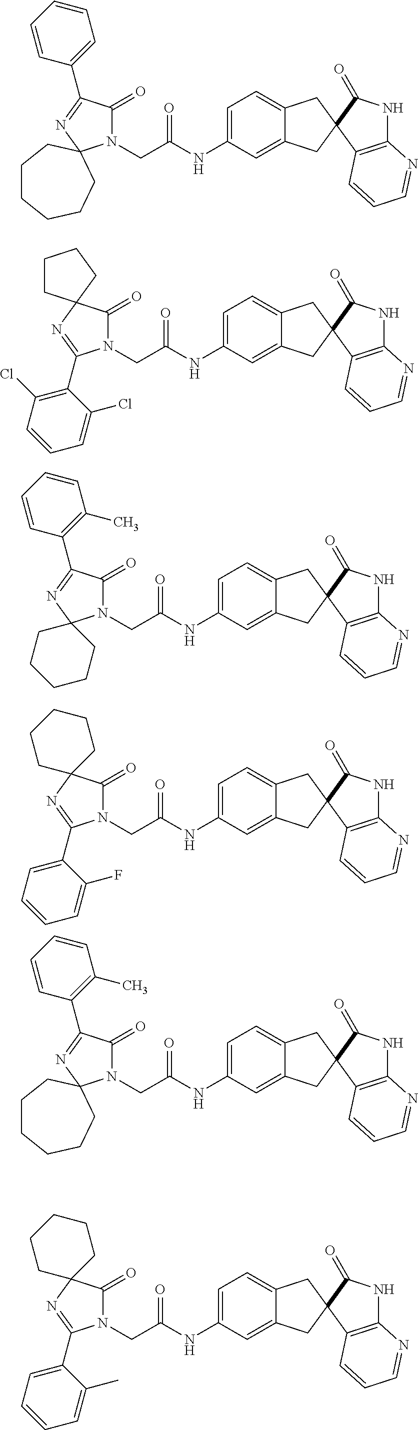 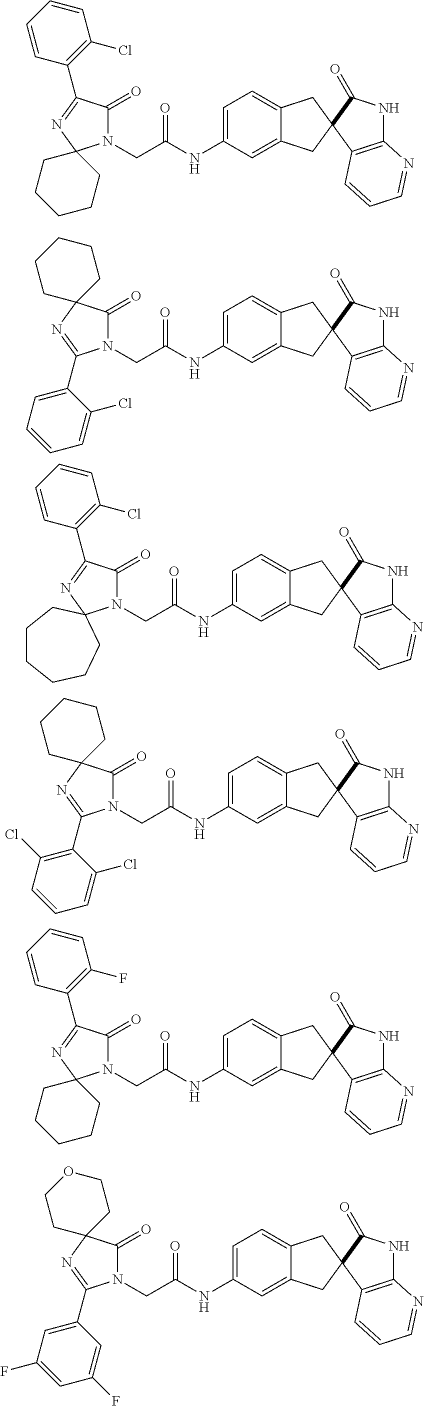 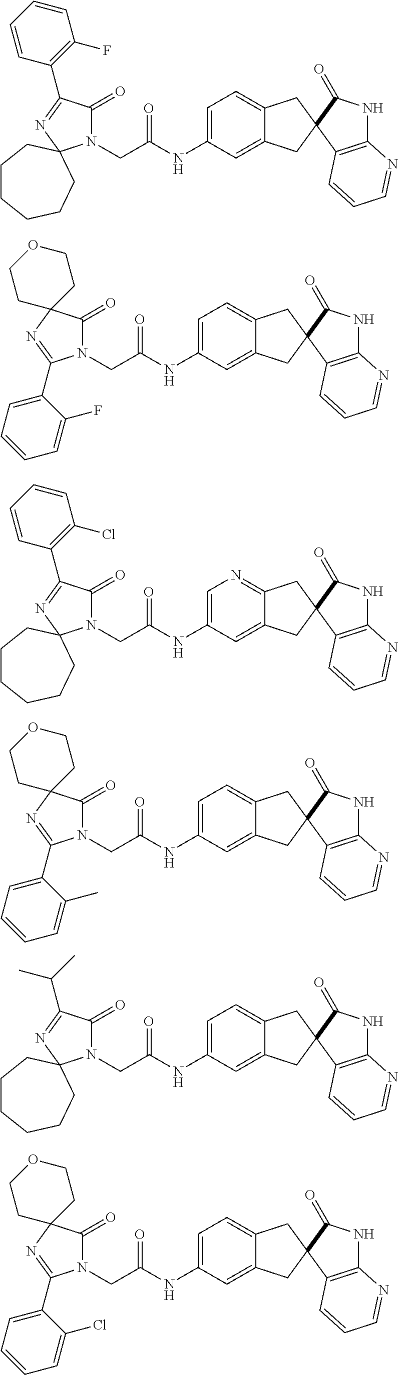 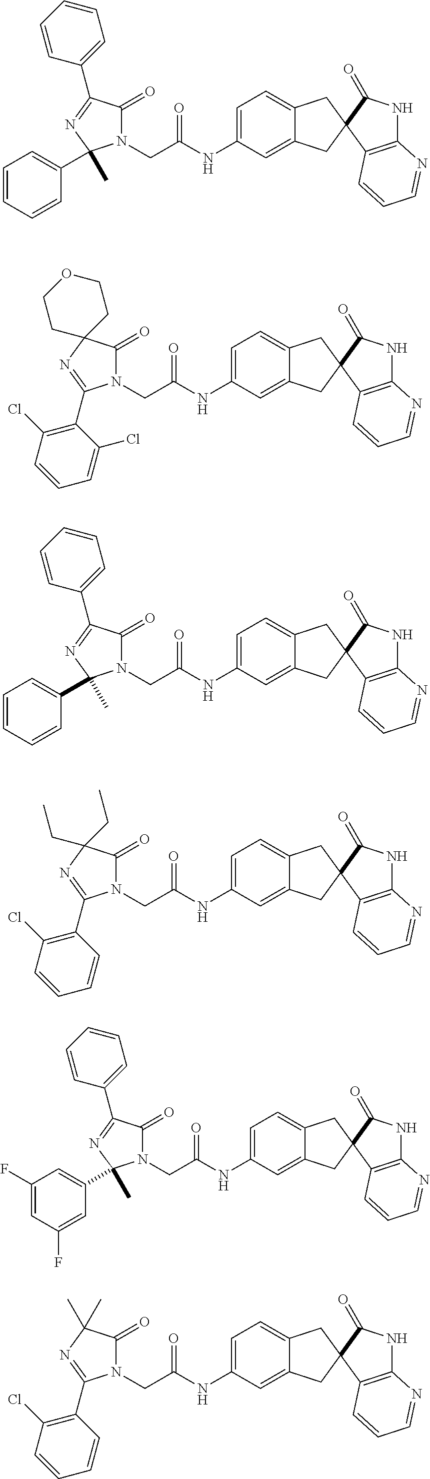 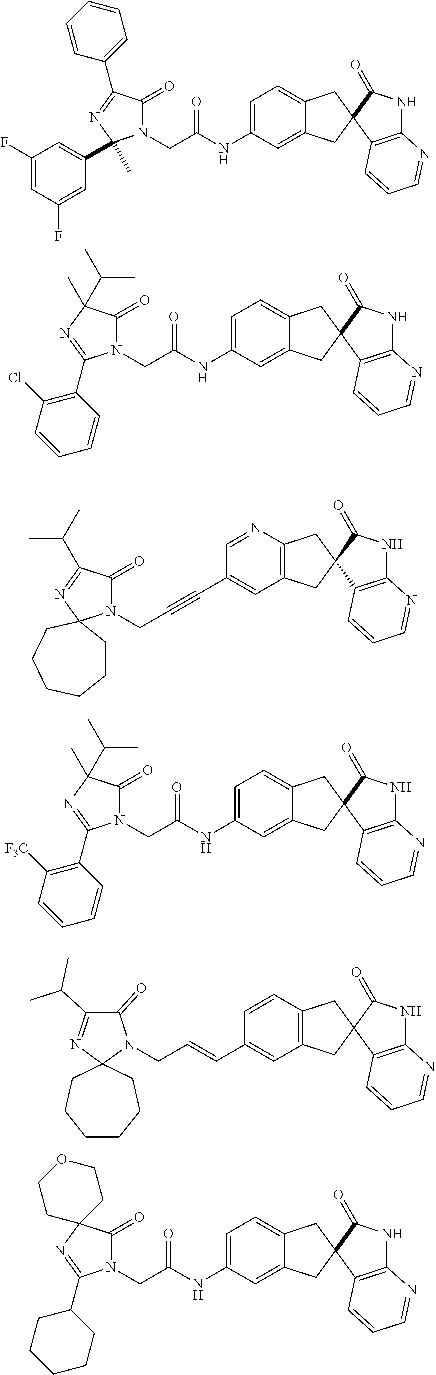 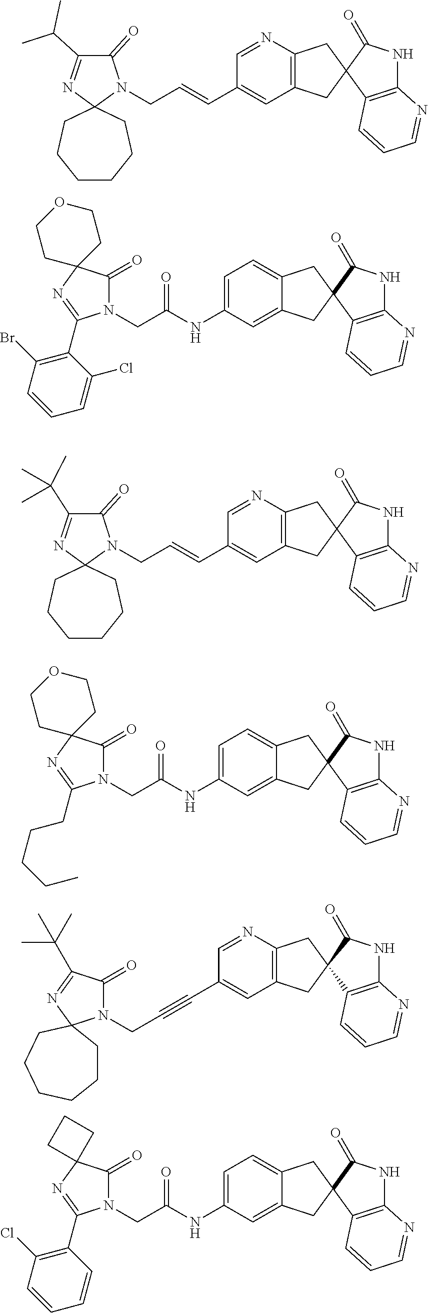 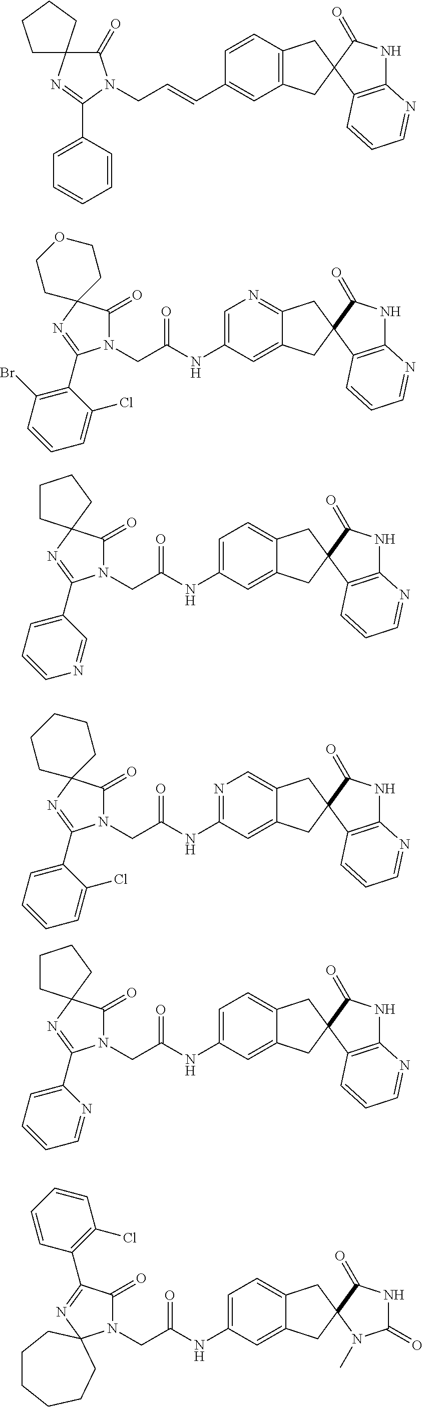 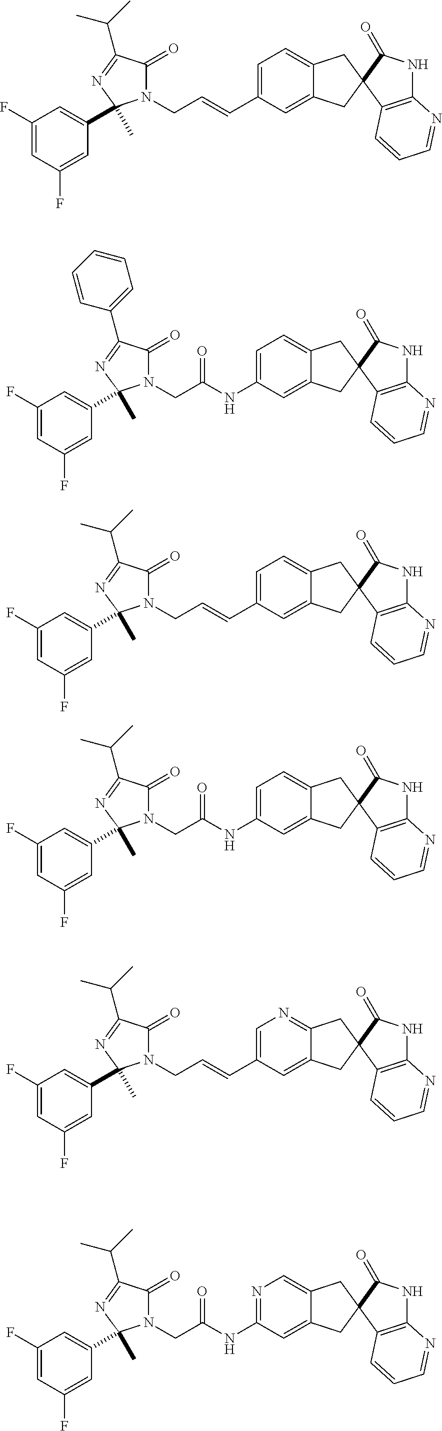 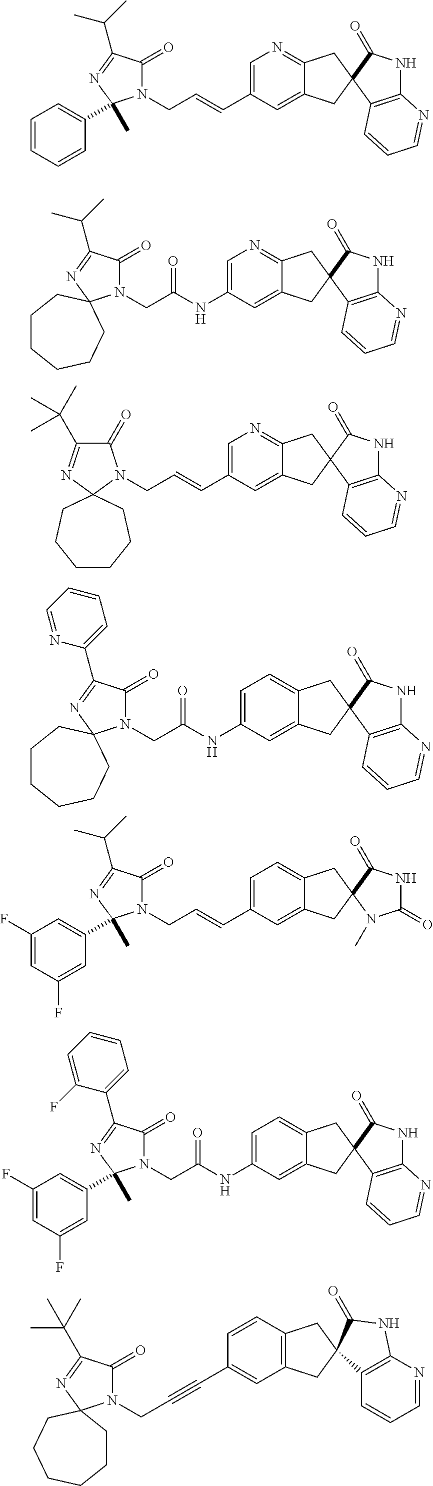 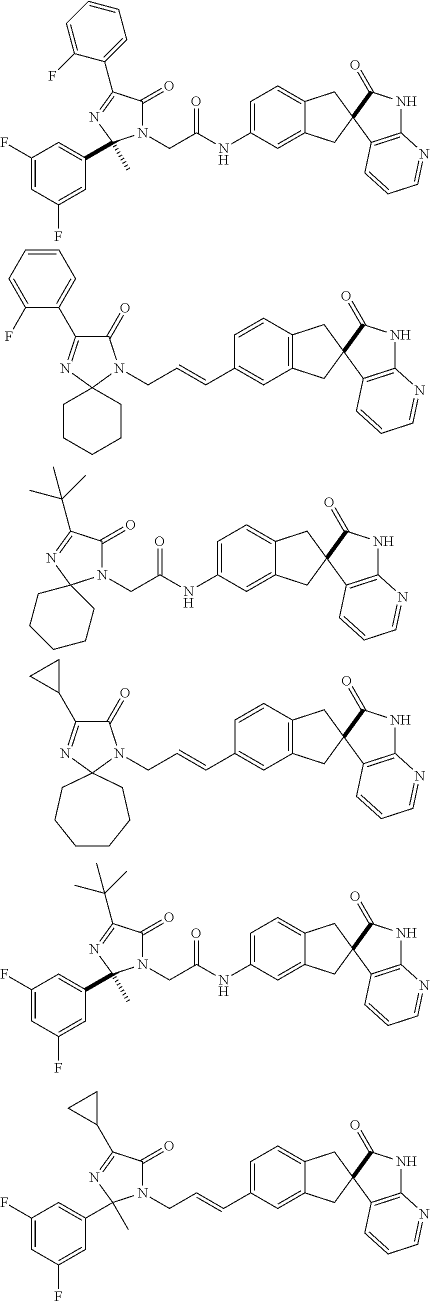 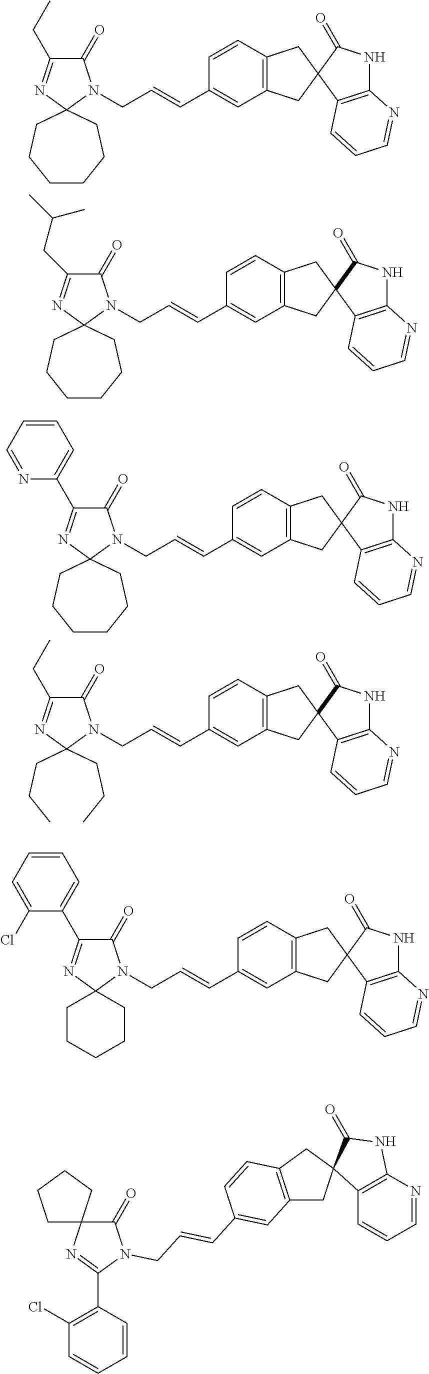 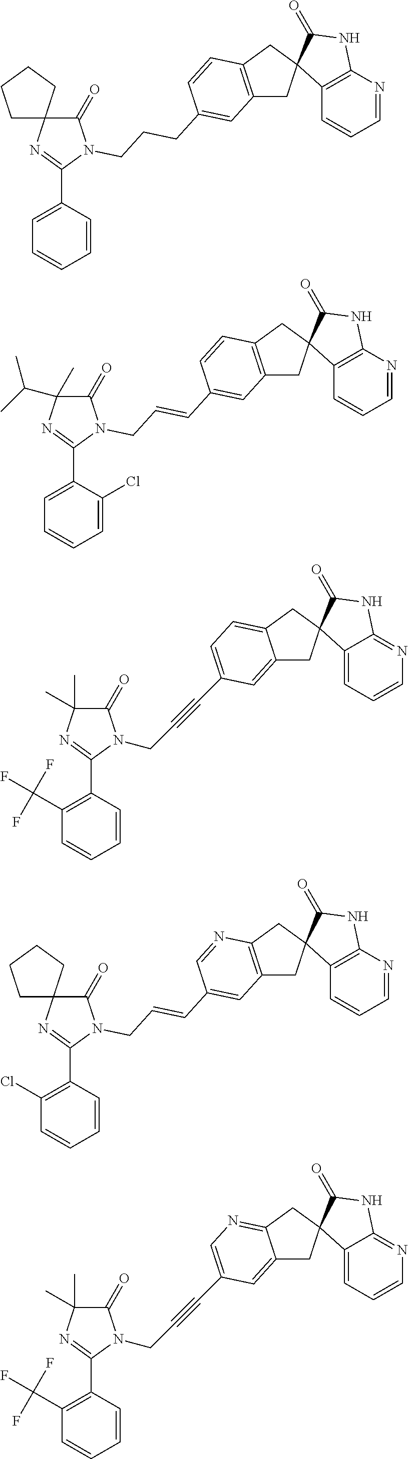 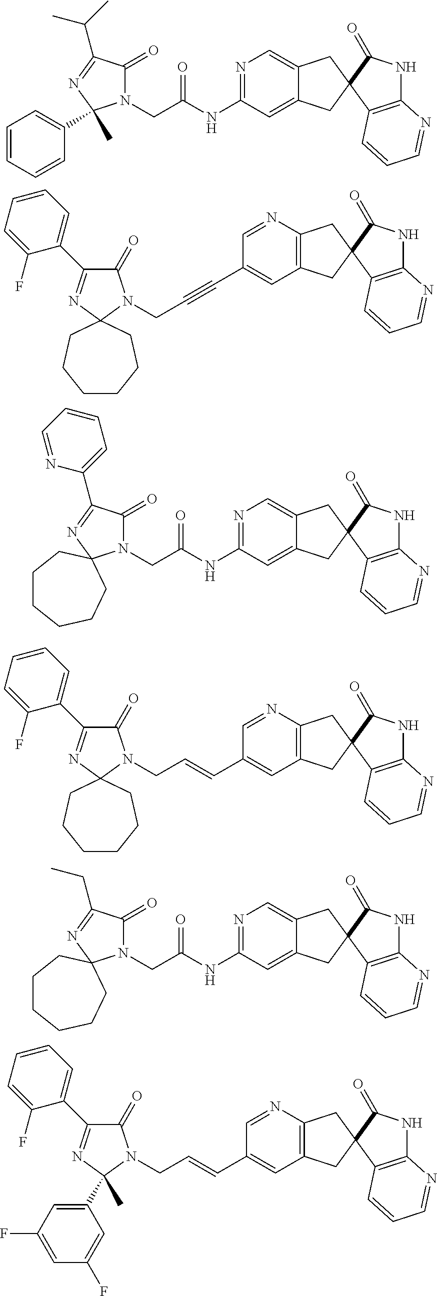 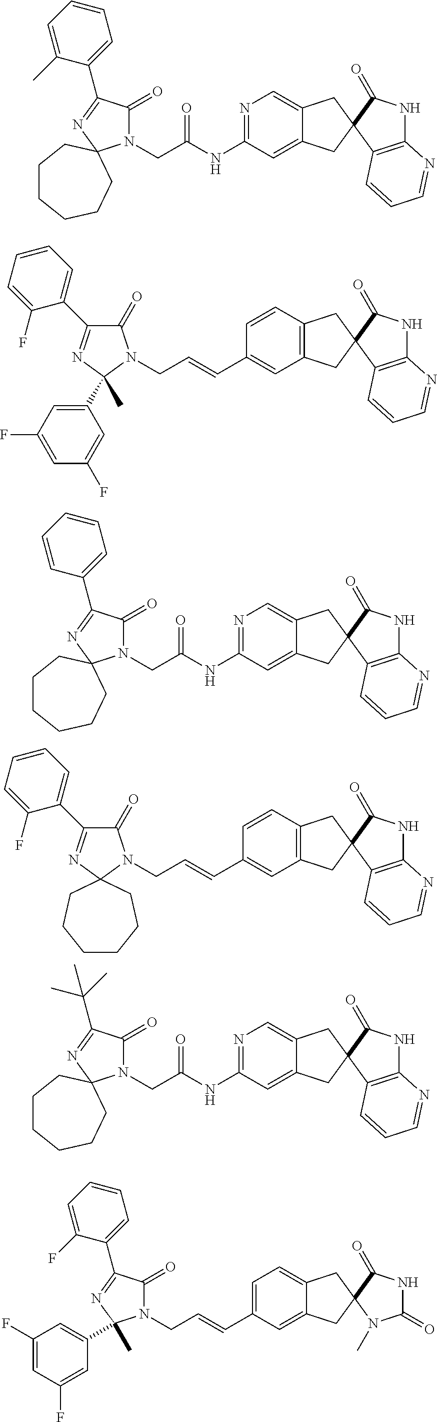 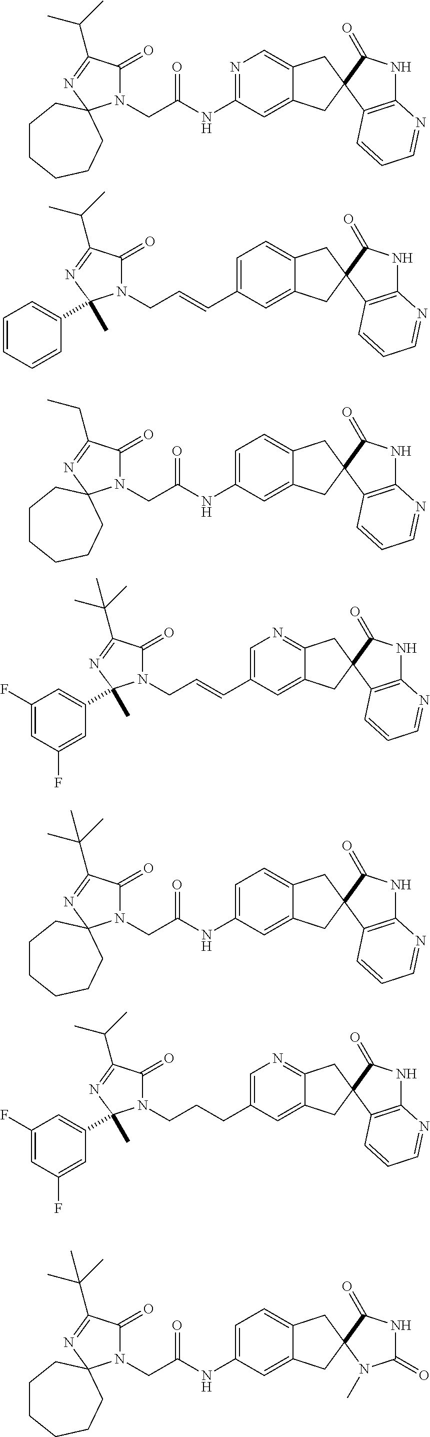 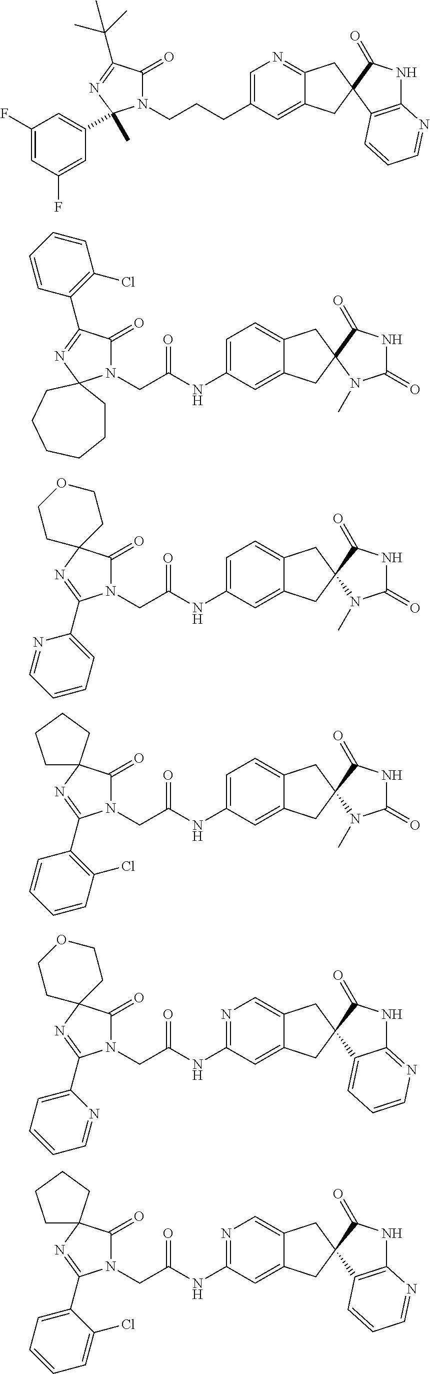 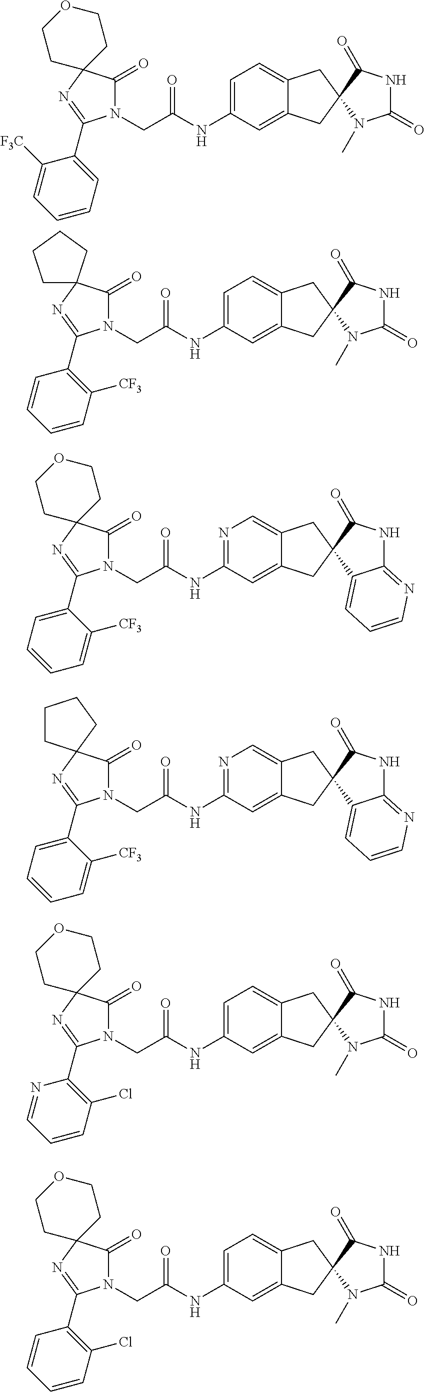 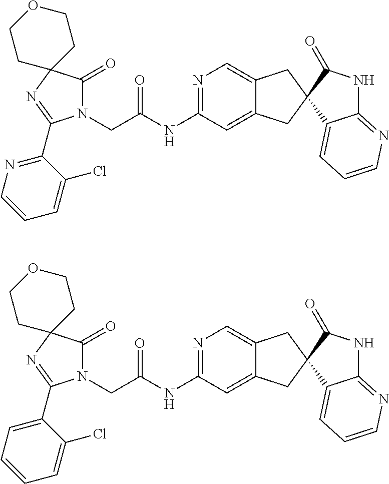
and pharmaceutically acceptable salts thereof.

21. A pharmaceutical composition which comprises an inert carrier and the compound of claim 1.

22. A method for antagonism of CGRP receptor activity in a mammal which comprises the administration of an effective amount of the compound of claim 1.

23. A method for treating, controlling, ameliorating or reducing the risk of headache, migraine or cluster headache in a mammalian patient in need of such which comprises administering to the patient a therapeutically effective amount of the compound of claim 1.

24. A method of treating or preventing migraine headaches, cluster headaches, and headaches, said method comprising the co-administration, to a person in need of such treatment, of:
a therapeutically effective amount of the compound of claim 1 or a pharmaceutically acceptable salt thereof; and
a therapeutically effective amount of a second agent selected from serotonin agonists, analgesics, anti-inflammatory agents, anti-hypertensives and anticonvulsants.
